(19) United States
(12) Patent Application Publication
(10) Pub. No.: US 2026/0226047 A1
(43) Pub. Date: Aug. 6, 2026

(54) BENZIMIDAZOLE AND AZA-BENZIMIDAZOLE BASED IL-17A MODULATORS AND USES THEREOF

(71) Applicants: DICE ALPHA, INC., Indianapolis (IN); ELI LILLY AND COMPANY, Indianapolis, IN (US)

(72) Inventors: Maureen Kay REILLY, Emerald HIlls, CA (US); Paul R. FATHEREE, San Francisco, CA (US); Michael David FREIDBERG, Belmont, CA (US); Hassane BELABED, San Francisco, CA (US); Gary Edward Lee BRANDT, Alameda, CA (US)

(21) Appl. No.: 19/152,319

(22) PCT Filed: Jan. 29, 2024
(86) PCT No.: PCT/US2024/013391

§ 371 Date: Jul. 30, 2025

Related U.S. Application Data
(60) Provisional application No. 63/482,234, filed on Jan. 30, 2023.

(51) Int. Cl. C07D 471/04; A61K 31/4184; A61K 31/4245; A61K 31/437; A61K 31/454; A61P 29/00; C07D 401/14; C07D 403/06; C07D 403/14; C07D 413/14

(52) U.S. Cl. CPC C07D 471/04; A61K 31/4184; A61K 31/4245; A61K 31/437; A61K 31/454; A61P 29/00; C07D 401/14; C07D 403/06; C07D 403/14; C07D 413/14

(57) ABSTRACT

The disclosure herein provides benzimidazole and aza-benzimidazole based compounds of Formula (I) and pharmaceutical compositions thereof, for the modulation of IL-17A, useful in the treatment of inflammatory conditions such as psoriasis.

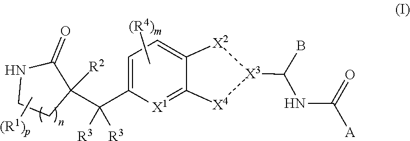

### BACKGROUND OF THE INVENTION

The IL-17 family consists of six cytokines (IL-17A through IL-17F). Interleukin-17A (IL-17A), is an established pro-inflammatory cytokine, which is involved in the induction of IL-6, IL-8, G-CSF, TNF-α, IL-1β, PGE2, and IFN-γ, as well as numerous chemokines and other effectors. IL-17A can form homodimers or heterodimers with its family member, IL-17F and can bind to both IL-17 receptors, IL-17 RA and IL-17 RC, in order to mediate signaling. IL-17A is a major pathological cytokine expressed by Th17 cells, which are involved in the pathology of inflammation and autoimmunity, and also CD8+ T cells, γδ cells, NK cells, NKT cells, macrophages and dendritic cells. Additionally, IL-17A and Th17 are necessary for defense against various microbes despite their involvement in inflammation and autoimmune disorders. Further, IL-17A can act in cooperation with other inflammatory cytokines such as TNF-α, IFN-γ, and IL-1β to mediate pro-inflammatory effects.

To date, there are a few biologics (Secukinumab and Ixekizumab) that have been approved to modulate IL-17A for the treatment of inflammatory diseases, such as psoriasis, ankylosing spondylitis, and psoriatic arthritis. These treatments require injection to a patient as they are not readily absorbed by the gut when orally ingested. Further, these approved biologic treatments have a high cost of entry for patients, limiting the availability to the patient population in need thereof.

There are a few small molecule modulators of IL-17A that have been approved for oral administration. However, while these have the convenience of oral administration and a lower cost of entry for patients, they lack the efficacy of approved biologics. Therefore, there exists a need for the development of potent small molecule IL-17A modulators for the treatment of inflammatory diseases and other associated disorders.

### SUMMARY OF THE INVENTION

In certain aspects the present disclosure provides a compound represented by the structure of Formula (I):

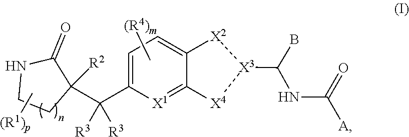

or a pharmaceutically acceptable salt thereof wherein:
A is selected from 5- to 6-membered heteroaryl and C₃₋₆ carbocycle, any of which is optionally substituted with one or more substituents independently selected from:
   halogen, —OR¹¹, —SR¹¹, —N(R¹¹)₂, —C(O)R¹¹, —C(O)N(R¹¹)₂, —N(R¹¹)C(O)R¹¹, —N(R¹¹)S(O)₂R¹¹, —C(O)OR¹¹, —OC(O)R¹¹, —S(O)R¹¹, —S(O)₂R¹¹, —NO₂, —CN; and
   C₁₋₁₀ alkyl optionally substituted with one or more substituents independently selected from halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —S(O)R¹¹, —S(O)₂R¹¹, —NO₂, ═O, ═S, ═N(R¹¹), —CN, C₃₋₁₀ carbocycle and 3- to 10-membered heterocycle; wherein the C₃₋₁₀ carbocycle and 3- to 10-membered heterocycle are each optionally substituted with one or more substituents selected from: halogen, —OR¹¹, —N(R¹¹)₂, —C(O)R¹¹, —C(O)N(R¹¹)₂, —N(R¹¹)C(O)R¹¹, —C(O)OR¹¹, —OC(O)R¹¹, —NO₂, ═O, ═N(R¹¹), and —CN;
B is selected from —C(H)(R⁵)₂ and C₃₋₁₀ carbocycle, wherein each C₃₋₁₀ carbocycle is optionally substituted with one or more substituents independently selected from:
   halogen, —OR¹², —SR¹², —N(R¹²)₂, —C(O)R¹², —C(O)N(R¹²)₂, —N(R¹²)C(O)R¹², —N(R¹²)S(O)₂R¹², —C(O)OR¹², —OC(O)R¹², —S(O)R¹², —S(O)₂R¹², —NO₂, ═O, ═S, ═N(R¹²), —CN;
   C₁₋₁₀ alkyl and C₃₋₁₀ carbocycle, any of which is optionally substituted with one or more substituents independently selected from halogen, —OR¹², —SR¹², —N(R¹²)₂, —C(O)R¹², —C(O)N(R¹²)₂, —N(R¹²)C(O)R¹², —C(O)OR¹², —OC(O)R¹², —S(O)R¹², —S(O)₂R¹², —NO₂, ═O, ═S, ═N(R¹²), —CN, C₃₋₁₀ carbocycle and 3- to 10-membered heterocycle; wherein the C₃₋₁₀ carbocycle and 3- to 10-membered heterocycle are each optionally substituted with one or more substituents selected from: halogen, —OR¹², —N(R¹²)₂, —C(O)R¹², —C(O)N(R¹²)₂, —N(R¹²)C(O)R¹². —C(O)OR¹², —OC(O)R¹², —NO₂, ═O, ═N(R¹²), and —CN;
each R⁵ is independently selected at each occurrence from (i), (ii), and (iii):
   (i) halogen, —OR¹³, —SR¹³, —N(R¹³)₂, —C(O)R¹³, —C(O)N(R¹³)₂, —N(R¹³) C(O)R¹³, —N(R¹³)S(O)₂R¹³, —C(O)OR¹³, —OC(O)R¹³, —S(O)R¹³, —S(O)₂R¹³, —NO₂, ═O, ═S, ═N(R¹³), —CN;
   (ii) C₁₋₁₀ alkyl optionally substituted with one or more substituents independently selected from halogen, —OR¹³, —SR¹³, —N(R¹³)₂, —C(O)R¹³, —C(O)N(R¹³)₂, —N(R¹³) C(O)R¹³, —N(R¹³)S(O)₂R¹³, —C(O)OR¹³, —OC(O)R¹³, —S(O)R¹³, —S(O)₂R¹³, —NO₂, ═O, ═S, ═N(R¹³), —CN; and
   (iii) C₃₋₁₀ carbocycle optionally substituted with one or more substituents independently selected from:
      halogen, —OR¹³, —SR¹³, —N(R¹³)₂, —C(O)R¹³, —C(O)N(R¹³)₂, —N(R¹³) C(O)R¹³, —N(R¹³)S(O)₂R¹³, —C(O)OR¹³, —OC(O)R¹³, —S(O)R¹³, —S(O)₂R¹³, —NO₂, ═O, ═S, ═N(R¹³), —CN; and
      C₁₋₁₀ alkyl optionally substituted with one or more substituents independently selected from halogen, —OR¹³, —SR¹³, —N(R¹³)₂, —C(O)R¹³, —C(O)N(R¹³)₂, —N(R¹³) C(O)R¹³, —C(O)OR¹³, —OC(O)R¹³, —S(O)R¹³, —S(O)₂R¹³, —NO₂, ═O, ═S, ═N(R¹³), and —CN;
each R¹ is independently selected at each occurrence from halogen, —OR¹⁴, —SR¹⁴, —N(R¹⁴)₂, —C(O)R¹⁴, —C(O)N(R¹⁴)₂, —N(R¹⁴)C(O)R¹⁴, —N(R¹⁴)S(O)₂R¹⁴, —C(O)OR¹⁴, —OC(O)R¹⁴, —S(O)R¹⁴, —S(O)₂R¹⁴, —NO₂, ═O, ═S, ═N(R¹⁴), —CN; and C₁₋₁₀ alkyl optionally substituted with one or more substituents independently selected from halogen, —OR¹⁴, —SR¹⁴, —N(R¹⁴)₂, —C(O)R¹⁴, —C(O)N(R¹⁴)₂, —N(R¹⁴)C(O)R¹⁴, —C(O)OR¹⁴, —OC(O)R¹⁴, —S(O)R¹⁴, —S(O)₂R¹⁴, —NO₂, ═O, ═S, ═N(R¹⁴), and —CN; or
   one R¹ on each of two adjacent carbons come together to form an optionally substituted C₃₋₆ carbocycle together with carbons atoms to which they are attached, wherein the C₃₋₆ carbocycle is optionally substituted with one or more substituents independently selected from halogen, —OR¹⁹, —SR¹⁹, —N(R¹⁹)₂, —C(O)R¹⁹, —C(O)N(R¹⁹)₂, —N(R¹⁹)C(O)R¹⁹, —N(R¹⁹)S(O)₂R¹⁹, —C(O)OR¹⁹, —OC(O)R¹⁹, —S(O)R¹⁹, —S(O)₂R¹⁹, —NO₂, ═O, —S, ═N(R¹⁹), —CN, C₁₋₆ alkyl, and C₁₋₆ haloalkyl;
R² is selected from hydrogen, halogen, —OR¹⁵, —SR¹⁵, —N(R¹⁵)₂, —C(O)R¹⁵, —C(O)N(R¹⁵)₂, —N(R¹⁵)C(O)R¹⁵, —N(R¹⁵) S(O)₂R¹⁵, —C(O)OR¹⁵, —OC(O)R¹⁵, —S(O)R¹⁵, —S(O)₂R¹⁵, —NO₂, —CN; and C₁₋₆ alkyl optionally substituted with one or more substituents independently selected from halogen, —OR¹⁵, —SR¹⁵, —N(R¹⁵)₂, —C(O)R¹⁵, —C(O)N(R¹⁵)₂, —N(R¹⁵)C(O)R¹⁵, —C(O)OR¹⁵, —OC(O)R¹⁵, —S(O)R¹⁵, —S(O)₂R¹⁵, —NO₂, ═O, ═S, ═N(R¹⁵), and —CN;
each R³ is independently selected at each occurrence from hydrogen, halogen, —OR¹⁶, —SR¹⁶, —N(R¹⁶)₂, —C(O)R¹⁶, —C(O)N(R¹⁶)₂, —N(R¹⁶)C(O)R¹⁶, —N(R¹⁶)S(O)₂R¹⁶, —C(O)OR¹⁶, —OC(O)R¹⁶, —S(O)R¹⁶, —S(O)₂R¹⁶, —NO₂, —CN; and C₁₋₆ alkyl optionally substituted with one or more substituents independently selected from halogen, —OR¹⁶, —SR¹⁶, —N(R¹⁶)₂, —C(O)R¹⁶, —C(O)N(R¹⁶)₂, —N(R¹⁶)C(O)R¹⁶, —C(O)OR¹⁶, —OC(O)R¹⁶, —S(O)R¹⁶, —S(O)₂R¹⁶, —NO₂, ═O, ═S, ═N(R¹⁶), and —CN; or
R² and one R³ may come together to form C₃₋₆ carbocycle optionally substituted with one or more substituents independently selected from halogen, —OR²⁰, —SR²⁰, —N(R²⁰)₂, —C(O)R²⁰, —C(O)N(R²⁰)₂, —N(R²⁰)C(O)R²⁰, —N(R²⁰)S(O)₂R²⁰, —C(O)OR²⁰, —OC(O)R²⁰, —S(O)R²⁰, —S(O)₂R²⁰, —NO₂, ═O, ═S, ═N(R¹⁹), —CN, C₁₋₆ alkyl, and C₁₋₆ haloalkyl;
each R⁴ is independently selected at each occurrence from halogen, —OR¹⁷, —SR¹⁷, —N(R¹⁷)₂, —C(O)R¹⁷, —C(O)N(R¹⁷)₂, —N(R¹⁷)C(O)R¹⁷, —N(R¹⁷)S(O)₂R¹⁷, —C(O)OR¹⁷, —OC(O)R¹⁷, —S(O)R¹⁷, —S(O)₂R¹⁷, —NO₂, —CN; and C₁₋₁₀ alkyl optionally substituted with one or more substituents independently selected from halogen, —OR¹⁷, —SR¹⁷, —N(R¹⁷)₂, —C(O)R¹⁷, —C(O)N(R¹⁷)₂, —N(R¹⁷)C(O)R¹⁷, —C(O)OR¹⁷, —OC(O)R¹⁷, —S(O)R¹⁷, —S(O)₂R¹⁷, —NO₂, ═O, ═S, ═N(R¹⁷), and —CN;
X¹ is independently selected from N and C(R¹⁰);
X³ is C;
each X² and X⁴ is independently selected from N, N(H) and C(R¹⁰), wherein
   when X² is N(H), X⁴ is selected from N and C(R¹⁰); the bond between X² and X³ is a single bond and the bond between X³ and X⁴ is a double bond; or when X⁴ is N(H), X² is selected from N and C(R¹⁰); the bond between X² and X³ is a double bond and the bond between X³ and X⁴ is a single bond;
   wherein at least one of X² and X⁴ is N(H);
R¹⁰ is selected from hydrogen, halogen, —OR¹⁸, —SR¹⁸, —N(R¹⁸)₂, —C(O)R¹⁸, —C(O)OR¹⁸, —NO₂, —CN, C₁₋₆ alkyl, C₁₋₆ haloalkyl, —O—C₁₋₆ haloalkyl, and —O—C₁₋₆ alkyl;
R¹¹, R¹², R¹³, R¹⁴, R¹⁵, R¹⁶, R¹⁷, R¹⁸, R¹⁹, and R²⁰ are each independently selected at each occurrence from:
   hydrogen;
   C₁₋₆ alkyl optionally substituted with one more substituents independently selected from halogen, —O—C₁₋₆ alkyl, —O—C₁₋₆ haloalkyl, —NH₂, —NO₂, ═O, —CN, C₃₋₁₀ carbocycle and 3- to 10-membered heterocycle, wherein each C₃₋₁₀ carbocycle and 3- to 10-membered heterocycle are optionally substituted with one or more substituents independently selected from: halogen, —OH, —O—C₁₋₆ alkyl, —O—C₁₋₆ haloalkyl, —NH₂, —NO₂, ═O, and —CN; and
   C₃₋₁₀ carbocycle and 3- to 10-membered heterocycle optionally substituted with one or more substituents independently selected from: halogen, —OH, —O—C₁₋₆ alkyl, —O—C₁₋₆ haloalkyl, —NH₂, —NO₂, ═O, and —CN;
n is selected from 1 and 2;
m is selected from 0, 1 and 2; and
p is selected from 0, 1, 2, 3, 4, 5, and 6.

In certain aspects, the disclosure provides a pharmaceutical composition comprising a pharmaceutically acceptable excipient and a compound or salt of Formula (I), (I-a), (I-b), (I-c), (I-d), (I-e), or (I-f).

In certain aspects, the disclosure provides a method of modulating IL-17A in a subject in need thereof, comprising administering to the subject a compound or salt of Formula (I), (I-a), (I-b), (I-c), (I-d), (I-e), or (I-f), or a pharmaceutical composition thereof.

In certain aspects, the disclosures provides a method of treating an inflammatory disease or condition comprising administering to the subject a compound or salt of Formula (I), (I-a), (I-b), (I-c), (I-d), (I-e), or (I-f), or a pharmaceutical composition thereof. In some embodiments, the inflammatory disease or condition is selected from plaque psoriasis, guttate psoriasis, inverse psoriasis, pustular psoriasis, erythrodermic psoriasis, psoriatic arthritis, ankylosing spondylitis, hidradenitis suppurativa, rheumatoid arthritis, palmoplantar psoriasis, spondyloarthritis, and Non-infectious Uveitis.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### DETAILED DESCRIPTION OF THE INVENTION

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### Definitions

Unless defined otherwise, all technical and scientific terms used herein have the same meaning as is commonly understood by one of skill in the art to which this invention belongs. All patents and publications referred to herein are incorporated by reference.

As used in the specification and claims, the singular form “a”, “an” and “the” includes plural references unless the context clearly dictates otherwise.

“Alkyl” refers to a straight or branched hydrocarbon chain monovalent radical consisting solely of carbon and hydrogen atoms, containing no unsaturation, and preferably having from one to twelve carbon atoms (i.e., C₁-C₁₂ alkyl). The alkyl is attached to the remainder of the molecule through a single bond. In certain embodiments, an alkyl comprises one to twelve carbon atoms (i.e., C₁-C₁₂ alkyl). In certain embodiments, an alkyl comprises one to eight carbon atoms (i.e., C₁-C₈ alkyl). In other embodiments, an alkyl comprises one to five carbon atoms (i.e., C₁-C₈alkyl). In other embodiments, an alkyl comprises one to four carbon atoms (i.e., C₁-C₄ alkyl). In other embodiments, an alkyl comprises one to three carbon atoms (i.e., C₁-C₃ alkyl). In other embodiments, an alkyl comprises one to two carbon atoms (i.e., C₁-C₂ alkyl). In other embodiments, an alkyl comprises one carbon atom (i.e., C₁ alkyl). In other embodiments, an alkyl comprises five to fifteen carbon atoms (i.e., C₅-C₁₅ alkyl). In other embodiments, an alkyl comprises five to eight carbon atoms (i.e., C₅-C₈ alkyl). In other embodiments, an alkyl comprises two to five carbon atoms (i.e., C₂-C₅ alkyl). In other embodiments, an alkyl comprises three to five carbon atoms (i.e., C₃-C₅ alkyl). For example, the alkyl group may be attached to the rest of the molecule by a single bind, such as, methyl, ethyl, 1-propyl (n-propyl), 1-methylethyl (iso-propyl), 1-butyl (n-butyl), 1-methylpropyl (sec-butyl), 2-methylpropyl (iso-butyl), 1,1-dimethylethyl (tert-butyl), 1-pentyl (n-pentyl), and the like.

“Alkenyl” refers to a straight or branched hydrocarbon chain radical group consisting solely of carbon and hydrogen atoms, containing at least one carbon-carbon double bond, and preferably having from two to twelve carbon atoms (i.e., C₂-C₁₂ alkenyl). In certain embodiments, an alkenyl comprises two to eight carbon atoms (i.e., C₂-C₈ alkenyl). In certain embodiments, an alkenyl comprises two to six carbon atoms (i.e., C₂-C₆ alkenyl). In other embodiments, an alkenyl comprises two to four carbon atoms (i.e., C₂-C₄ alkenyl). The alkenyl is attached to the rest of the molecule by a single bond, for example, ethenyl (i.e., vinyl), prop-1-enyl (i.e., allyl), but-1-enyl, pent-1-enyl, penta-1,4-dienyl, and the like.

“Alkynyl” refers to a straight or branched hydrocarbon chain radical group consisting solely of carbon and hydrogen atoms, containing at least one carbon-carbon triple bond, and preferably having from two to twelve carbon atoms (i.e., C₂-C₁₂ alkynyl). In certain embodiments, an alkynyl comprises two to eight carbon atoms (i.e., C₂-C₈ alkynyl). In other embodiments, an alkynyl comprises two to six carbon atoms (i.e., C₂-C₆ alkynyl). In other embodiments, an alkynyl comprises two to four carbon atoms (i.e., C₂-C₄ alkynyl). The alkynyl is attached to the rest of the molecule by a single bond, for example, ethynyl, propynyl, butynyl, pentynyl, hexynyl, and the like.

“Alkylene” refers to a straight divalent hydrocarbon chain linking the rest of the molecule to a radical group, consisting solely of carbon and hydrogen, containing no unsaturation, and preferably having from one to twelve carbon atoms, for example, methylene, ethylene, propylene, butylene, and the like. The alkylene chain is attached to the rest of the molecule through a single bond and to the radical group through a single bond. The points of attachment of the alkylene chain to the rest of the molecule and to the radical group are through the terminal carbons respectively. Alkylene chain may be optionally substituted by one or more substituents such as those substituents described herein. In certain embodiments, an alkylene comprises one to ten carbon atoms (i.e., C₁-C₁₀ alkylene). In certain embodiments, an alkylene comprises one to eight carbon atoms (i.e., C₁-C₈ alkylene). In other embodiments, an alkylene comprises one to five carbon atoms (i.e., C₁-C₅ alkylene). In other embodiments, an alkylene comprises one to four carbon atoms (i.e., C₁-C₄ alkylene). In other embodiments, an alkylene comprises one to three carbon atoms (i.e., C₁-C₃ alkylene). In other embodiments, an alkylene comprises one to two carbon atoms (i.e., C₁-C₂ alkylene). In other embodiments, an alkylene comprises one carbon atom (i.e., C₁ alkylene). In other embodiments, an alkylene comprises five to eight carbon atoms (i.e., C₅-C₈ alkylene). In other embodiments, an alkylene comprises two to five carbon atoms (i.e., C₂-C₅ alkylene). In other embodiments, an alkylene comprises three to five carbon atoms (i.e., C₃-C₅ alkylene).

“Alkenylene” refers to a straight divalent hydrocarbon chain linking the rest of the molecule to a radical group, consisting solely of carbon and hydrogen, containing at least one carbon-carbon double bond, and preferably having from two to twelve carbon atoms. The alkenylene chain is attached to the rest of the molecule through a single bond and to the radical group through a single bond. The points of attachment of the alkenylene chain to the rest of the molecule and to the radical group are through the terminal carbons respectively. Alkenylene chain may be optionally substituted by one or more substituents such as those substituents described herein. In certain embodiments, an alkenylene comprises two to ten carbon atoms (i.e., C₂-C₁₀ alkenylene). In certain embodiments, an alkenylene comprises two to eight carbon atoms (i.e., C₂-C₈ alkenylene). In other embodiments, an alkenylene comprises two to five carbon atoms (i.e., C₂-C₅ alkenylene). In other embodiments, an alkenylene comprises two to four carbon atoms (i.e., C₂-C₄ alkenylene). In other embodiments, an alkenylene comprises two to three carbon atoms (i.e., C₂-C₃ alkenylene). In other embodiments, an alkenylene comprises two carbon atom (i.e., C₂ alkenylene). In other embodiments, an alkenylene comprises five to eight carbon atoms (i.e., C₅-C₈ alkenylene). In other embodiments, an alkenylene comprises three to five carbon atoms (i.e., C₃-C₅ alkenylene).

“Alkynylene” refers to a straight divalent hydrocarbon chain linking the rest of the molecule to a radical group, consisting solely of carbon and hydrogen, containing at least one carbon-carbon triple bond, and preferably having from two to twelve carbon atoms. The alkynylene chain is attached to the rest of the molecule through a single bond and to the radical group through a single bond. The points of attachment of the alkynylene chain to the rest of the molecule and to the radical group are through the terminal carbons respectively. Alkynylene chain may be optionally substituted by one or more substituents such as those substituents described herein. In certain embodiments, an alkynylene comprises two to ten carbon atoms (i.e., C₂-C₁₀ alkynylene). In certain embodiments, an alkynylene comprises two to eight carbon atoms (i.e., C₂-C₈ alkynylene). In other embodiments, an alkynylene comprises two to five carbon atoms (i.e., C₂-C₅ alkynylene). In other embodiments, an alkynylene comprises two to four carbon atoms (i.e., C₂-C₄ alkynylene). In other embodiments, an alkynylene comprises two to three carbon atoms (i.e., C₂-C₃ alkynylene). In other embodiments, an alkynylene comprises two carbon atom (i.e., C₂ alkynylene). In other embodiments, an alkynylene comprises five to eight carbon atoms (i.e., C₅-C₈ alkynylene). In other embodiments, an alkynylene comprises three to five carbon atoms (i.e., C₃-C₅ alkynylene).

The term “C_{x-y}” when used in conjunction with a chemical moiety, such as alkyl, alkenyl, or alkynyl is meant to include groups that contain from x to y carbons in the chain. For example, the term “C₁₋₆ alkyl” refers to substituted or unsubstituted saturated hydrocarbon groups, including straight-chain alkyl and branched-chain alkyl groups that contain from 1 to 6 carbons. The term —C_{x-y} alkylene-refers to a substituted or unsubstituted alkylene chain with from x to y carbons in the alkylene chain. For example, —C₁₋₆ alkylene- may be selected from methylene, ethylene, propylene, butylene, pentylene, and hexylene, any one of which is optionally substituted.

The terms “C_{x-y} alkenyl” and “C_{x-y} alkynyl” refer to unsaturated aliphatic groups analogous in length and possible substitution to the alkyls described above, but that contain at least one double or triple bond, respectively. The term —C_{x-y} alkenylene-refers to a substituted or unsubstituted alkenylene chain with from x to y carbons in the alkenylene chain. For example, —C₂₋₆ alkenylene- may be selected from ethenylene, propenylene, butenylene, pentenylene, and hexenylene, any one of which is optionally substituted. An alkenylene chain may have one double bond or more than one double bond in the alkenylene chain. The term —C_{x-y}alkynylene- refers to a substituted or unsubstituted alkynylene chain with from x to y carbons in the alkynylene chain. For example, —C₂₋₆ alkynylene- may be selected from ethynylene, propynylene, butynylene, pentynylene, and hexynylene, any one of which is optionally substituted. An alkynylene chain may have one triple bond or more than one triple bond in the alkynylene chain.

The term “carbocycle” as used herein refers to a saturated, unsaturated or aromatic ring in which each atom of the ring is carbon. Carbocycle include 3- to 10-membered monocyclic rings and 6- to 12-membered bicyclic rings. Each ring of a bicyclic carbocycle may be selected from saturated, unsaturated, and aromatic rings. Bicyclic carbocycles may be fused, bridged or spiro-ring systems. In some embodiments, the carbocycle is an aryl. In some embodiments, the carbocycle is a cycloalkyl. In some embodiments, the carbocycle is a cycloalkenyl. In an exemplary embodiment, an aromatic ring, e.g., phenyl, may be fused to a saturated or unsaturated ring, e.g., cyclohexane, cyclopentane, or cyclohexene. Any combination of saturated, unsaturated and aromatic bicyclic rings, as valence permits, are included in the definition of carbocyclic. Exemplary carbocycles include cyclopentyl, cyclohexyl, cyclohexenyl, adamantyl, phenyl, indanyl, and naphthyl. Carbocycle may be optionally substituted by one or more substituents such as those substituents described herein.

“Cycloalkyl” refers to a stable fully saturated monocyclic or polycyclic hydrocarbon radical consisting solely of carbon and hydrogen atoms, which includes fused or bridged ring systems, and preferably having from three to twelve carbon atoms (i.e., C₃₋₁₂ cycloalkyl). In certain embodiments, a cycloalkyl comprises three to ten carbon atoms (i.e., C₃₋₁₀ cycloalkyl). In other embodiments, a cycloalkyl comprises five to seven carbon atoms (i.e., C₅₋₇ cycloalkyl). The cycloalkyl may be attached to the rest of the molecule by a single bond. Examples of monocyclic cycloalkyls include, e.g., cyclopropyl, cyclobutyl, cyclopentyl, cyclohexyl, cycloheptyl, and cyclooctyl. Polycyclic cycloalkyl radicals include, for example, adamantyl, norbornyl (i.e., bicyclo[2.2.1]heptanyl), norbornenyl, decalinyl, 7,7-dimethyl-bicyclo[2.2.1]heptanyl, and the like. Cycloalkyl may be optionally substituted by one or more substituents such as those substituents described herein.

“Cycloalkenyl” refers to a stable unsaturated non-aromatic monocyclic or polycyclic hydrocarbon radical consisting solely of carbon and hydrogen atoms, which includes fused or bridged ring systems, preferably having from three to twelve carbon atoms and comprising at least one double bond (i.e., C₃₋₁₂ cycloalkenyl). In certain embodiments, a cycloalkenyl comprises three to ten carbon atoms (i.e., C₃₋₁₀ cycloalkenyl). In other embodiments, a cycloalkenyl comprises five to seven carbon atoms (i.e., C₅₋₇ cycloalkenyl). The cycloalkenyl may be attached to the rest of the molecule by a single bond. Examples of monocyclic cycloalkenyls include, e.g., cyclopentenyl, cyclohexenyl, cycloheptenyl, and cyclooctenyl. Cycloalkenyl may be optionally substituted by one or more substituents such as those substituents described herein.

“Aryl” refers to a radical derived from an aromatic monocyclic or aromatic multicyclic hydrocarbon ring system by removing a hydrogen atom from a ring carbon atom. The aromatic monocyclic or aromatic multicyclic hydrocarbon ring system contains only hydrogen and carbon and from five to eighteen carbon atoms, where at least one of the rings in the ring system is aromatic, i.e., it contains a cyclic, delocalized (4n+2) x-electron system in accordance with the Hückel theory. The ring system from which aryl groups are derived include, but are not limited to, groups such as benzene, fluorene, indane, indene, tetralin and naphthalene. Aryl may be optionally substituted by one or more substituents such as those substituents described herein.

A “C_{x-y} carbocycle” is meant to include groups that contain from x to y carbons in a ring. For example, the term “C₃₋₆ carbocycle” can be a saturated, unsaturated or aromatic ring system that contains from 3 to 6 carbon atoms-any of which is optionally substituted as provided herein.

The term “heterocycle” as used herein refers to a saturated, unsaturated, non-aromatic or aromatic ring comprising one or more heteroatoms. Exemplary heteroatoms include N, O, Si, P, B, and S atoms. Heterocycles include 3- to 10-membered monocyclic rings and 6- to 12-membered bicyclic rings. Each ring of a bicyclic heterocycle may be selected from saturated, unsaturated, and aromatic rings. In some embodiments, the heterocycle comprises at least one heteroatom selected from oxygen, nitrogen, sulfur, or any combination thereof. In some embodiments, the heterocycle comprises at least one heteroatom selected from oxygen, nitrogen, or any combination thereof. In some embodiments, the heterocycle comprises at least one heteroatom selected from oxygen, sulfur, or any combination thereof. In some embodiments, the heterocycle comprises at least one heteroatom selected from nitrogen, sulfur, or any combination thereof. The heterocycle may be attached to the rest of the molecule through any atom of the heterocycle, valence permitting, such as a carbon or nitrogen atom of the heterocycle. In some embodiments, the heterocycle is a heteroaryl. In some embodiments, the heterocycle is a heterocycloalkyl. Exemplary heterocycles include pyrrolidinyl, pyrrolyl, imidazolyl, pyrazolyl, triazolyl, piperidinyl, pyridinyl, pyrimidinyl, pyridazinyl, pyrazinyl, thiophenyl, oxazolyl, thiazolyl, morpholinyl, indazolyl, indolyl, and quinolinyl. Heterocycle may be optionally substituted by one or more substituents such as those substituents described herein. Bicyclic heterocycles may be fused, bridged or spiro-ring systems. In an exemplary embodiment, a heterocycle, e.g., pyridyl, may be fused to a saturated or unsaturated ring, e.g., cyclohexane, cyclopentane, or cyclohexene. Heterocycle may be optionally substituted by one or more substituents such as those substituents described herein.

“Heterocycloalkyl” refers to a stable 3- to 12-membered non-aromatic ring radical that comprises two to twelve carbon atoms and at least one heteroatom wherein each heteroatom may be selected from N, O, Si, P, B, and S atoms. In some embodiments, the heterocycloalkyl comprises at least one heteroatom selected from oxygen, nitrogen, sulfur, or any combination thereof. In some embodiments, the heterocycloalkyl comprises at least one heteroatom selected from oxygen, nitrogen, or any combination thereof. In some embodiments, the heterocycloalkyl comprises at least one heteroatom selected from oxygen, sulfur, or any combination thereof. In some embodiments, the heterocycloalkyl comprises at least one heteroatom selected from nitrogen, sulfur, or any combination thereof. The heterocycloalkyl may be selected from monocyclic or bicyclic, and fused or bridged ring systems. The heteroatoms in the heterocycloalkyl radical are optionally oxidized. One or more nitrogen atoms, if present, are optionally quaternized. The heterocycloalkyl radical is partially or fully saturated. The heterocycloalkyl is attached to the rest of the molecule through any atom of the heterocycloalkyl, valence permitting, such as any carbon or nitrogen atoms of the heterocycloalkyl. Examples of heterocycloalkyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Heterocycloalkyl may be optionally substituted by one or more substituents such as those substituents described herein.

The term “heteroaryl” refers to a radical derived from a 5- to 12-membered aromatic ring radical whose ring structure comprise at least one heteroatom, preferably between one to four heteroatoms. In some embodiments, the heteroaryl comprises at least one heteroatom selected from oxygen, nitrogen, sulfur, or any combination thereof. In some embodiments, the heteroaryl comprises at least one heteroatom selected from oxygen, nitrogen, or any combination thereof. In some embodiments, the heteroaryl comprises at least one heteroatom selected from oxygen, sulfur, or any combination thereof. In some embodiments, the heteroaryl comprises at least one heteroatom selected from nitrogen, sulfur, or any combination thereof. One or more nitrogen atoms, if present, are optionally quaternized. The heteroaryl may be attached to the rest of the molecule through any atom of the heteroaryl, valence permitting, such as a carbon or nitrogen atom of the heteroaryl.

As used herein, the heteroaryl ring may be selected from monocyclic or polycyclic (bicyclic and fused or bridged) systems rings wherein at least one of the rings in the ring system is aromatic, i.e., it contains a cyclic, delocalized (4n+2) x-electron system in accordance with the Hückel theory. Heteroaryl includes aromatic single ring structures, preferably 5- to 6-membered rings, whose ring structures include at least one heteroatom, preferably one to four heteroatoms, more preferably one or two heteroatoms. Heteroaryl groups include, for example, pyrrole, furan, thiophene, imidazole, oxazole, thiazole, pyrazole, pyridine, pyrazine, pyridazine, and pyrimidine, and the like. Heteroaryl may be optionally substituted by one or more substituents such as those substituents described herein. Heteroaryl also includes polycyclic ring systems having two or more rings in which two or more atoms are common to two adjoining rings wherein at least one of the rings is heteroaromatic, e.g., the other rings can be aromatic or non-aromatic carbocyclic, or heterocyclic.

An “X-membered heterocycle” refers to the number of endocylic atoms, i.e., X, in the ring. For example, a 5-membered heteroaryl ring or 5-membered aromatic heterocycle has 5 endocyclic atoms, e.g., triazole, oxazole, thiophene, etc.

“Alkoxy” refers to a radical bonded through an oxygen atom of the formula-O-alkyl, where alkyl is an alkyl chain as defined above.

“Halo” or “halogen” refers to halogen substituents such as bromo, chloro, fluoro and iodo substituents.

As used herein, the term “haloalkyl” or “haloalkane” refers to an alkyl radical, as defined above, that is substituted by one or more halogen radicals, for example, trifluoromethyl, dichloromethyl, bromomethyl, 2,2,2-trifluoroethyl, 1-fluoromethyl-2-fluoroethyl, and the like. In some embodiments, the alkyl part of the fluoroalkyl radical is optionally further substituted.

Examples of halogen substituted alkanes (“haloalkanes”) include halomethane (e.g., chloromethane, bromomethane, fluoromethane, iodomethane), di- and trihalomethane (e.g., trichloromethane, tribromomethane, trifluoromethane, triiodomethane), 1-haloethane, 2-haloethane, 1,2-dihaloethane, 1-halopropane, 2-halopropane, 3-halopropane, 1,2-dihalopropane, 1,3-dihalopropane, 2,3-dihalopropane, 1,2,3-trihalopropane, and any other suitable combinations of alkanes (or substituted alkanes) and halogens (e.g., Cl, Br, F, and I). When an alkyl group is substituted with more than one halogen radicals, each halogen may be independently selected for example, 1-chloro, 2-fluoroethane.

The term “substituted” refers to moieties having substituents replacing a hydrogen on one or more carbons or substitutable heteroatoms, e.g., an NH or NH₂ of a compound. It will be understood that “substitution” or “substituted with” includes the implicit proviso that such substitution is in accordance with permitted valence of the substituted atom and the substituent, and that the substitution results in a stable compound, i.e., a compound which does not spontaneously undergo transformation such as by rearrangement, cyclization, elimination, etc. In certain embodiments, substituted refers to moieties having substituents replacing two hydrogen atoms on the same carbon atom, such as substituting the two hydrogen atoms on a single carbon with an oxo, imino or thioxo group. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds.

In some embodiments, substituents may include any substituents described herein, for example: halogen, hydroxy, oxo (═O), thioxo (═S), cyano (—CN), nitro (—NO₂), imino (═N—H), oximo (═N—OH), hydrazino (═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)₂R^{a} (where t is 1 or 2), —R^{b}—S(O)₂R^{a} (where t is 1 or 2), —R^{b}—S(O)₂OR^{a} (where t is 1 or 2), and —R^{b}—S(O)₂N(R^{a})₂ (where t is 1 or 2); and alkyl, alkenyl, alkynyl, aryl, aralkyl, aralkenyl, aralkynyl, cycloalkyl, cycloalkylalkyl, heterocycloalkyl, heterocycloalkylalkyl, heteroaryl, and heteroarylalkyl any of which may be optionally substituted by alkyl, alkenyl, alkynyl, halogen, haloalkyl, haloalkenyl, haloalkynyl, oxo (═O), thioxo (═S), cyano (—CN), nitro (—NO₂), imino (═N—H), oximo (═N—OH), hydrazine (═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)₂R^{a} (where t is 1 or 2), —R^{b}—S(O)₂R^{a} (where t is 1 or 2), —R^{b}—S(O)₂OR^{a} (where t is 1 or 2) and —R^{b}—S(O)²N(R^{a})₂ (where t is 1 or 2); wherein each R^{a} is independently selected from hydrogen, alkyl, cycloalkyl, cycloalkylalkyl, aryl, aralkyl, heterocycloalkyl, heterocycloalkylalkyl, heteroaryl, or heteroarylalkyl, wherein each R^{a}, valence permitting, may be optionally substituted with alkyl, alkenyl, alkynyl, halogen, haloalkyl, haloalkenyl, haloalkynyl, oxo (═O), thioxo (═S), cyano (—CN), nitro (—NO₂), imino (═N—H), oximo (═N—OH), hydrazine (═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)₂R^{a} (where t is 1 or 2), —R^{b}—S(O)₂R^{a} (where t is 1 or 2), —R^{b}—S(O)₂OR^{a} (where t is 1 or 2) and —R^{b}—S(O)₂N(R^{a})₂ (where t is 1 or 2); and wherein each R^{b} is independently selected from a direct bond or a straight or branched alkylene, alkenylene, or alkynylene chain, and each R^{c} is a straight or branched alkylene, alkenylene or alkynylene chain. It will be understood by those skilled in the art that substituents can themselves be substituted, if appropriate.

The term “salt” or “pharmaceutically acceptable salt” refers to salts derived from a variety of organic and inorganic counter ions well known in the art. Pharmaceutically acceptable acid addition salts can be formed with inorganic acids and organic acids. Pharmaceutically acceptable base addition salts can be formed with inorganic and organic bases.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable excipient” or “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient.

The terms “subject,” “individual,” and “patient” may be used interchangeably and refer to humans, the as well as non-human mammals (e.g., non-human primates, canines, equines, felines, porcines, bovines, ungulates, lagomorphs, and the like). In various embodiments, the subject can be a human (e.g., adult male, adult female, adolescent male, adolescent female, male child, female child) under the care of a physician or other health worker in a hospital, as an outpatient, or other clinical context. In certain embodiments, the subject may not be under the care or prescription of a physician or other health worker.

As used herein, the phrase “a subject in need thereof” refers to a subject, as described infra, that suffers from, or is at risk for, a pathology to be prophylactically or therapeutically treated with a compound or salt described herein.

The terms “administer”, “administered”, “administers” and “administering” are defined as providing a composition to a subject via a route known in the art, including but not limited to intravenous, intraarterial, oral, parenteral, buccal, topical, transdermal, rectal, intramuscular, subcutaneous, intraosseous, transmucosal, or intraperitoneal routes of administration. In certain embodiments, oral routes of administering a composition can be used. The terms “administer”, “administered”, “administers” and “administering” a compound should be understood to mean providing a compound of the invention or a prodrug of a compound of the invention to the individual in need.

As used herein, “treatment” or “treating” refers to an approach for obtaining beneficial or desired results with respect to a disease, disorder, or medical condition including, but not limited to, a therapeutic benefit and/or a prophylactic benefit. In certain embodiments, treatment or treating involves administering a compound or composition disclosed herein to a subject. A therapeutic benefit may include the eradication or amelioration of the underlying disorder being treated. Also, a therapeutic benefit may be achieved with the eradication or amelioration of one or more of the physiological symptoms associated with the underlying disorder, such as observing an improvement in the subject, notwithstanding that the subject may still be afflicted with the underlying disorder. In certain embodiments, for prophylactic benefit, the compositions are administered to a subject at risk of developing a particular disease, or to a subject reporting one or more of the physiological symptoms of a disease, even though a diagnosis of this disease may not have been made. Treating can include, for example, reducing, delaying or alleviating the severity of one or more symptoms of the disease or condition, or it can include reducing the frequency with which symptoms of a disease, defect, disorder, or adverse condition, and the like, are experienced by a patient. Treating can be used herein to refer to a method that results in some level of treatment or amelioration of the disease or condition, and can contemplate a range of results directed to that end, including but not restricted to prevention of the condition entirely.

In certain embodiments, the term “prevent” or “preventing” as related to a disease or disorder may refer to a compound that, in a statistical sample, reduces the occurrence of the disorder or condition in the treated sample relative to an untreated control sample, or delays the onset or reduces the severity of one or more symptoms of the disorder or condition relative to the untreated control sample.

A “therapeutic effect,” as that term is used herein, encompasses a therapeutic benefit and/or a prophylactic benefit as described above. A prophylactic effect includes delaying or eliminating the appearance of a disease or condition, delaying or eliminating the onset of symptoms of a disease or condition, slowing, halting, or reversing the progression of a disease or condition, or any combination thereof.

### Compounds

In some aspects, the present disclosure provides a compound represented by the structure of Formula (I):

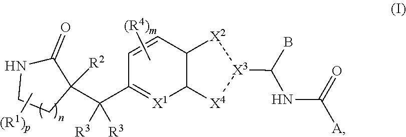

or a pharmaceutically acceptable salt thereof wherein:
A is selected from 5- to 6-membered heteroaryl and C₃₋₆ carbocycle, any of which is optionally substituted with one or more substituents independently selected from:
   halogen, —OR¹¹, —SR¹¹, —N(R¹¹)₂, —C(O)R¹¹, —C(O)N(R¹¹)₂, —N(R¹¹)C(O)R¹¹, —N(R¹¹)S(O)₂R¹¹, —C(O)OR¹¹, —OC(O)R¹¹, —S(O)R¹¹, —S(O)₂R¹¹, —NO₂, —CN; and
   C₁₋₁₀ alkyl optionally substituted with one or more substituents independently selected from halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —S(O)R¹¹, —S(O)₂R¹¹, —NO₂, ═O, ═S, ═N(R¹¹), —CN, C₃₋₁₀ carbocycle and 3- to 10-membered heterocycle; wherein the C₃₋₁₀ carbocycle and 3- to 10-membered heterocycle are each optionally substituted with one or more substituents selected from: halogen, —OR¹¹, —N(R¹¹)₂, —C(O)R¹¹, —C(O)N(R¹¹)₂, —N(R¹¹)C(O)R¹¹, —C(O)OR¹¹, —OC(O)R¹¹, —NO₂, ═O, ═N(R¹¹), and —CN;
B is selected from —C(H)(R⁵)₂ and C₃₋₁₀ carbocycle, wherein each C₃₋₁₀ carbocycle is optionally substituted with one or more substituents independently selected from:
   halogen, —OR¹², —SR¹², —N(R¹²)₂, —C(O)R¹², —C(O)N(R¹²)₂, —N(R¹²)C(O)R¹², —N(R¹²)S(O)₂R¹², —C(O)OR¹², —OC(O)R¹², —S(O)R¹², —S(O)₂R¹², —NO₂, ═O, ═S, ═N(R¹²), —CN;
   C₁₋₁₀ alkyl and C₃₋₁₀ carbocycle, any of which is optionally substituted with one or more substituents independently selected from halogen, —OR¹², —SR¹², —N(R¹²)₂, —C(O)R¹², —C(O)N(R¹²)₂, —N(R¹²)C(O)R¹², —C(O)OR¹², —OC(O)R¹², —S(O)R¹², —S(O)₂R¹², —NO₂, ═O, ═S, ═N(R¹²), —CN, C₃₋₁₀ carbocycle and 3- to 10-membered heterocycle; wherein the C₃₋₁₀ carbocycle and 3- to 10-membered heterocycle are each optionally substituted with one or more substituents selected from: halogen, —OR¹², —N(R¹²)₂, —C(O)R¹², —C(O)N(R¹²)₂, —N(R¹²)C(O)R¹², —C(O)OR¹², —OC(O)R¹², —NO₂, ═O, ═N(R¹²), and —CN;
each R⁵ is independently selected at each occurrence from (i), (ii), and (iii):
   (i) halogen, —OR¹³, —SR¹³, —N(R¹³)₂, —C(O)R¹³, —C(O)N(R¹³)₂, —N(R¹³) C(O)R¹³, —N(R¹³)S(O)₂R¹³, —C(O)OR¹³, —OC(O)R¹³, —S(O)R¹³, —S(O)₂R¹³, —NO₂, ═O, ═S, ═N(R¹³), —CN;
   (ii) C₁₋₁₀ alkyl optionally substituted with one or more substituents independently selected from halogen, —OR¹³, —SR¹³, —N(R¹³)₂, —C(O)R¹³, —C(O)N(R¹³)₂, —N(R¹³) C(O)R¹³, —N(R¹³)S(O)₂R¹³, —C(O)OR¹³, —OC(O)R¹³, —S(O)R¹³, —S(O)₂R¹³, —NO₂, ═O, ═S, ═N(R¹³), —CN; and
   (iii) C₃₋₁₀ carbocycle optionally substituted with one or more substituents independently selected from:
      halogen, —OR¹³, —SR¹³, —N(R¹³)₂, —C(O)R¹³, —C(O)N(R¹³)₂, —N(R¹³) C(O)R¹³, —N(R¹³)S(O)₂R¹³, —C(O)OR¹³, —OC(O)R¹³, —S(O)R¹³, —S(O)₂R¹³, —NO₂, ═O, ═S, ═N(R¹³), —CN; and
      C₁₋₁₀ alkyl optionally substituted with one or more substituents independently selected from halogen, —OR¹³, —SR¹³, —N(R¹³)₂, —C(O)R¹³, —C(O)N(R¹³)₂, —N(R¹³) C(O)R¹³, —C(O)OR¹³, —OC(O)R¹³, —S(O)R¹³, —S(O)₂R¹³, —NO₂, ═O, ═S, ═N(R¹³), and —CN;
each R¹ is independently selected at each occurrence from halogen, —OR¹⁴, —SR¹⁴, —N(R¹⁴)₂, —C(O)R¹⁴, —C(O)N(R¹⁴)₂, —N(R¹⁴)C(O)R¹⁴, —N(R¹⁴)S(O)₂R¹⁴, —C(O)OR¹⁴, —OC(O)R¹⁴, —S(O)R¹⁴, —S(O)₂R¹⁴, —NO₂, ═O, ═S, ═N(R¹⁴), —CN; and C₁₋₁₀ alkyl optionally substituted with one or more substituents independently selected from halogen, —OR¹⁴, —SR¹⁴, —N(R¹⁴)₂, —C(O)R¹⁴, —C(O)N(R¹⁴)₂, —N(R¹⁴)C(O)R¹⁴, —C(O)OR¹⁴, —OC(O)R¹⁴, —S(O)R¹⁴, —S(O)₂R¹⁴, —NO₂, ═O, ═S, ═N(R¹⁴), and —CN; or
   one R¹ on each of two adjacent carbons come together to form an optionally substituted C₃₋₆ carbocycle together with carbons atoms to which they are attached, wherein the C₃₋₆ carbocycle is optionally substituted with one or more substituents independently selected from halogen, —OR¹⁹, —SR¹⁹, —N(R¹⁹)₂, —C(O)R¹⁹, —C(O)N(R¹⁹)₂, —N(R¹⁹)C(O)R¹⁹, —N(R¹⁹)S(O)₂R¹⁹, —C(O)OR¹⁹, —OC(O)R¹⁹, —S(O)R¹⁹, —S(O)₂R¹⁹, —NO₂, ═O, ═S, ═N(R¹⁹), —CN, C₁₋₆ alkyl, and C₁₋₆ haloalkyl;
R² is selected from hydrogen, halogen, —OR¹⁵, —SR¹⁵, —N(R¹⁵)₂, —C(O)R¹⁵, —C(O)N(R¹⁵)₂, —N(R¹⁵)C(O)R¹⁵, —N(R¹⁵) S(O)₂R¹⁵, —C(O)OR¹⁵, —OC(O)R¹⁵, —S(O)R¹⁵, —S(O)₂R¹⁵, —NO₂, —CN; and C₁₋₆ alkyl optionally substituted with one or more substituents independently selected from halogen, —OR¹⁵, —SR¹⁵, —N(R¹⁵)₂, —C(O)R¹⁵, —C(O)N(R¹⁵)₂, —N(R¹⁵)C(O)R¹⁵, —C(O)OR¹⁵, —OC(O)R¹⁵, —S(O)R¹⁵, —S(O)₂R¹⁵, —NO₂, ═O, ═S, ═N(R¹⁵), and —CN;
each R³ is independently selected at each occurrence from hydrogen, halogen, —OR¹⁶, —SR¹⁶, —N(R¹⁶)₂, —C(O)R¹⁶, —C(O)N(R¹⁶)₂, —N(R¹⁶)C(O)R¹⁶, —N(R¹⁶)S(O)₂R¹⁶, —C(O)OR¹⁶, —OC(O)R¹⁶, —S(O)R¹⁶, —S(O)₂R¹⁶, —NO₂, —CN; and C₁₋₆ alkyl optionally substituted with one or more substituents independently selected from halogen, —OR¹⁶, —SR¹⁶, —N(R¹⁶)₂, —C(O)R¹⁶, —C(O)N(R¹⁶)₂, —N(R¹⁶)C(O)R¹⁶, —C(O)OR¹⁶, —OC(O)R¹⁶, —S(O)R¹⁶, —S(O)₂R¹⁶, —NO₂, ═O, ═S, ═N(R¹⁶), and —CN; or
R² and one R³ may come together to form C₃₋₆ carbocycle optionally substituted with one or more substituents independently selected from halogen, —OR²⁰, —SR²⁰, —N(R²⁰)₂, —C(O)R²⁰, —C(O)N(R²⁰)₂, —N(R²⁰)C(O)R²⁰, —N(R²⁰)S(O)₂R²⁰, —C(O)OR²⁰, —OC(O)R²⁰, —S(O)R²⁰, —S(O)₂R²⁰, —NO₂, ═O, ═S, ═N(R¹⁹), —CN, C₁₋₆ alkyl, and C₁₋₆ haloalkyl;
each R⁴ is independently selected at each occurrence from halogen, —OR¹⁷, —SR¹⁷, —N(R¹⁷)₂, —C(O)R¹⁷, —C(O)N(R¹⁷)₂, —N(R¹⁷)C(O)R¹⁷, —N(R¹⁷)S(O)₂R¹⁷, —C(O)OR¹⁷, —OC(O)R¹⁷, —S(O)R¹⁷, —S(O)₂R¹⁷, —NO₂, —CN; and C₁₋₁₀ alkyl optionally substituted with one or more substituents independently selected from halogen, —OR¹⁷, —SR¹⁷, —N(R¹⁷)₂, —C(O)R¹⁷, —C(O)N(R¹⁷)₂, —N(R¹⁷)C(O)R¹⁷, —C(O)OR¹⁷, —OC(O)R¹⁷, —S(O)R¹⁷, —S(O)₂R¹⁷, —NO₂, ═O, ═S, ═N(R¹⁷), and —CN;
X¹ is independently selected from N and C(R¹⁰);
X³ is C;
each X² and X⁴ is independently selected from N, N(H) and C(R¹⁰), wherein
   when X² is N(H), X⁴ is selected from N and C(R¹⁰); the bond between X² and X³ is a single bond and the bond between X³ and X⁴ is a double bond; or
   when X⁴ is N(H), X² is selected from N and C(R¹⁰); the bond between X² and X³ is a double bond and the bond between X³ and X⁴ is a single bond;
   wherein at least one of X² and X⁴ is N(H);
R¹⁰ is selected from hydrogen, halogen, —OR¹⁸, —SR¹⁸, —N(R¹⁸)₂, —C(O)R¹⁸, —C(O)OR¹⁸, —NO₂, —CN, C₁₋₆ alkyl, C₁₋₆ haloalkyl, —O—C₁₋₆ haloalkyl, and —O—C₁₋₆ alkyl;
R¹¹, R¹², R¹³, R¹⁴, R¹⁵, R¹⁶, R¹⁷, R¹⁸, R¹⁹, and R²⁰ are each independently selected at each occurrence from:
   hydrogen;
   C₁₋₆ alkyl optionally substituted with one more substituents independently selected from halogen, —O—C₁₋₆ alkyl, —O—C₁₋₆ haloalkyl, —NH₂, —NO₂, ═O, —CN, C₃₋₁₀ carbocycle and 3- to 10-membered heterocycle, wherein each C₃₋₁₀ carbocycle and 3- to 10-membered heterocycle are optionally substituted with one or more substituents independently selected from: halogen, —OH, —O—C₁₋₆ alkyl, —O—C₁₋₆ haloalkyl, —NH₂, —NO₂, ═O, and —CN; and
   C₃₋₁₀ carbocycle and 3- to 10-membered heterocycle optionally substituted with one or more substituents independently selected from: halogen, —OH, —O—C₁₋₆ alkyl, —O—C₁₋₆ haloalkyl, —NH₂, —NO₂, ═O, and —CN;
n is selected from 1 and 2;
m is selected from 0, 1 and 2; and
p is selected from 0, 1, 2, 3, 4, 5, and 6.

In some embodiments, for the compound or salt of Formula (I), n is 1.

In some embodiments, for the compound or salt of Formula (I), n is 2

In some embodiments, the structure of Formula (I) is represented by Formula (I-a) or Formula (I-b):

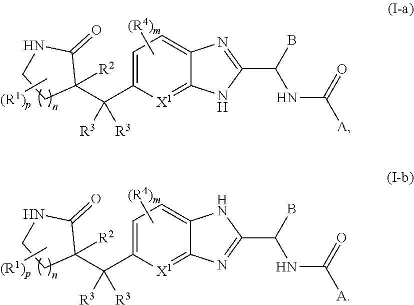

In some embodiments, for the compound or salt of Formula (I), X² is N(H), X⁴ is selected from N and C(R¹⁰); the bond between X² and X³ is a single bond and the bond between X³ and X⁴ is a double bond. In some embodiments, the structure of Formula (I) is represented by Formula (I-a):

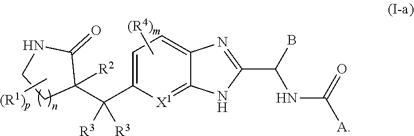

In some embodiments, for the compound or salt of Formula (I), when X⁴ is N(H), X² is selected from N and C(R¹⁰); the bond between X² and X³ is a double bond and the bond between X³ and X⁴ is a single bond. In some embodiments, the structure of Formula (I) is represented by Formula (I-b):

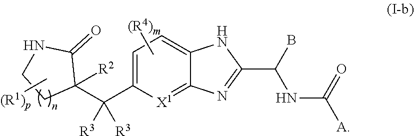

In some embodiments, for the compound or salt of Formula (I), (I-a), and (I-b), n is 1. In some embodiments, the structure of Formula (I) is represented by Formula (I-c) or (I-d):

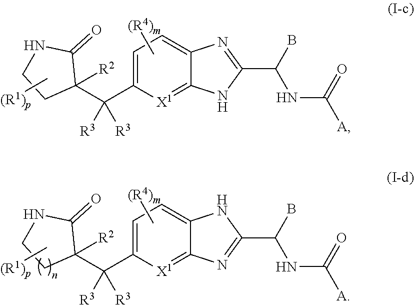

In some embodiments, for the compound or salt of Formula (I), (I-a), and (I-b), n is 2. In some embodiments, the structure of Formula (I) is represented by Formula (I-e) or (I-f):

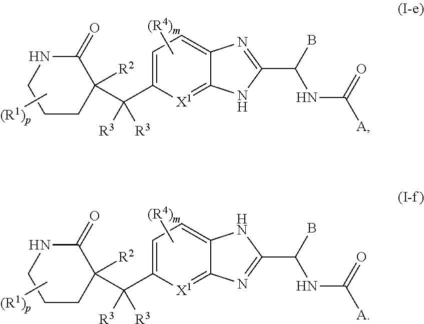

In some embodiments, for the compound or salt of Formula (I), (I-a), (I-b), (I-c), (I-d), (I-e), and (I-f), X¹ is N.

In some embodiments, for the compound or salt of Formula (I), (I-a), (I-b), (I-c), (I-d), (I-e), and (I-f), X¹ is C(R¹⁰). In some embodiments, R¹⁰ of C(R¹⁰) is selected from hydrogen, halogen, —OR¹⁸, C₁₋₆ alkyl, and C₁₋₆ haloalkyl. In some embodiments, R¹⁰ of C(R¹⁰) is hydrogen.

In some embodiments, for the compound or salt of Formula (I), (I-a), (I-b), (I-c), (I-d), (I-e), and (I-f), p is selected from 0, 1, 2, 3, 4, 5, and 6. In some embodiments, wherein p is selected from 1, 2, 3, 4, 5, and 6. In some embodiments, wherein p is selected from 0, 1, 2, 3, 4, and 5. In some embodiments, p is selected from 0, 1, 2, 3, and 4. In some embodiments, p is selected from 0, 1, 2, and 3. In some embodiments, p is selected from 0, 1, and 2. In some embodiments, p is selected from 0 and 1. In some embodiments, p is selected from 1, 2, 3, 4 and 5. In some embodiments, p is selected from 2, 3, 4 and 5. In some embodiments, p is selected from 3, 4 and 5. In some embodiments, p is selected from 4 and 5. In some embodiments, p is 0. In some embodiments, p is 1. In some embodiments, p is 2. In some embodiments, p is 3. In some embodiments, p is 4. In some embodiments, p is 5. In some embodiments, p is 6.

In some embodiments, for the compound or salt of Formula (I), (I-a), (I-b), (I-c), (I-d), (I-e), and (I-f), R¹ is selected from halogen, —OR¹⁴, —SR¹⁴, —N(R¹⁴)₂, —C(O)R¹⁴, —C(O)N(R¹⁴)₂, —N(R¹⁴)C(O)R¹⁴, —N(R¹⁴)S(O)₂R¹⁴, —C(O)OR¹⁴, —OC(O)R¹⁴, —S(O)R¹⁴, —S(O)₂R¹⁴, —NO₂, ═O, ═S, ═N(R¹⁴), —CN; and C₁₋₆ alkyl optionally substituted with one or more substituents independently selected from halogen, —OR¹⁴, —SR¹⁴, —N(R¹⁴)₂, —C(O)R¹⁴, —C(O)N(R¹⁴)₂, —N(R¹⁴)C(O)R¹⁴, —C(O)OR¹⁴, —OC(O)R¹⁴, —S(O)R¹⁴, —S(O)₂R¹⁴, —NO₂, ═O, ═S, ═N(R¹⁴), and —CN. In some embodiments, R¹ is selected from halogen, —OR¹⁴, —SR¹⁴, —N(R¹⁴)₂, —C(O)R¹⁴, —C(O)N(R¹⁴)₂, —N(R¹⁴)C(O)R¹⁴, —N(R¹⁴)S(O)₂R¹⁴, —C(O)OR¹⁴, —OC(O)R¹⁴, —S(O)R¹⁴, —S(O)₂R¹⁴, —NO₂, ═O, ═S, ═N(R¹⁴), —CN. In some embodiments, R¹ is selected from C₁₋₆ alkyl optionally substituted with one or more substituents independently selected from halogen, —OR¹⁴, —SR¹⁴, —N(R¹⁴)₂, —C(O)R¹⁴, —C(O)N(R¹⁴)₂, —N(R¹⁴)C(O)R¹⁴, —C(O)OR¹⁴, —OC(O)R¹⁴, —S(O)R¹⁴, —S(O)₂R¹⁴, —NO₂, ═O, ═S, ═N(R¹⁴), and —CN. In some embodiments, R¹ is selected from halogen, —OR¹⁴, —N(R¹⁴)₂, —NO₂, —CN, C₁₋₆ alkyl, and C₁₋₆ haloalkyl. In some embodiments, R¹ is selected from hydrogen and —CF₃.

In some embodiments, for the compound or salt of Formula (I), (I-a), (I-b), (I-c), (I-d), (I-e), and (I-f), one R¹ on each of two adjacent carbons come together to form an optionally substituted C₃₋₆ carbocycle together with carbons atoms to which they are attached, wherein the C₃₋₆ carbocycle is optionally substituted with one or more substituents independently selected from halogen, —OR¹⁹, —N(R¹⁹)₂, —NO₂, —CN, C₁₋₆ alkyl, and C₁₋₆ haloalkyl.

In some embodiments, for the compound or salt of Formula (I), (I-a), (I-b), (I-c), (I-d), (I-e), and (I-f), R² is selected from hydrogen, halogen, —OR¹⁵, —SR¹⁵, —N(R¹⁵)₂, —C(O)R¹⁵, —C(O)N(R¹⁵)₂, —N(R¹⁵)C(O)R¹⁵, —N(R¹⁵) S(O)₂R¹⁵, —C(O)OR¹⁵, —OC(O)R¹⁵, —S(O)R¹⁵, —S(O)₂R¹⁵, —NO₂, —CN, and C₁₋₆ alkyl optionally substituted with one or more substituents independently selected from halogen, —OR¹⁵, —SR¹⁵, —N(R¹⁵)₂, —C(O)R¹⁵, —C(O)N(R¹⁵)₂, —N(R¹⁵)C(O)R¹⁵, —C(O)OR¹⁵, —OC(O)R¹⁵, —S(O)R¹⁵, —S(O)₂R¹⁵, —NO₂, ═O, ═S, ═N(R¹⁵), and —CN. In some embodiments, R² is selected from hydrogen, halogen, —OR¹⁵, —SR¹⁵, —N(R¹⁵)₂, —C(O)R¹⁵, —C(O)N(R¹⁵)₂, —N(R¹⁵)C(O)R¹⁵, —N(R¹⁵) S(O)₂R¹⁵, —C(O)OR¹⁵, —OC(O)R¹⁵, —S(O)R¹⁵, —S(O)₂R¹⁵, —NO₂, —CN. In some embodiments, R² is selected from C₁₋₆ alkyl optionally substituted with one or more substituents independently selected from halogen, —OR¹⁵, —SR¹⁵, —N(R¹⁵)₂, —C(O)R¹⁵, —C(O)N(R¹⁵)₂, —N(R¹⁵)C(O)R¹⁵, —C(O)OR¹⁵, —OC(O)R¹⁵, —S(O)R¹⁵, —S(O)₂R¹⁵, —NO₂, ═O, ═S, ═N(R¹⁵), and —CN. In some embodiments, R² is selected from hydrogen, halogen, —OR¹⁵, —N(R¹⁵)₂, —C(O)R¹⁵, —NO₂, —CN, C₁₋₃ alkyl, and C₁₋₃ haloalkyl. In some embodiments, R² is selected from hydrogen, halogen, and C₁₋₃ alkyl. In some embodiments, R² is selected from hydrogen, halogen, methyl, and ethyl. In some embodiments, R² is selected from hydrogen and methyl.

In some embodiments, for the compound or salt of Formula (I), (I-a), (I-b), (I-c), (I-d), (I-e), and (I-f), each R³ is independently selected at each occurrence from hydrogen, halogen, —OR¹⁶, —SR¹⁶, —N(R¹⁶)₂, —C(O)R¹⁶, —C(O)N(R¹⁶)₂, —N(R¹⁶)C(O)R¹⁶, —N(R¹⁶)S(O)₂R¹⁶, —C(O)OR¹⁶, —OC(O)R¹⁶, —S(O)R¹⁶, —S(O)₂R¹⁶, —NO₂, —CN, and C₁₋₆ alkyl optionally substituted with one or more substituents independently selected from halogen, —OR¹⁶, —SR¹⁶, —N(R¹⁶)₂, —C(O)R¹⁶, and —C(O)N(R¹⁶)₂, —N(R¹⁶)C(O)R¹⁶, —C(O)OR¹⁶, —OC(O)R¹⁶, —S(O)R¹⁶, —S(O)₂R¹⁶, —NO₂, ═O, ═S, ═N(R¹⁶), and —CN. In some embodiments, each R³ is independently selected at each occurrence from hydrogen, halogen, —OR¹⁶, —SR¹⁶, —N(R¹⁶)₂, —C(O)R¹⁶, —C(O)N(R¹⁶)₂, —N(R¹⁶)C(O)R¹⁶, —N(R¹⁶)S(O)₂R¹⁶, —C(O)OR¹⁶, —OC(O)R¹⁶, —S(O)R¹⁶, —S(O)₂R¹⁶, —NO₂, and —CN. In some embodiments, each R³ is independently selected at each occurrence from C₁₋₆ alkyl optionally substituted with one or more substituents independently selected from halogen, —OR¹⁶, —SR¹⁶, —N(R¹⁶)₂, —C(O)R¹⁶, and —C(O)N(R¹⁶)₂, —N(R¹⁶)C(O)R¹⁶, —C(O)OR¹⁶, —OC(O)R¹⁶, —S(O)R¹⁶, —S(O)₂R¹⁶, —NO₂, ═O, ═S, ═N(R¹⁶), and —CN. In some embodiments, each R³ is independently selected at each occurrence from hydrogen and C₁₋₆ alkyl optionally substituted with one or more substituents independently selected from halogen, —OR¹⁶, —SR¹⁶, —N(R¹⁶)₂, —C(O)R¹⁶, and —C(O)N(R¹⁶)₂, —N(R¹⁶)C(O)R¹⁶, —C(O)OR¹⁶, —OC(O)R¹⁶, —S(O)R¹⁶, —S(O)₂R¹⁶, —NO₂, ═O, —S, —N(R¹⁶), and —CN. In some embodiments, each R³ is independently selected at each occurrence from hydrogen, halogen, —OR¹⁶, —N(R¹⁶)₂, —NO₂, —CN, C₁₋₃ alkyl, C₁₋₃ alkyl-OR¹⁶, and C₁₋₃ haloalkyl. In some embodiments, each R³ is independently selected at each occurrence from hydrogen and methyl.

In some embodiments, for the compound or salt of Formula (I), (I-a), (I-b), (I-c), (I-d), (I-e), and (I-f), one R³ is hydrogen and the other R³ is selected from halogen, —OR¹⁶, —SR¹⁶, —N(R¹⁶)₂, —C(O)R¹⁶, —C(O)N(R¹⁶)₂, —N(R¹⁶)C(O)R¹⁶, —N(R¹⁶)S(O)₂R¹⁶, —C(O)OR¹⁶, —OC(O)R¹⁶, —S(O)R¹⁶, —S(O)₂R¹⁶, —NO₂, —CN; and C₁₋₆ alkyl optionally substituted with one or more substituents independently selected from halogen, —OR¹⁶, —SR¹⁶, —N(R¹⁶)₂, —C(O)R¹⁶, —C(O)N(R¹⁶)₂, —N(R¹⁶)C(O)R¹⁶, —C(O)OR¹⁶, —OC(O)R¹⁶, —S(O)R¹⁶, —S(O)₂R¹⁶, —NO₂, ═O, ═S, ═N(R¹⁶), and —CN. In some embodiments, one R³ is hydrogen and the other R³ is selected from halogen, —OR¹⁶, —SR¹⁶, —N(R¹⁶)₂, —C(O)R¹⁶, —C(O)N(R¹⁶)₂, —N(R¹⁶)C(O)R¹⁶, —N(R¹⁶)S(O)₂R¹⁶, —C(O)OR¹⁶, —OC(O)R¹⁶, —S(O)R¹⁶, —S(O)₂R¹⁶, —NO₂, and —CN. In some embodiments, one R³ is hydrogen and the other R³ is selected from halogen, —OR¹⁶, —N(R¹⁶)₂, —NO₂, —CN, C₁₋₃ alkyl, C₁₋₃ alkyl-OR¹⁶, and C₁₋₃ haloalkyl. In some embodiments, one R³ is hydrogen and the other R³ is methyl. In some embodiments, each R² is methyl. In some embodiments, each R³ is hydrogen.

In some embodiments, for the compound or salt of Formula (I), (I-a), (I-b), (I-c), (I-d), (I-e), and (I-f), m is 2.

In some embodiments, for the compound or salt of Formula (I), (I-a), (I-b), (I-c), (I-d), (I-e), and (I-f), m is 1.

In some embodiments, for the compound or salt of Formula (I), (I-a), (I-b), (I-c), (I-d), (I-e), and (I-f), m is 0.

In some embodiments, for the compound or salt of Formula (I), (I-a), (I-b), (I-c), (I-d), (I-e), and (I-f), each R⁴ is independently selected from at each occurrence from halogen, —OR¹⁷, —SR¹⁷, —N(R¹⁷)₂, —C(O)R¹⁷, —C(O)N(R¹⁷)₂, —N(R¹⁷)C(O)R¹⁷, —N(R¹⁷)S(O)₂R¹⁷, —C(O)OR¹⁷, —OC(O)R¹⁷, —S(O)R¹⁷, —S(O)₂R¹⁷, —NO₂, —CN, and C₁₋₆ alkyl optionally substituted with one or more substituents independently selected from halogen, —OR¹⁷, —SR¹⁷, —N(R¹⁷)₂, —C(O)R¹⁷, —C(O)N(R¹⁷)₂, —N(R¹⁷)C(O)R¹⁷, —C(O)OR¹⁷, —OC(O)R¹⁷, —S(O)R¹⁷, —S(O)₂R¹⁷, —NO₂, ═O, ═S, —N(R¹⁷), and —CN. In some embodiments, each R⁴ is independently selected from at each occurrence from halogen, —OR¹⁷, —SR¹⁷, —N(R¹⁷)₂, —C(O)R¹⁷, —C(O)N(R¹⁷)₂, —N(R¹⁷)C(O)R¹⁷, —N(R¹⁷)S(O)₂R¹⁷, —C(O)OR¹⁷, —OC(O)R¹⁷, —S(O)R¹⁷, —S(O)₂R¹⁷, —NO₂, and —CN. In some embodiments, each R⁴ is independently selected from at each occurrence from C₁₋₁₀ alkyl optionally substituted with one or more substituents independently selected from halogen, —OR¹⁷, —SR¹⁷, —N(R¹⁷)₂, —C(O)R¹⁷, —C(O)N(R¹⁷)₂, —N(R¹⁷)C(O)R¹⁷, —C(O)OR¹⁷, —OC(O)R¹⁷, —S(O)R¹⁷, —S(O)₂R¹⁷, —NO₂, ═O, ═S, ═N(R¹⁷), and —CN. In some embodiments, each R⁴ is selected from halogen, —OR¹⁷, —N(R¹⁷)₂, —NO₂, —CN, C₁₋₃ alkyl, and C₁₋₃ haloalkyl. In some embodiments, each R⁴ is selected from chloro, fluoro, methyl, ethyl, and —CN. In some embodiments, R⁴ is selected from fluoro, methyl, and —CN. In some embodiments, R⁴ is fluoro. In some embodiments, R⁴ is methyl. In some embodiments, R⁴ is —CN.

In some embodiments, for the compound or salt of Formula (I), (I-a), (I-b), (I-c), (I-d), (I-e), and (I-f), m is 1 and R⁴ is selected from halogen, —OR¹⁷, —SR¹⁷, —N(R¹⁷)₂, —C(O)R¹⁷, —C(O)N(R¹⁷)₂, —N(R¹⁷)C(O)R¹⁷, —N(R¹⁷)S(O)₂R¹⁷, —C(O)OR¹⁷, —OC(O)R¹⁷, —S(O)R¹⁷, —S(O)₂R¹⁷, —NO₂, —CN; and C₁₋₁₀ alkyl optionally substituted with one or more substituents independently selected from halogen, —OR¹⁷, —SR¹⁷, —N(R¹⁷)₂, —C(O)R¹⁷, —C(O)N(R¹⁷)₂, —N(R¹⁷)C(O)R¹⁷, —C(O)OR¹⁷, —OC(O)R¹⁷, —S(O)R¹⁷, —S(O)₂R¹⁷, —NO₂, ═O, ═S, ═N(R¹⁷), and —CN. In some embodiments, m is 1 and R⁴ is selected from halogen, —OR¹⁷, —SR¹⁷, —N(R¹⁷)₂, —C(O)R¹⁷, —C(O)N(R¹⁷)₂, —N(R¹⁷)C(O)R¹⁷, —N(R¹⁷)S(O)₂R¹⁷, —C(O)OR¹⁷, —OC(O)R¹⁷, —S(O)R¹⁷, —S(O)₂R¹⁷, —NO₂, and —CN. In some embodiments, m is 1 and R⁴ is selected from C₁₋₁₀ alkyl optionally substituted with one or more substituents independently selected from halogen, —OR¹⁷, —SR¹⁷, —N(R¹⁷)₂, —C(O)R¹⁷, —C(O)N(R¹⁷)₂, —N(R¹⁷)C(O)R¹⁷, —C(O)OR¹⁷, —OC(O)R¹⁷, —S(O)R¹⁷, —S(O)₂R¹⁷, —NO₂, ═O, ═S, ═N(R¹⁷), and —CN. In some embodiments, m is 1 and R⁴ is selected from halogen, —OR¹⁷, —N(R¹⁷)₂, —NO₂, —CN, C₁₋₃ alkyl, and C₁₋₃ haloalkyl. In some embodiments, m is 1 and R⁴ is selected from halogen, —OR¹⁷, —N(R¹⁷)₂, —NO₂, —CN, C₁₋₃ alkyl, and C₁₋₃ haloalkyl. In some embodiments, m is 1 and R⁴ is selected from chloro, fluoro, methyl, ethyl, and —CN.

In some embodiments, for the compound or salt of Formula (I), (I-a), (I-b), (I-c), (I-d), (I-e), and (I-f), m is 2 and R⁴ is selected from halogen, —OR¹⁷, —SR¹⁷, —N(R¹⁷)₂, —C(O)R¹⁷, —C(O)N(R¹⁷)₂, —N(R¹⁷)C(O)R¹⁷, —N(R¹⁷)S(O)₂R¹⁷, —C(O)OR¹⁷, —OC(O)R¹⁷, —S(O)R¹⁷, —S(O)₂R¹⁷, —NO₂, —CN; and C₁₋₁₀ alkyl optionally substituted with one or more substituents independently selected from halogen, —OR¹⁷, —SR¹⁷, —N(R¹⁷)₂, —C(O)R¹⁷, —C(O)N(R¹⁷)₂, —N(R¹⁷)C(O)R¹⁷, —C(O)OR¹⁷, —OC(O)R¹⁷, —S(O)R¹⁷, —S(O)₂R¹⁷, —NO₂, ═O, ═S, ═N(R¹⁷), and —CN. In some embodiments, m is 2 and R⁴ is selected from halogen, —OR¹⁷, —SR¹⁷, —N(R¹⁷)₂, —C(O)R¹⁷, —C(O)N(R¹⁷)₂, —N(R¹⁷)C(O)R¹⁷, —N(R¹⁷)S(O)₂R¹⁷, —C(O)OR¹⁷, —OC(O)R¹⁷, —S(O)R¹⁷, —S(O)₂R¹⁷, —NO₂, and —CN. In some embodiments, m is 2 and R⁴ is selected from C₁₋₁₀ alkyl optionally substituted with one or more substituents independently selected from halogen, —OR¹⁷, —SR¹⁷, —N(R¹⁷)₂, —C(O)R¹⁷, —C(O)N(R¹⁷)₂, —N(R¹⁷)C(O)R¹⁷, —C(O)OR¹⁷, —OC(O)R¹⁷, —S(O)R¹⁷, —S(O)₂R¹⁷, —NO₂, ═O, —S, ═N(R¹⁷), and —CN. In some embodiments, m is 2 and R⁴ is selected from halogen, —OR¹⁷, —N(R¹⁷)₂, —NO₂, —CN, C₁₋₃ alkyl, and C₁₋₃ haloalkyl. In some embodiments, m is 2 and R⁴ is selected from halogen, —OR¹⁷, —N(R¹⁷)₂, —NO₂, —CN, C₁₋₃ alkyl, and C₁₋₃ haloalkyl. In some embodiments, m is 2 and R⁴ is selected from chloro, fluoro, methyl, ethyl, and —CN.

In some embodiments, for the compound or salt of Formula (I), (I-a), (I-b), (I-c), (I-d), (I-e), and (I-f), A is optionally substituted C₃₋₆ carbocycle. In some embodiments, A is optionally substituted saturated C₃₋₆ carbocycle. In some embodiments, A is optionally substituted unsaturated C₃₋₆ carbocycle. In some embodiments, A is selected from optionally substituted C₃₋₅ carbocycle, optionally substituted C₃₋₄ carbocycle, optionally substituted C₄₋₆ carbocycle, and optionally substituted C₅₋₆ carbocycle. In some embodiments, A is selected from optionally substituted C₃ carbocycle, optionally substituted C₄ carbocycle, optionally substituted C₅ carbocycle, and optionally substituted C₆ carbocycle.

In some embodiments, for the compound or salt of Formula (I), (I-a), (I-b), (I-c), (I-d), (I-e), and (I-f), A is optionally substituted 5- to 6-membered heteroaryl. In some embodiments, A is optionally substituted 5- to 6-membered heteroaryl comprising at least one heteroatom selected from nitrogen, oxygen, and sulfur. In some embodiments, A is optionally substituted 5- to 6-membered heteroaryl comprising at least one nitrogen or oxygen heteroatom. In some embodiments, A is optionally substituted 5- to 6-membered heteroaryl comprising at least one nitrogen or sulfur heteroatom. In some embodiments, A is optionally substituted 5- to 6-membered heteroaryl comprising at least one sulfur or oxygen heteroatom. In some embodiments, A is optionally substituted 5- to 6-membered heteroaryl comprising at least one sulfur heteroatom. In some embodiments, A is optionally substituted 5- to 6-membered heteroaryl comprising at least one oxygen heteroatom. In some embodiments, A is optionally substituted 5- to 6-membered heteroaryl comprising at least one nitrogen heteroatom.

In some embodiments, for the compound or salt of Formula (I), (I-a), (I-b), (I-c), (I-d), (I-e), and (I-f), A is a saturated C₃₋₆ carbocycle optionally substituted with one or more substituents independently selected from:
halogen, —OR¹¹, —SR¹¹, —N(R¹¹)₂, —C(O)R¹¹, —C(O)N(R¹¹)₂, —N(R¹¹)C(O)R¹¹, —N(R¹¹)S(O)₂R¹¹, —C(O)OR¹¹, —OC(O)R¹¹, —S(O)R¹¹, —S(O)₂R¹¹, —NO₂, —CN; and
C₁₋₆ alkyl optionally substituted with one or more substituents independently selected from halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —S(O)R¹¹, —S(O)₂R¹¹, —NO₂, ═O, ═S, ═N(R¹¹), —CN, C₃₋₁₀ carbocycle and 3- to 10-membered heterocycle; wherein the C₃₋₁₀ carbocycle and 3- to 10-membered heterocycle are each optionally substituted with one or more substituents selected from: halogen, —OR¹¹, —N(R¹¹)₂, —C(O)R¹¹, —C(O)N(R¹¹)₂, —N(R¹¹)C(O)R¹¹, —C(O)OR¹¹, —OC(O)R¹¹, —NO₂, ═O, ═N(R¹¹), and —CN.

In some embodiments, for the compound or salt of Formula (I), (I-a), (I-b), (I-c), (I-d), (I-e), (I-f), A is 5- to 6-membered heteroaryl, each of which is optionally substituted with one or more substituents independently selected from:
halogen, —OR¹¹, —SR¹¹, —N(R¹¹)₂, —C(O)R¹¹, —C(O)N(R¹¹)₂, —N(R¹¹)C(O)R¹¹, —N(R¹¹)S(O)₂R¹¹, —C(O)OR¹¹, —OC(O)R¹¹, —S(O)R¹¹, —S(O)₂R¹¹, —NO₂, —CN; and
C₁₋₆ alkyl optionally substituted with one or more substituents independently selected from halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —S(O)R¹¹, —S(O)₂R¹¹, —NO₂, ═O, ═S, ═N(R¹¹), —CN, C₃₋₁₀ carbocycle and 3- to 10-membered heterocycle; wherein the C₃₋₁₀ carbocycle and 3- to 10-membered heterocycle are each optionally substituted with one or more substituents selected from: halogen, —OR¹¹, —N(R¹¹)₂, —C(O)R¹¹, —C(O)N(R¹¹)₂, —N(R¹¹)C(O)R¹¹, —C(O)OR¹¹, —OC(O)R¹¹, —NO₂, ═O, ═N(R¹¹), and —CN.

In some embodiments, for the compound or salt of Formula (I), (I-a), (I-b), (I-c), (I-d), (I-e), (I-f), A is selected from a saturated C₃₋₆ carbocycle and a 5-membered heteroaryl, any of which is optionally substituted with one or more substituents independently selected from:
halogen, —OR¹¹, —SR¹¹, —N(R¹¹)₂, —C(O)R¹¹, —C(O)N(R¹¹)₂, —C(O)OR¹¹, —OC(O)R¹¹, —NO₂, —CN; and
C₁₋₆ alkyl optionally substituted with one or more substituents independently selected from halogen, —OR¹¹, —SR¹¹, —N(R¹¹)₂, —C(O)R¹¹, —C(O)N(R¹¹)₂, —C(O)OR¹¹, —OC(O)R¹¹, —NO₂, —CN, C₃₋₁₀ carbocycle and 3- to 10-membered heterocycle; wherein the C₃₋₁₀ carbocycle and 3- to 10-membered heterocycle are each optionally substituted with one or more substituents selected from: —OR¹¹, —SR¹¹, —N(R¹¹)₂, —C(O)R¹¹, —C(O)N(R¹¹)₂, —C(O)OR¹¹, —OC(O)R¹¹, —NO₂, and —CN.

In some embodiments, for the compound or salt of Formula (I), (I-a), (I-b), (I-c), (I-d), (I-e), and (I-f), A is saturated C₃₋₆ carbocycle optionally substituted with one or more substituents independently selected from: halogen, —OR¹¹, —SR¹¹, —N(R¹¹)₂, —C(O)R¹¹, —C(O)N(R¹¹)₂, —C(O)OR¹¹, —OC(O)R¹¹, —NO₂, and —CN. In some embodiments, A is saturated C₃₋₆ carbocycle optionally substituted with one or more C₁₋₆ alkyl optionally substituted with one or more substituents independently selected from halogen, —OR¹¹, —SR¹¹, —N(R¹¹)₂, —C(O)R¹¹, —C(O)N(R¹¹)₂, —C(O)OR¹¹, —OC(O)R¹¹, —NO₂, —CN, C₃₋₆ carbocycle and 3- to 6-membered heterocycle; wherein the C₃₋₆ carbocycle and 3- to 6-membered heterocycle are each optionally substituted with one or more substituents selected from: —OR¹¹, —SR¹¹, —N(R¹¹)₂, —C(O)R¹¹, —C(O)N(R¹¹)₂, —C(O)OR¹¹, —OC(O)R¹¹, —NO₂, and —CN.

In some embodiments, for the compound or salt of Formula (I), (I-a), (I-b), (I-c), (I-d), (I-e), and (I-f), A is saturated C₃₋₆ carbocycle optionally substituted with one or more substituents independently selected from halogen, —OR¹¹, —N(R¹¹)₂, —CN, C₁₋₃ alkyl, and C₁₋₃ haloalkyl. In some embodiments, A is selected from cyclopropyl, cyclobutyl, cyclopentyl, and cyclohexyl, any one of which is optionally substituted with one or more substituents independently selected from halogen, —OR¹¹, —N(R¹¹)₂, —CN, C₁₋₃ alkyl, and C₁₋₃ haloalkyl. In some embodiments, A is cyclopropyl optionally substituted with one or more substituents independently selected from halogen, C₁₋₃ alkyl, and C₁₋₃ haloalkyl. In some embodiments, A is

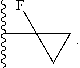 [SMILES: *C1(F)CC1]

In some embodiments, for the compound or salt of Formula (I), (I-a), (I-b), (I-c), (I-d), (I-e), and (I-f), A is 5- to 6-membered heteroaryl optionally substituted with one or more substituents independently selected from: halogen, —OR¹¹, —SR¹¹, —N(R¹¹)₂, —C(O)R¹¹, —C(O)N(R¹¹)₂, —C(O)OR¹¹, —OC(O)R¹¹, —NO₂, and —CN. In some embodiments, A is 5- to 6-membered heteroaryl optionally substituted with one or more substituents independently selected from: C₁₋₆ alkyl optionally substituted with one or more substituents independently selected from halogen, —OR¹¹, —SR¹¹, —N(R¹¹)₂, —C(O)R¹¹, —C(O)N(R¹¹)₂, —C(O)OR¹¹, —OC(O)R¹¹, —NO₂, —CN, C₃₋₆ carbocycle and 3- to 6-membered heterocycle; wherein the C₃₋₆ carbocycle and 3- to 6-membered heterocycle are each optionally substituted with one or more substituents selected from: —OR¹¹, —SR¹¹, —N(R¹¹)₂, —C(O)R¹¹, —C(O)N(R¹¹)₂, —C(O)OR¹¹, —OC(O)R¹¹, —NO₂, and —CN.

In some embodiments, for the compound or salt of Formula (I), (I-a), (I-b), (I-c), (I-d), (I-e), and (I-f), A is selected from pyrazolyl, imidazolyl, oxazolyl, isoxazolyl, oxadiazolyl, triazolyl, and tetrazolyl, any one of which is optionally substituted with one or more substituents independently selected from: C₁₋₆ alkyl optionally substituted with one or more substituents independently selected from halogen, —OR¹¹, —SR¹¹, —N(R¹¹)₂, —C(O)R¹¹, —C(O)N(R¹¹)₂, —C(O)OR¹¹, —OC(O)R¹¹, —NO₂, and —CN. In some embodiments, A is selected from pyrazolyl and oxadiazolyl, each of which is optionally substituted with C₁₋₆ alkyl optionally substituted with one or more substituents independently selected from halogen, —OR¹¹, —N(R¹¹)₂, —C(O)R¹¹, —NO₂, and —CN. In some embodiments, A is selected from pyrazolyl and oxadiazolyl, each of which is optionally substituted with methyl, ethyl, isopropyl, and propyl. In some embodiments, A is selected from:

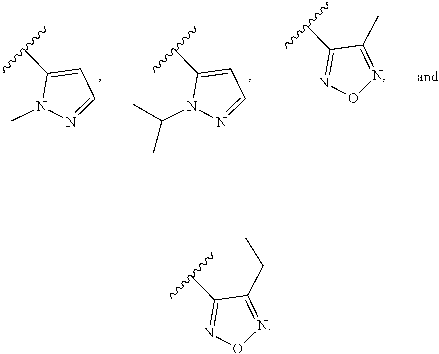

In some embodiments, A is selected from

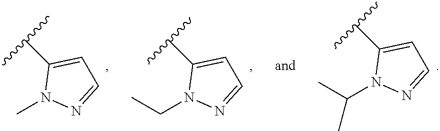

In some embodiments, A is

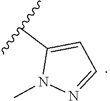 [SMILES: *c1ccnn1C]

In some embodiments, A is

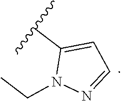 [SMILES: *c1ccnn1CC]

In some embodiments, A is

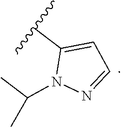 [SMILES: *c1ccnn1C(C)C]

In some embodiments, A is selected from

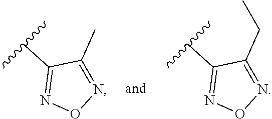

In some embodiments, A is

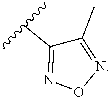 [SMILES: *c1nonc1C]

In some embodiments, A is

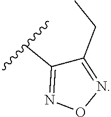 [SMILES: *c1nonc1CC]

In some embodiments, for the compound or salt of Formula (I), (I-a), (I-b), (I-c), (I-d), (I-e), and (I-f), B is —C(H)(R⁵)₂. In some embodiments, each R⁵ is independently selected at each occurrence from halogen, —OR¹³, —SR¹³, —N(R¹³)₂, —C(O)R¹³, —C(O)N(R¹³)₂, —N(R¹³) C(O)R¹³, —N(R¹³)S(O)₂R¹³, —C(O)OR¹³, —OC(O)R¹³, —S(O)R¹³, —S(O)₂R¹³, —NO₂, ═O, ═S, ═N(R¹³), and —CN. In some embodiments, each R⁵ is independently selected at each occurrence from C₁₋₆ alkyl optionally substituted with one or more substituents independently selected from halogen, —OR¹³, —SR¹³, —N(R¹³)₂, —C(O)R¹³, —C(O)N(R¹³)₂, —N(R¹³) C(O)R¹³, —N(R¹³)S(O)₂R¹³, —C(O)OR¹³, —OC(O)R¹³, —S(O)R¹³, —S(O)₂R¹³, —NO₂, ═O, ═S, ═N(R¹³), and —CN. In some embodiments, each R⁵ is independently selected at each occurrence from C₃₋₆ carbocycle optionally substituted with one or more substituents independently selected from halogen, —OR¹³, —SR¹³, —N(R¹³)₂, —C(O)R¹³, —C(O)N(R¹³)₂, —N(R¹³) C(O)R¹³, —N(R¹³)S(O)₂R¹³, —C(O)OR¹³, —OC(O)R¹³, —S(O)R¹³, —S(O)₂R¹³, —NO₂, ═O, ═S, ═N(R¹³), and —CN. In some embodiments, each R⁵ is independently selected at each occurrence from C₃₋₆ carbocycle optionally substituted with one or more substituents C₁₋₆ alkyl optionally substituted with one or more substituents independently selected from halogen, —OR¹³, —SR¹³, —N(R¹³)₂, —C(O)R¹³, —C(O)N(R¹³)₂, —N(R¹³) C(O)R¹³, —C(O)OR¹³, —OC(O)R¹³, —S(O)R¹³, —S(O)₂R¹³, —NO₂, ═O, ═S, ═N(R¹³), and —CN.

In some embodiments, for the compound or salt of Formula (I), (I-a), (I-b), (I-c), (I-d), (I-e), and (I-f), each R⁵ of —C(H)(R⁵)₂ is independently selected from:
halogen, —OR¹³, —N(R¹³)₂, —C(O)R¹³, —C(O)OR¹³, —NO₂, —CN;
C₁₋₆ alkyl optionally substituted with one or more substituents independently selected from halogen, —OR¹³, —N(R¹³)₂, —C(O)R¹³, —C(O)OR¹³, —NO₂, ═O, ═S, ═N(R¹³), and —CN; and
C₃₋₁₀ carbocycle optionally substituted with one or more substituents independently selected from halogen, —OR¹³, —N(R¹³)₂, —C(O)R¹³, —C(O)OR¹³, —NO₂, ═O, ═S, ═N(R¹³), —CN, C₁₋₆ alkyl, and C₁₋₆ haloalkyl.

In some embodiments, for the compound or salt of Formula (I), (I-a), (I-b), (I-c), (I-d), (I-e), and (I-f), each R⁵ of —C(H)(R⁵)₂ is independently selected from C₃₋₁₀ carbocycle optionally substituted with one or more substituents independently selected from halogen, —OR¹³, —N(R¹³)₂, —C(O)R¹³, —C(O)OR¹³, —NO₂, ═O, —S, ═N(R¹³), —CN, C₁₋₆ alkyl, and C₁₋₆ haloalkyl. In some embodiments, —CH(R⁵)₂ is selected from:

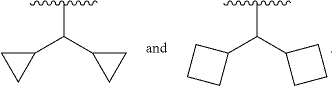 [SMILES: *C(C1CC1)C1CC1 ; *C(C1CCC1)C1CCC1]

In some embodiments, —CH(R⁵)₂ is

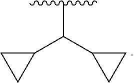 [SMILES: *C(C1CC1)C1CC1]

In some embodiments, CH(R⁵)₂ is

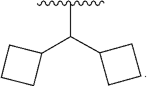 [SMILES: *C(C1CCC1)C1CCC1]

In some embodiments, for the compound or salt of Formula (I), (I-a), (I-b), (I-c), (I-d), (I-e), and (I-f), each R⁵ of —C(H)(R⁵)₂ is independently selected from:
halogen, —OR¹³, —N(R¹³)₂, —C(O)R¹³, —C(O)OR¹³, —NO₂, —CN; and
C₃₋₁₀ carbocycle optionally substituted with one or more substituents independently selected from halogen, —OR¹³, —N(R¹³)₂, —C(O)R¹³, —C(O)OR¹³, —NO₂, ═O, ═S, ═N(R¹³), —CN, C₁₋₆ alkyl, and C₁₋₆ haloalkyl. In some embodiments, —CH(R⁵)₂ is

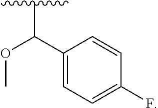 [SMILES: *C(OC)c1ccc(F)cc1]

In some embodiments, for the compound or salt of Formula (I), (I-a), (I-b), (I-c), (I-d), (I-e), and (I-f), each R⁵ of —C(H)(R⁵)₂ is independently selected from:
C₁₋₆ alkyl optionally substituted with one or more substituents independently selected from halogen, —OR¹³, —N(R¹³)₂, —C(O)R¹³, —C(O)OR¹³, —NO₂, ═O, ═S, ═N(R¹³), and —CN; and
C₃₋₁₀ carbocycle optionally substituted with one or more substituents independently selected from halogen, —OR¹³, —N(R¹³)₂, —C(O)R¹³, —C(O)OR¹³, —NO₂, ═O, ═S, ═N(R¹³), —CN, C₁₋₆ alkyl, and C₁₋₆ haloalkyl. In some embodiments, —CH(R⁵)₂ is selected from:

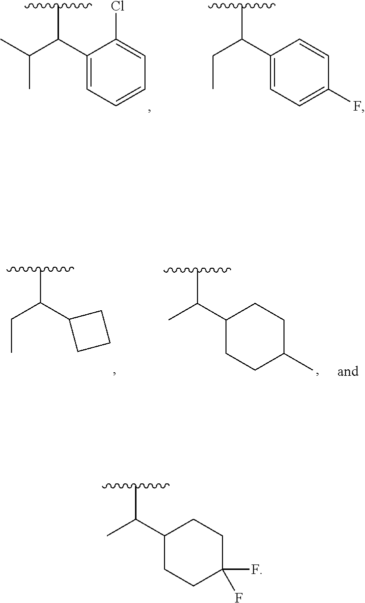

In some embodiments, —CH(R⁵)₂ is selected from:

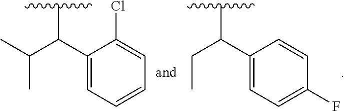 [SMILES: *C(c1ccccc1Cl)C(C)C ; *C(CC)c1ccc(F)cc1]

In some embodiments, —CH(R⁵)₂ is

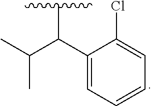 [SMILES: *C(c1ccccc1Cl)C(C)C]

In some embodiments, —CH(R⁵)₂ is

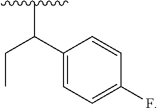 [SMILES: *C(CC)c1ccc(F)cc1]

In some embodiments, —CH(R⁵)₂ is selected from

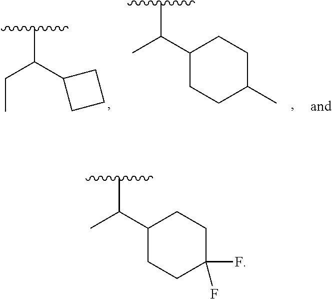

In some embodiments, —CH(R⁵)₂ is

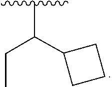 [SMILES: *C(CC)C1CCC1]

In some embodiments, —CH(R⁵)₂ is

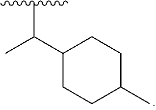 [SMILES: *C(C)C1CCC(C)CC1]

In some embodiments, —CH(R⁵)₂ is

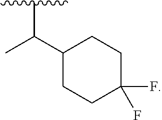 [SMILES: *C(C)C1CCC(F)(F)CC1]

In some embodiments, for the compound or salt of Formula (I), (I-a), (I-b), (I-c), (I-d), (I-e), and (I-f), B is optionally substituted C₃₋₁₀ carbocycle. In some embodiments, B is selected from optionally substituted C₃₋₄ carbocycle, optionally substituted C₃₋₅ carbocycle, optionally substituted C₃₋₆ carbocycle, optionally substituted C₃₋₇ carbocycle, optionally substituted C₃₋₈ carbocycle, and optionally substituted C₃₋₉ carbocycle. In some embodiments, B is selected from optionally substituted C₄₋₁₀ carbocycle, optionally substituted C₅₋₁₀ carbocycle, optionally substituted C₆₋₁₀ carbocycle, optionally substituted C₇₋₁₀ carbocycle, optionally substituted C₈₋₁₀ carbocycle, and optionally substituted C₉₋₁₀ carbocycle. In some embodiments, B is an optionally substituted C₃₋₁₀ saturated carbocycle. In some embodiments, B is an optionally substituted C₃₋₁₀ unsaturated carbocycle. In some embodiments, B is selected from an optionally substituted C₃₋₆ carbocycle and optionally substituted C₆₋₁₀ carbocycle. In some embodiments, B is selected from an optionally substituted C₃₋₈ monocyclic carbocycle, optionally substituted C₆₋₁₀ bicyclic carbocycle, and optionally substituted C₆₋₁₀ tricyclic carbocycle.

In some embodiments, for the compound or salt of Formula (I), (I-a), (I-b), (I-c), (I-d), (I-e), and (I-f), B is C₆₋₁₀ carbocycle optionally substituted with one or more substituents independently selected from: halogen, —OR¹², N(R¹²)₂, —C(O)R¹², —NO₂, ═O, ═S, ═N(R¹²), —CN, and C₁₋₆ alkyl optionally substituted with one or more substituents independently selected from halogen, —OR¹², N(R¹²)₂, —C(O)R¹², —NO₂, ═O, ═S, ═N(R¹²), and —CN. In some embodiments, B is C₆₋₁₀ carbocycle optionally substituted with one or more substituents independently selected from: halogen, —OR¹², N(R¹²)₂, —C(O)R¹², —NO₂, ═O, ═S, ═N(R¹²), —CN. In some embodiments, B is C₆₋₁₀ carbocycle optionally substituted with C₁₋₆ alkyl optionally substituted with one or more substituents independently selected from halogen, —OR¹², N(R¹²)₂, —C(O)R¹², —NO₂, ═O, ═S, ═N(R¹²), and —CN.

In some embodiments, for the compound or salt of Formula (I), (I-a), (I-b), (I-c), (I-d), (I-e), and (I-f), B is cyclohexyl, cycloheptyl, dispiro[2.0.2⁴.1³]heptane, spiro[3.3]heptane, and indane, any one of which is optionally substituted with one or more substituents independently selected from: halogen, —OR¹², N(R¹²)₂, —NO₂, —CN, and C₁₋₆ alkyl optionally substituted with one or more substituents independently selected from halogen, —OR¹², N(R¹²)₂, —C(O)R¹², —NO₂, ═O, ═S, ═N(R¹²), and —CN. In some embodiments, B is cyclohexyl, cycloheptyl, dispiro[2.0.2⁴.1³]heptane, spiro[3.3]heptane, and indane, any one of which is optionally substituted with one or more substituents independently selected from halogen, C₁₋₆ alkyl, and C₁₋₆ haloalkyl. In some embodiments, B is selected from:

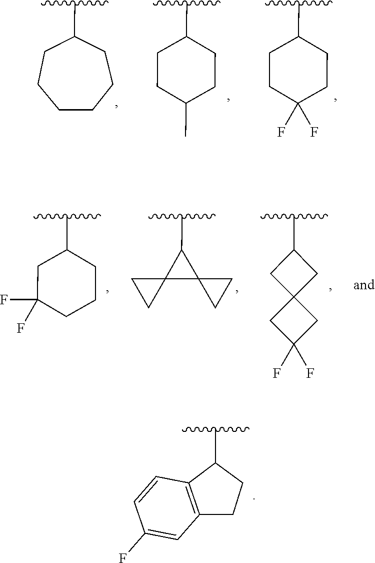

In some embodiments, B is selected from

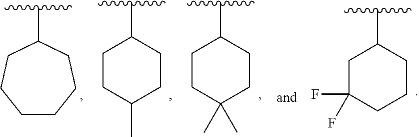

In some embodiments, B is

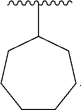 [SMILES: *C1CCCCCC1]

In some embodiments, B is

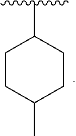 [SMILES: *C1CCC(C)CC1]

In some embodiments, B is

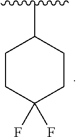 [SMILES: *C1CCC(F)(F)CC1]

In some embodiments, B is

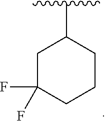 [SMILES: *C1CCCC(F)(F)C1]

In some embodiments, B is selected from

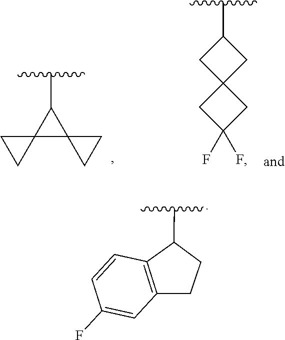

In some embodiments, B is

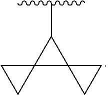 [SMILES: *C1C2(CC2)C12CC2]

In some embodiments, B is

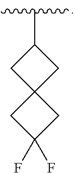 [SMILES: *C1CC2(C1)CC(F)(F)C2]

In some embodiments, B is

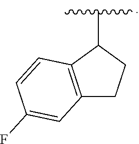 [SMILES: *C1CCc2cc(F)ccc21]

In some embodiments, for the compound or salt of Formula (I), (I-a), (I-b), (I-c), (I-d), (I-e), and (I-f), B is selected from:

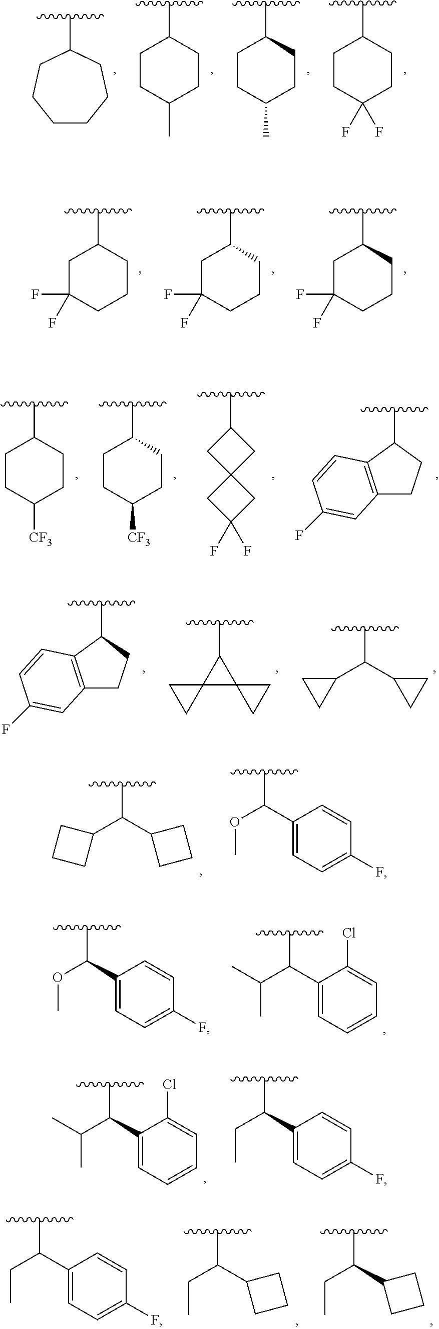

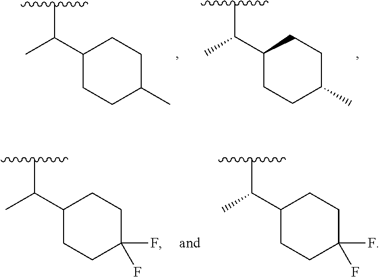

In some embodiments, B is selected from:

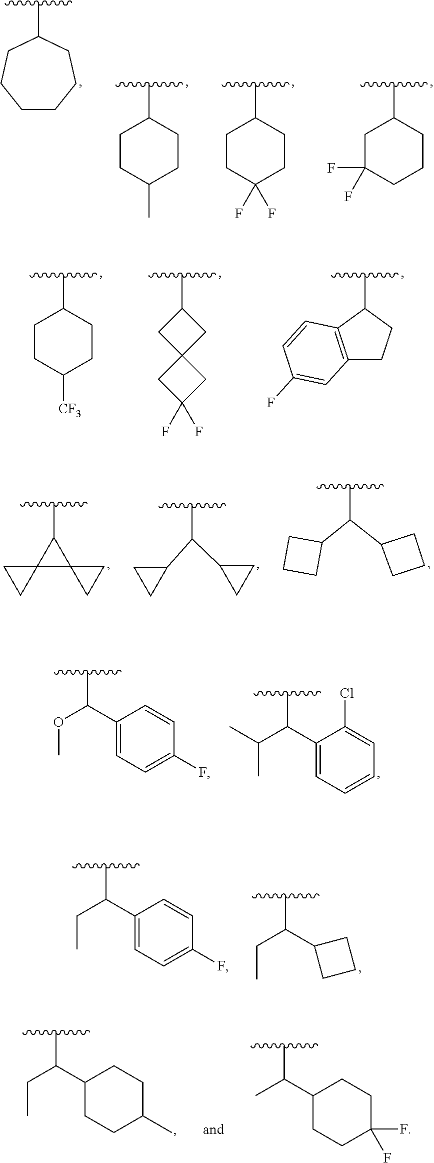

In some embodiments, B is selected from:

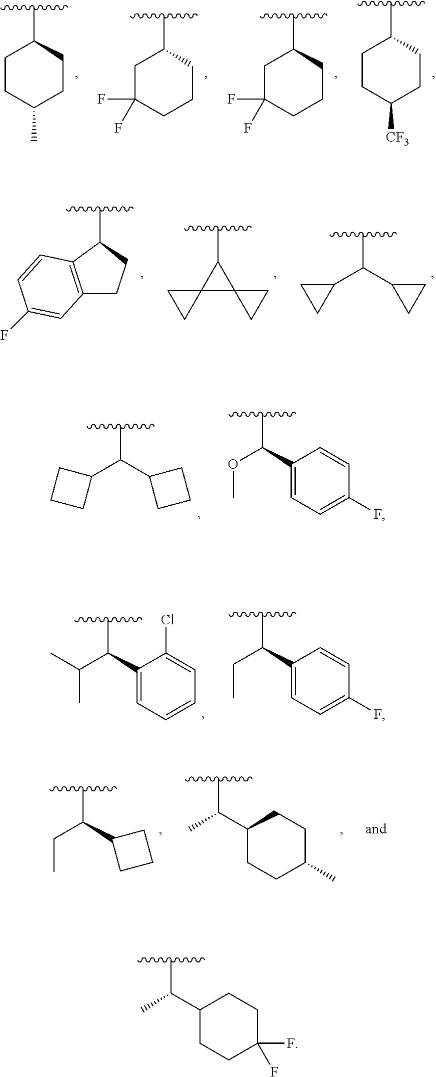

In some embodiments, Formula (I) is selected from:

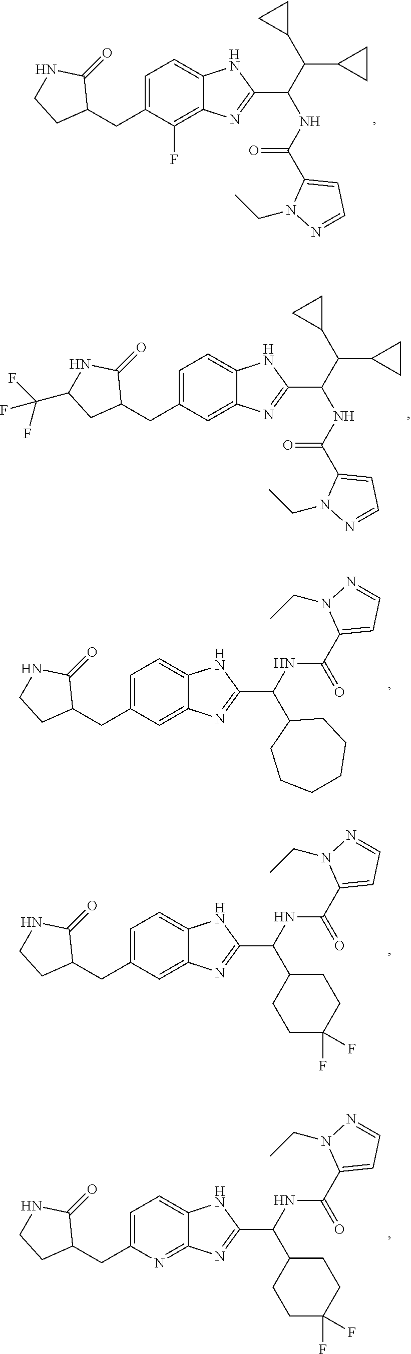 [SMILES: CCn1nccc1C(=O)NC(c1nc2c(F)c(CC3CCNC3=O)ccc2[nH]1)C(C1CC1)C1CC1 ; CCn1nccc1C(=O)NC(c1nc2cc(CC3CC(C(F)(F)F)NC3=O)ccc2[nH]1)C(C1CC1)C1CC1 ; CCn1nccc1C(=O)NC(c1nc2cc(CC3CCNC3=O)ccc2[nH]1)C1CCCCCC1 ; CCn1nccc1C(=O)NC(c1nc2cc(CC3CCNC3=O)ccc2[nH]1)C1CCC(F)(F)CC1 ; CCn1nccc1C(=O)NC(c1nc2nc(CC3CCNC3=O)ccc2[nH]1)C1CCC(F)(F)CC1]

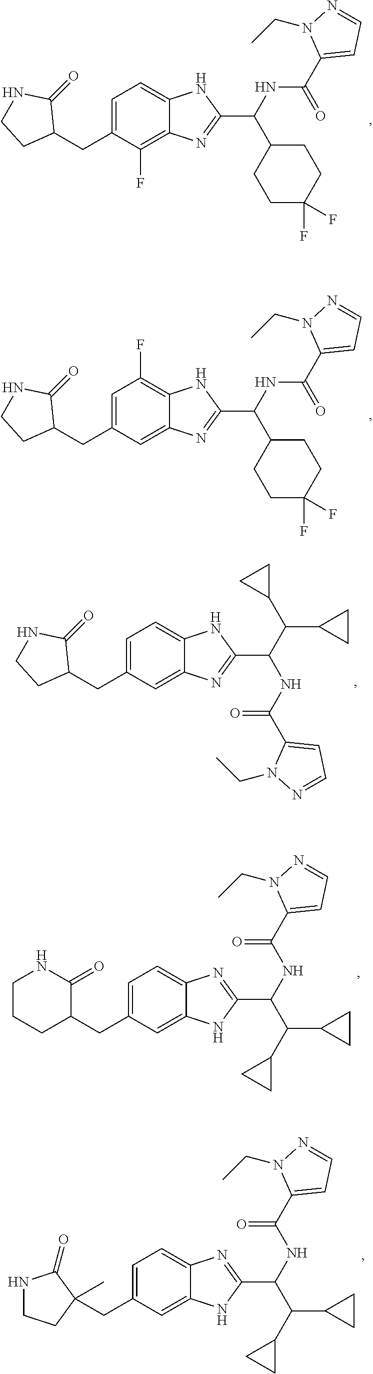 [SMILES: CCn1nccc1C(=O)NC(c1nc2c(F)c(CC3CCNC3=O)ccc2[nH]1)C1CCC(F)(F)CC1 ; CCn1nccc1C(=O)NC(c1nc2cc(CC3CCNC3=O)cc(F)c2[nH]1)C1CCC(F)(F)CC1 ; CCn1nccc1C(=O)NC(c1nc2cc(CC3CCNC3=O)ccc2[nH]1)C(C1CC1)C1CC1 ; CCn1nccc1C(=O)NC(c1nc2ccc(CC3CCCNC3=O)cc2[nH]1)C(C1CC1)C1CC1 ; CCn1nccc1C(=O)NC(c1nc2ccc(CC3(C)CCNC3=O)cc2[nH]1)C(C1CC1)C1CC1]

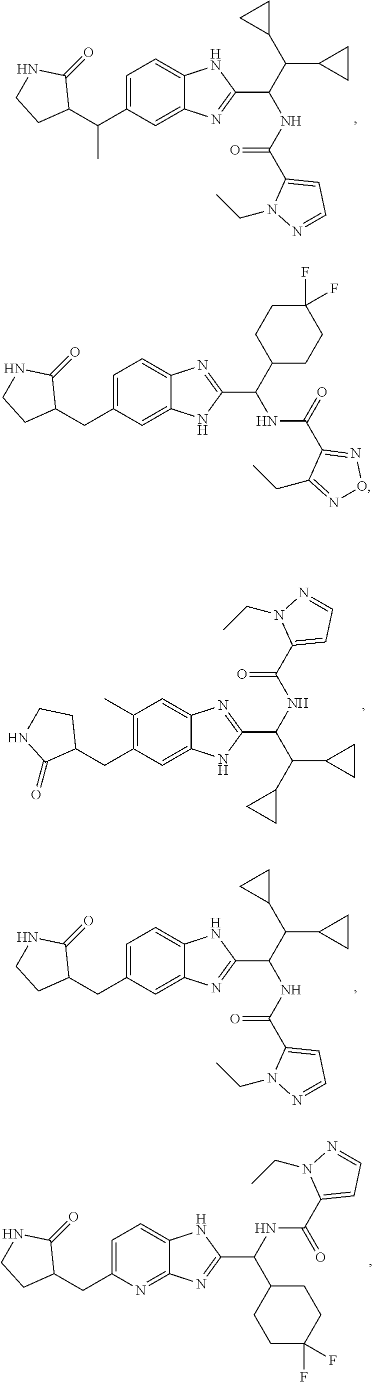

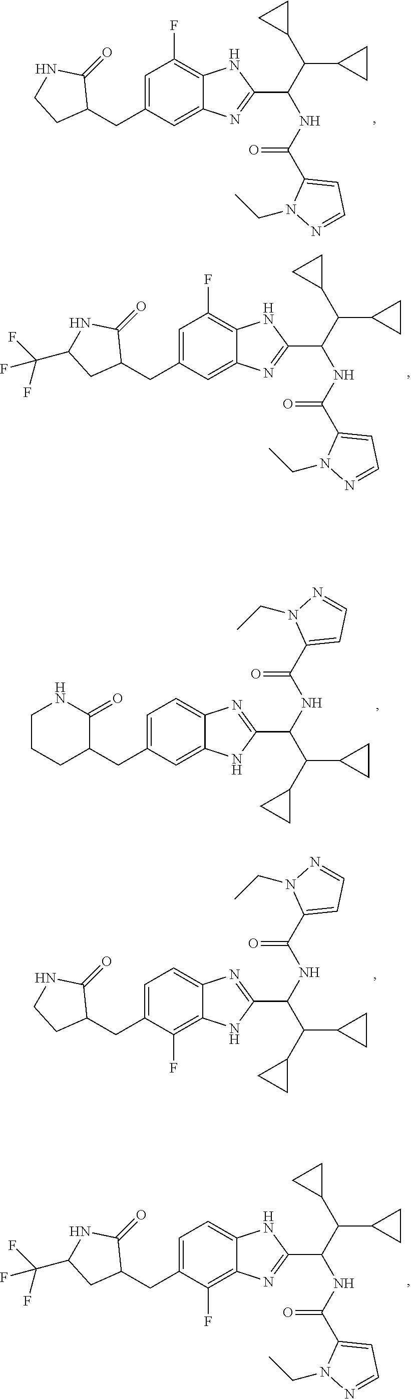

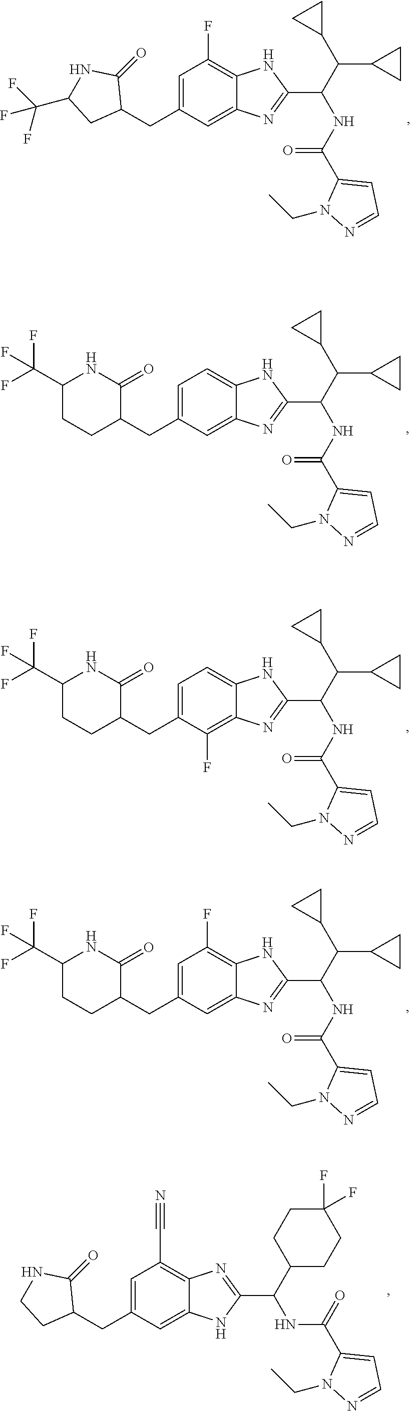 [SMILES: CCn1nccc1C(=O)NC(c1nc2cc(CC3CC(C(F)(F)F)NC3=O)cc(F)c2[nH]1)C(C1CC1)C1CC1 ; CCn1nccc1C(=O)NC(c1nc2cc(CC3CCC(C(F)(F)F)NC3=O)ccc2[nH]1)C(C1CC1)C1CC1 ; CCn1nccc1C(=O)NC(c1nc2c(F)c(CC3CCC(C(F)(F)F)NC3=O)ccc2[nH]1)C(C1CC1)C1CC1 ; CCn1nccc1C(=O)NC(c1nc2cc(CC3CCC(C(F)(F)F)NC3=O)cc(F)c2[nH]1)C(C1CC1)C1CC1 ; CCn1nccc1C(=O)NC(c1nc2c(C#N)cc(CC3CCNC3=O)cc2[nH]1)C1CCC(F)(F)CC1]

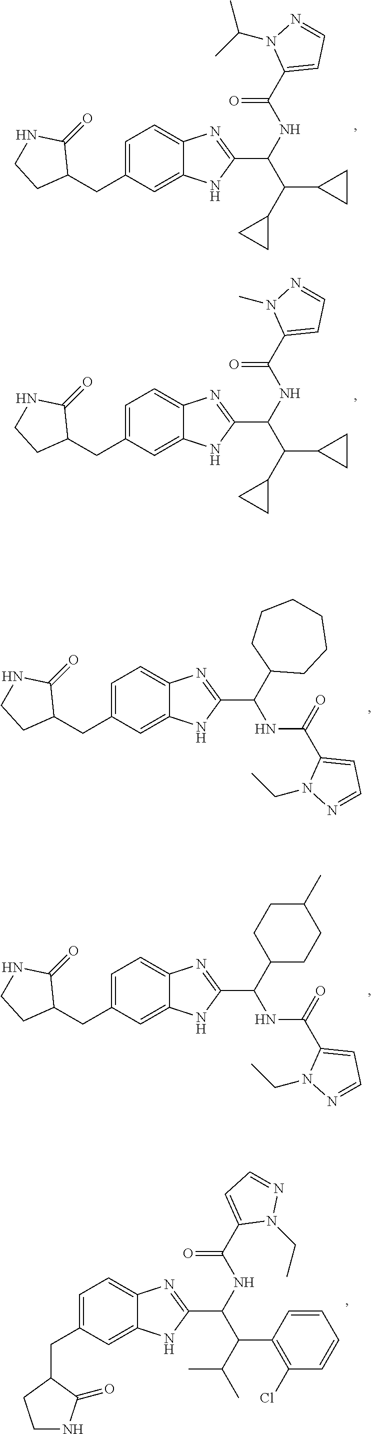

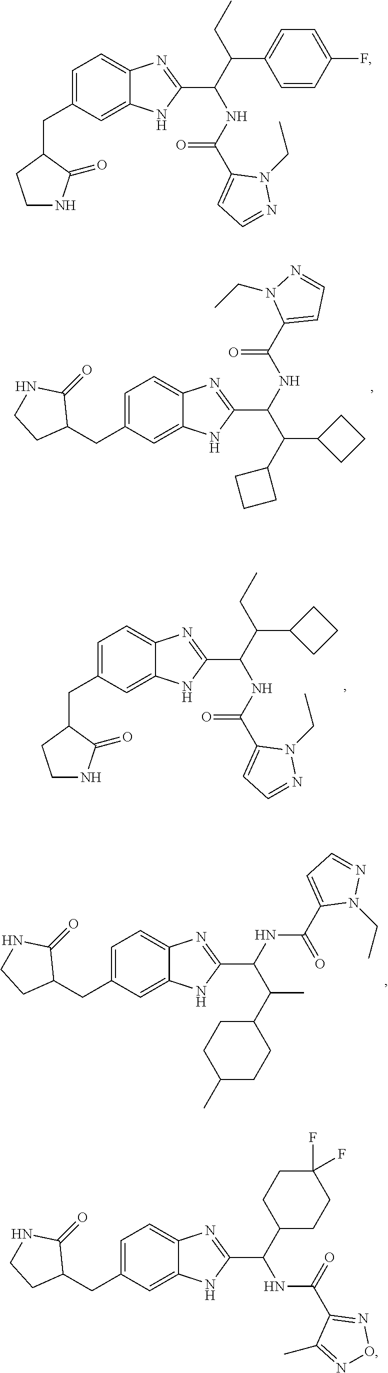 [SMILES: CCC(c1ccc(F)cc1)C(NC(=O)c1ccnn1CC)c1nc2ccc(CC3CCNC3=O)cc2[nH]1 ; CCn1nccc1C(=O)NC(c1nc2ccc(CC3CCNC3=O)cc2[nH]1)C(C1CCC1)C1CCC1 ; CCC(C1CCC1)C(NC(=O)c1ccnn1CC)c1nc2ccc(CC3CCNC3=O)cc2[nH]1 ; CCn1nccc1C(=O)NC(c1nc2ccc(CC3CCNC3=O)cc2[nH]1)C(C)C1CCC(C)CC1 ; Cc1nonc1C(=O)NC(c1nc2ccc(CC3CCNC3=O)cc2[nH]1)C1CCC(F)(F)CC1]

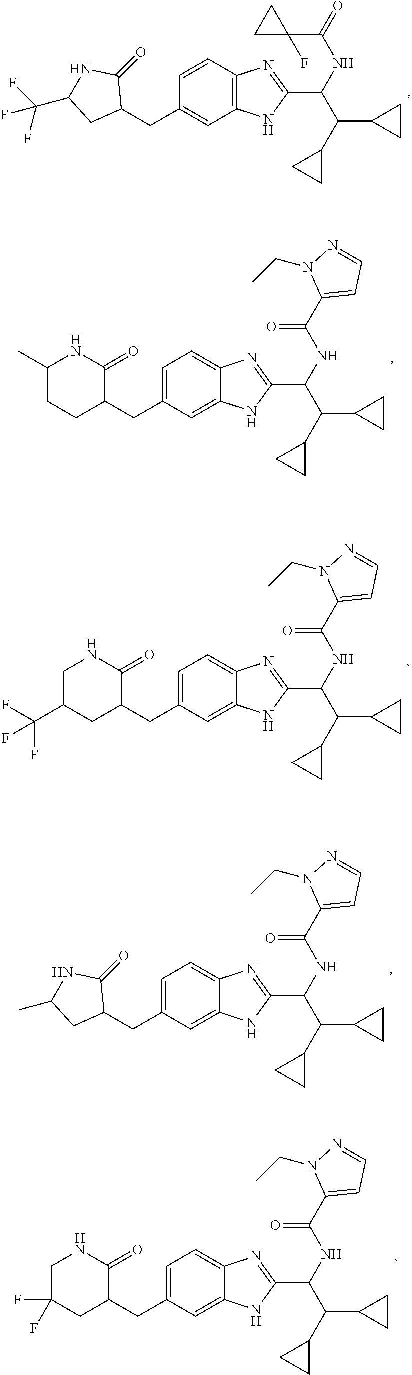 [SMILES: O=C1NC(C(F)(F)F)CC1Cc1ccc2nc(C(NC(=O)C3(F)CC3)C(C3CC3)C3CC3)[nH]c2c1 ; CCn1nccc1C(=O)NC(c1nc2ccc(CC3CCC(C)NC3=O)cc2[nH]1)C(C1CC1)C1CC1 ; CCn1nccc1C(=O)NC(c1nc2ccc(CC3CC(C(F)(F)F)CNC3=O)cc2[nH]1)C(C1CC1)C1CC1 ; CCn1nccc1C(=O)NC(c1nc2ccc(CC3CC(C)NC3=O)cc2[nH]1)C(C1CC1)C1CC1 ; CCn1nccc1C(=O)NC(c1nc2ccc(CC3CC(F)(F)CNC3=O)cc2[nH]1)C(C1CC1)C1CC1]

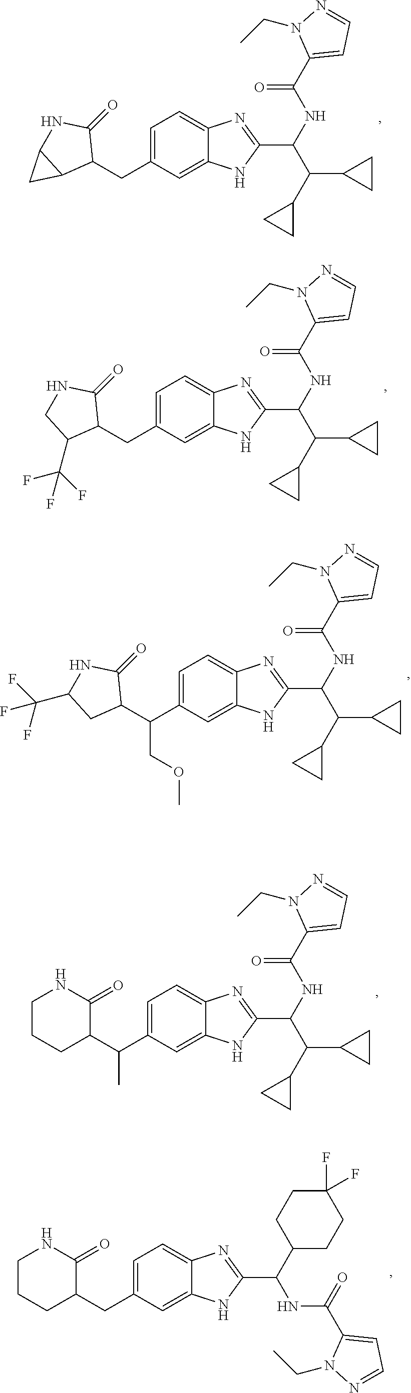 [SMILES: CCn1nccc1C(=O)NC(c1nc2ccc(CC3C(=O)NC4CC43)cc2[nH]1)C(C1CC1)C1CC1 ; CCn1nccc1C(=O)NC(c1nc2ccc(CC3C(=O)NCC3C(F)(F)F)cc2[nH]1)C(C1CC1)C1CC1 ; CCn1nccc1C(=O)NC(c1nc2ccc(C(COC)C3CC(C(F)(F)F)NC3=O)cc2[nH]1)C(C1CC1)C1CC1 ; CCn1nccc1C(=O)NC(c1nc2ccc(C(C)C3CCCNC3=O)cc2[nH]1)C(C1CC1)C1CC1 ; CCn1nccc1C(=O)NC(c1nc2ccc(CC3CCCNC3=O)cc2[nH]1)C1CCC(F)(F)CC1]

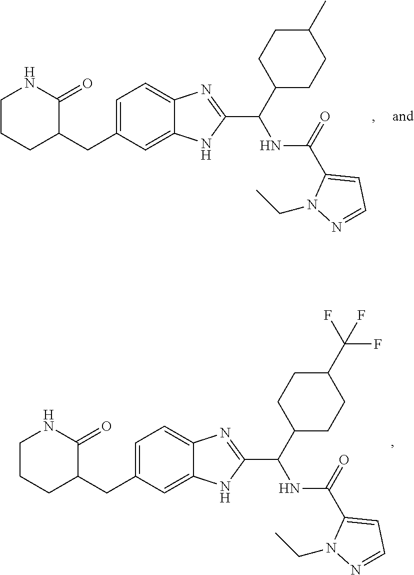 [SMILES: CCn1nccc1C(=O)NC(c1nc2ccc(CC3CCCNC3=O)cc2[nH]1)C1CCC(C)CC1 ; CCn1nccc1C(=O)NC(c1nc2ccc(CC3CCCNC3=O)cc2[nH]1)C1CCC(C(F)(F)F)CC1]

or pharmaceutically acceptable salts thereof. In some embodiments, Formula I is selected from a compound of Table 1, or pharmaceutically acceptable salts thereof.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

Chemical entities having carbon-carbon double bonds or carbon-nitrogen double bonds may exist in Z- or E-form (or cis- or trans-form). Furthermore, some chemical entities may exist in various tautomeric forms. Unless otherwise specified, compounds or salts of Formula (I), (I-a), (I-b), (I-c), (I-d), (I-e), or (I-f), are intended to include all Z-, E- and tautomeric forms as well.

“Isomers” are different compounds that have the same molecular formula. “Stereoisomers” are isomers that differ only in the way the atoms are arranged in space. “Enantiomers” are a pair of stereoisomers that are non-superimposable mirror images of each other. A 1:1 mixture of a pair of enantiomers is a “racemic” mixture. The term “(±)” is used to designate a racemic mixture where appropriate. “Diastereoisomers” or “diastereomers” are stereoisomers that have at least two asymmetric atoms but are not mirror images of each other. The absolute stereochemistry is specified according to the Cahn-Ingold-Prelog R-S system. When a compound is a pure enantiomer, the stereochemistry at each chiral carbon can be specified by either R or S. Resolved compounds whose absolute configuration is unknown can be designated (+) or (−) depending on the direction (dextro- or levorotatory) in which they rotate plane polarized light at the wavelength of the sodium D line. Certain compounds described herein contain one or more asymmetric centers and can thus give rise to enantiomers, diastereomers, and other stereoisomeric forms, the asymmetric centers of which can be defined, in terms of absolute stereochemistry, as (R)- or (S)-. The present chemical entities, pharmaceutical compositions and methods are meant to include all such possible stereoisomers, including racemic mixtures, optically pure forms, mixtures of diastereomers and intermediate mixtures. Optically active (R)- and (S)-isomers can be prepared using chiral synthons or chiral reagents, or resolved using conventional techniques. The optical activity of a compound can be analyzed via any suitable method, including but not limited to chiral chromatography and polarimetry, and the degree of predominance of one stereoisomer over the other isomer can be determined.

The compounds or salts for Formula (I), (I-a), (I-b), (I-c), (I-d), (I-e), or (I-f), may in some cases exist as diastereomers, enantiomers, or other stereoisomeric forms. The compounds presented herein include all diastereomeric, enantiomeric, and epimeric forms as well as the racemates, mixtures of diastereomers, and other mixtures thereof, to the extent they can be made by one of ordinary skill in the art by routine experimentation. Separation of stereoisomers may be performed by chromatography or by forming diastereomers and separating by recrystallization, or chromatography, or any combination thereof. (Jean Jacques, Andre Collet, Samuel H. Wilen, “Enantiomers, Racemates and Resolutions”, John Wiley And Sons, Inc., 1981, herein incorporated by reference for this disclosure). Stereoisomers may also be obtained by stereoselective synthesis. Furthermore, a mixture of two enantiomers enriched in one of the two can be purified to provide further optically enriched form of the major enantiomer by recrystallization and/or trituration.

In certain embodiments, compounds or salts for Formula (I), (I-a), (I-b), (I-c), (I-d), (I-e), or (I-f), may comprise two or more enantiomers or diastereomers of a compound wherein a single enantiomer or diastereomer accounts for at least about 70% by weight, at least about 80% by weight, at least about 90% by weight, at least about 98% by weight, or at least about 99% by weight or more of the total weight of all stereoisomers. Methods of producing substantially pure enantiomers are well known to those of skill in the art. For example, a single stereoisomer, e.g., an enantiomer, substantially free of its stereoisomer may be obtained by resolution of the racemic mixture using a method such as formation of diastereomers using optically active resolving agents (Stereochemistry of Carbon Compounds, (1962) by E. L. Eliel, McGraw Hill; Lochmuller (1975) J. Chromatogr., 113 (3): 283-302). Racemic mixtures of chiral compounds can be separated and isolated by any suitable method, including, but not limited to: (1) formation of ionic, diastereomeric salts with chiral compounds and separation by fractional crystallization or other methods, (2) formation of diastereomeric compounds with chiral derivatizing reagents, separation of the diastereomers, and conversion to the pure stereoisomers, and (3) separation of the substantially pure or enriched stereoisomers directly under chiral conditions. Another approach for separation of the enantiomers is to use a Diacel chiral column and elution using an organic mobile phase such as done by Chiral Technologies (www.chiraltech.com) on a fee for service basis.

A “tautomer” refers to a molecule wherein a proton shift from one atom of a molecule to another atom of the same molecule is possible. In certain embodiments, the compounds or salts for Formula (I), (I-a), (I-b), (I-c), (I-d), (I-e), or (I-f), exist as tautomers. In circumstances where tautomerization is possible, a chemical equilibrium of the tautomers may exist. The exact ratio of the tautomers depends on several factors, including physical state, temperature, solvent, and pH. Some non-limiting examples of tautomeric equilibrium include:

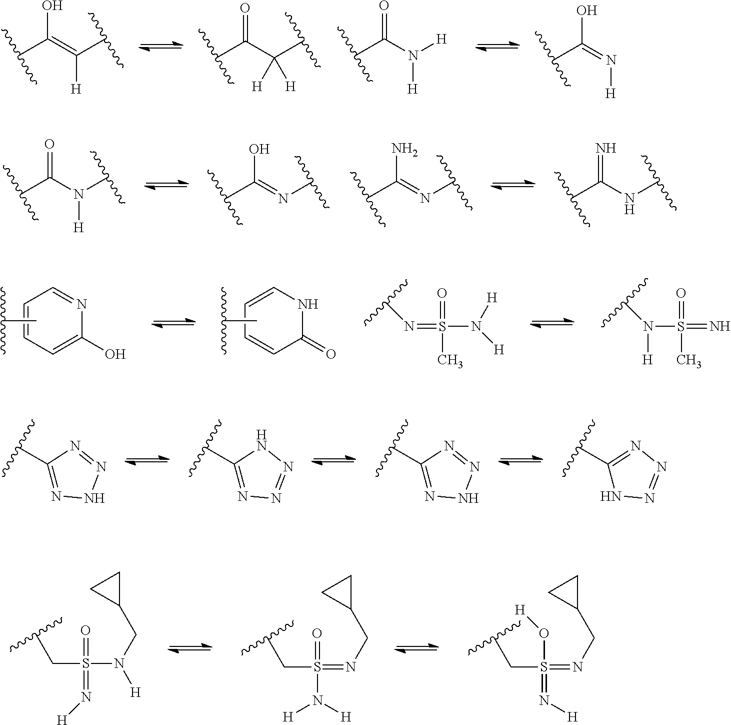

The compounds of Formula (I), (I-a), (I-b), (I-c), (I-d), (I-e), or (I-f), can be used in different enriched isotopic forms, e.g., enriched in the content of ²H, ³H, ¹¹C, ¹³C and/or ¹⁴C. In one particular embodiment, the compound is deuterated in at least one position. Such deuterated forms can be made by the procedure described in U.S. Pat. Nos. 5,846,514 and 6,334,997. As described in U.S. Pat. Nos. 5,846,514 and 6,334,997, deuteration can improve the metabolic stability and or efficacy, thus increasing the duration of action of drugs.

In certain embodiments, the compounds of Formula (I), (I-a), (I-b), (I-c), (I-d), (I-e), or (I-f), have some or all of the ¹H atoms replaced with ²H atoms. The methods of synthesis for deuterium-containing compounds are known in the art and include, by way of non-limiting example only, the following synthetic methods.

Deuterium substituted compounds are synthesized using various methods such as described in: Dean, Dennis C.; Editor. Recent Advances in the Synthesis and Applications of Radiolabeled Compounds for Drug Discovery and Development. [In: Curr., Pharm. Des., 2000; 6 (10)] 2000, 110 pp; George W.; Varma, Rajender S. The Synthesis of Radiolabeled Compounds via Organometallic Intermediates, Tetrahedron, 1989, 45 (21), 6601-21; and Evans, E. Anthony. Synthesis of radiolabeled compounds, J. Radioanal. Chem., 1981, 64 (1-2), 9-32.

Deuterated starting materials are readily available and are subjected to the synthetic methods described herein to provide for the synthesis of deuterium-containing compounds. Large numbers of deuterium-containing reagents and building blocks are available commercially from chemical vendors, such as Aldrich Chemical Co.

Unless otherwise stated, compounds of Formula (I), (I-a), (I-b), (I-c), (I-d), (I-e), or (I-f), are intended to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of the present disclosure.

The compounds of Formula (I), (I-a), (I-b), (I-c), (I-d), (I-e), or (I-f), optionally contain unnatural proportions of atomic isotopes at one or more atoms that constitute such compounds. For example, the compounds may be labeled with isotopes, such as for example, deuterium (²H), tritium (³H), iodine-125 (¹²⁵I) or carbon-14 (¹⁴C). Isotopic substitution with ²H, ¹¹C, ¹³C, ¹⁴C, ¹⁵C, ¹²N, ¹³N, ¹⁵N, ¹⁶N, ¹⁶O, ¹⁷O, ¹⁴F, ¹⁵F, ¹⁶F, ¹⁷F, ¹⁸F, ³³S, ³⁴S, ³⁵S, ³⁶S, ³⁵Cl, ³⁷Cl, ⁷⁹Br, ⁸¹Br, and ¹²⁵I are all contemplated. All isotopic variations of the compounds of the present invention, whether radioactive or not, are encompassed within the scope of the present invention.

Included in the present disclosure are salts, particularly pharmaceutically acceptable salts, of the compounds of Formula (I), (I-a), (I-b), (I-c), (I-d), (I-e), or (I-f). The compounds of the present disclosure may possess a sufficiently acidic, a sufficiently basic, or both functional groups, can react with any of a number of inorganic bases, and inorganic and organic acids, to form a salt. Alternatively, compounds that are inherently charged, such as those with a quaternary nitrogen, can form a salt with an appropriate counterion, e.g., a halide such as bromide, chloride, or fluoride.

In certain embodiments, compounds or salts of Formula (I), (I-a), (I-b), (I-c), (I-d), (I-e), or (I-f), may be prodrugs. The term “prodrug” is intended to encompass compounds which, under physiologic conditions, are converted into pharmaceutical agents of the present disclosure. One method for making a prodrug is to include one or more selected moieties which are hydrolyzed under physiologic conditions to reveal the desired molecule. In other embodiments, the prodrug is converted by an enzymatic activity of the host animal such as specific target cells in the host animal.

In some embodiments, the design of a prodrug increases the lipophilicity of the pharmaceutical agent. In some embodiments, the design of a prodrug increases the effective water solubility. See, e.g., Fedorak et al., Am. J. Physiol., 269: G210-218 (1995); McLoed et al., Gastroenterol, 106:405-413 (1994); Hochhaus et al., Biomed. Chrom., 6:283-286 (1992); J. Larsen and H. Bundgaard, Int. J. Pharmaceutics, 37, 87 (1987); J. Larsen et al., Int. J. Pharmaceutics, 47, 103 (1988); Sinkula et al., J. Pharm. Sci., 64:181-210 (1975); T. Higuchi and V. Stella, Pro-drugs as Novel Delivery Systems, Vol. 14 of the A.C.S. Symposium Series; and Edward B. Roche, Bioreversible Carriers in Drug Design, American Pharmaceutical Association and Pergamon Press, 1987, all incorporated herein for such disclosure). According to another embodiment, the present disclosure provides methods of producing the above-defined compounds. The compounds may be synthesized using conventional techniques. Advantageously, these compounds are conveniently synthesized from readily available starting materials.

Synthetic chemistry transformations and methodologies useful in synthesizing the compounds described herein are known in the art and include, for example, those described in R. Larock, Comprehensive Organic Transformations (1989); T. W. Greene and P. G. M. Wuts, Protective Groups in Organic Synthesis, 2d. Ed. (1991); L. Fieser and M. Fieser, Fieser and Fieser's Reagents for Organic Synthesis (1994); and L. Paquette, ed., Encyclopedia of Reagents for Organic Synthesis (1995).

### Pharmaceutical Formulations

In some aspects, the present disclosure provides a pharmaceutical composition comprising a compound or salt of Formula (I), (I-a), (I-b), (I-c), (I-d), (I-e), or (I-f), and at least one pharmaceutically acceptable excipient. In some embodiments, the pharmaceutical composition comprises a compound or salt of Formula (I), (I-a), (I-b), (I-c), (I-d), (I-e), or (I-f), and a pharmaceutically acceptable excipient.

Pharmaceutical compositions can be formulated using one or more physiologically-acceptable carriers comprising excipients and auxiliaries. Formulation can be modified depending upon the route of administration chosen. Pharmaceutical compositions comprising a compound, salt or conjugate can be manufactured, for example, by lyophilizing the compound, salt or conjugate, mixing, dissolving, emulsifying, encapsulating or entrapping the conjugate. The pharmaceutical compositions can also include the compounds, salts or conjugates in a free-base form or pharmaceutically-acceptable salt form.

Pharmaceutical compositions as often further can comprise more than one active compound (e.g., a compound, salt or conjugate and other agents) as necessary for the particular indication being treated. The active compounds can have complementary activities that do not adversely affect each other. Such molecules can be present in combination in amounts that are effective for the purpose intended.

A compound or salt of any one of Formula (I), (I-a), (I-b), (I-c), (I-d), (I-e), or (I-f), may be formulated in any suitable pharmaceutical formulation. A pharmaceutical formulation of the present disclosure typically contains an active ingredient (e.g., compound or salt of any one Formula I) and one or more pharmaceutically acceptable excipients or carriers, including but not limited to: inert solid diluents and fillers, diluents, sterile aqueous solution and various organic solvents, permeation enhancers, antioxidants, solubilizers, and adjuvants.

In certain embodiments, a compound or salt of Formula (I), (I-a), (I-b), (I-c), (I-d), (I-e), or (I-f), is formulated with a chelating agent or other material capable of binding metal ions, such as ethylene diamine tetra acetic acid (EDTA) and its salts are capable of enhancing the stability of a compound or salt of Formula (I), (I-a), (I-b), (I-c), (I-d), (I-e), or (I-f).

Pharmaceutical formulations may be provided in any suitable form, which may depend on the route of administration.

In some embodiments, the disclosure provides a pharmaceutical composition for oral administration containing at least one compound or salt of any one of Formula (I), (I-a), (I-b), (I-c), (I-d), (I-e), or (I-f), and a pharmaceutical excipient suitable for oral administration. The composition may be in the form of a solid, liquid, gel, semi-liquid, or semi-solid. In some embodiments, the composition further comprises a second agent.

Pharmaceutical compositions of the disclosure suitable for oral administration can be presented as discrete dosage forms, such as hard or soft capsules, cachets, troches, lozenges, or tablets, or liquids or aerosol sprays each containing a predetermined amount of an active ingredient as a powder or in granules, a solution, or a suspension in an aqueous or non-aqueous liquid, an oil-in-water emulsion, or a water-in-oil liquid emulsion, or dispersible powders or granules, or syrups or elixirs. Such dosage forms can be prepared by any of the methods of pharmacy, which typically include the step of bringing the active ingredient(s) into association with the carrier. In general, the composition are prepared by uniformly and intimately admixing the active ingredient(s) with liquid carriers or finely divided solid carriers or both, and then, if necessary, shaping the product into the desired presentation. For example, a tablet can be prepared by compression or molding, optionally with one or more accessory ingredients. Compressed tablets can be prepared by compressing in a suitable machine the active ingredient(s) in a free-flowing form such as powder or granules, optionally mixed with an excipient such as, but not limited to, a binder, a lubricant, an inert diluent, and/or a surface active or dispersing agent. Molded tablets can be made by molding in a suitable machine a mixture of the powdered compound or salt of any one of Formula (I), (I-a), (I-b), (I-c), (I-d), (I-e), or (I-f), moistened with an inert liquid diluent.

Pharmaceutical compositions may also be prepared from a compound or salt of any one of Formula (I), (I-a), (I-b), (I-c), (I-d), (I-e), or (I-f), and one or more pharmaceutically acceptable excipients. Preparations for such pharmaceutical composition are well-known in the art. See, e.g., Anderson, Philip O.; Knoben, James E.; Troutman, William G, eds., Handbook of Clinical Drug Data, Tenth Edition, McGraw-Hill, 2002; Pratt and Taylor, eds., Principles of Drug Action, Third Edition, Churchill Livingston, New York, 1990; Katzung, ed., Basic and Clinical Pharmacology, Ninth Edition, McGraw Hill, 2003; Goodman and Gilman, eds., The Pharmacological Basis of Therapeutics, Tenth Edition, McGraw Hill, 2001; Remingtons Pharmaceutical Sciences, 20th Ed., Lippincott Williams & Wilkins., 2000; Martindale, The Extra Pharmacopoeia, Thirty-Second Edition (The Pharmaceutical Press, London, 1999).

### Methods of Treatment

In some aspects, the present disclosure provides a method of modulating IL-17 A in a subject in need thereof, comprising administering to the subject a compound or salt of Formula (I), (I-a), (I-b), (I-c), (I-d), (I-e), or (I-f), or a pharmaceutical composition thereof.

Increased levels of IL-17A have been associated with several conditions including airway inflammation, rheumatoid arthritis (RA), osteoarthritis, bone erosion, intraperitoneal abscesses and adhesions, inflammatory bowel disorder (IBD), allograft rejection, psoriasis, psoriatic arthritis, ankylosing spondylitis, certain types of cancer, angiogenesis, atherosclerosis and multiple sclerosis (MS). Both IL-17A and IL-17R are upregulated in the synovial tissue of RA patients. IL-17A exerts its role in pathogenesis of RA through IL-1-β and TNF-α dependent and independent pathways. IL-17A stimulates secretion of other cytokines and chemokines, e.g., TNF-α, IL-1B, IL-6, IL-8 and Gro-α. IL-17A directly contributes to disease progression in RA. Injection of IL-17A into the mouse knee promotes joint destruction independently of IL-I β activity (Ann Rheum Dis 2000, 59:529-32). Anti-IL-1B antibody has no effect on IL-17A induced inflammation and joint damage (J. Immunol 2001, 167:1004-1013). In a streptococcal cell wall (SCW)-induced murine arthritis model, IL-17A induced inflammatory cell infiltration and proteoglycan depletion in wild-type and IL-1β knockout and TNF-α knockout mice. IL-17A knockout mice are phenotypically normal in the absence of antigenic challenge but have markedly reduced arthritis following type II collagen immunization (J. Immunol 2003, 171:6173-6177). Increased levels of IL-17A-secreting cells have also been observed in the facet joints of patients suffering from ankylosing spondylitis (H Appel et al., Arthritis Res Therap. 2011, 13: R95).

Multiple sclerosis is an autoimmune disease characterized by central nervous system (CNS) inflammation with damage to the myelin sheath surrounding axons. A hallmark of MS is that T cells infiltrate into the CNS. Higher numbers of IL-17A mRNA-expressing blood mono-nuclear cells (MNC) are detected during MS clinical exacerbation compared to remission (Multiple Sclerosis, 5:101-104, 1999). Furthermore, experimental autoimmune encephalomyelitis (“EAE”), a preclinical animal model for MS is significantly suppressed in IL-17A knockout mice.

In certain aspects, the disclosure provides methods of modulating IL-17A in a subject in need thereof, comprising administering to said subject a compound or salt of Formula (I), (I-a), (I-b), (I-c), (I-d), (I-e), or (I-f). In certain embodiments, a compound or salt of Formula (I), (I-a), (I-b), (I-c), (I-d), (I-e), or (I-f), inhibits the activity of IL-17A in a subject in need thereof.

In certain embodiments, a compound or salt of Formula (I), (I-a), (I-b), (I-c), (I-d), (I-e), or (I-f), can be used to treat or prevent a disease or condition that is mediated directly or indirectly by IL-17A. Such diseases include inflammatory diseases and conditions, proliferative diseases (e.g., cancer), autoimmune diseases and other disease described herein. The methods generally involve administering therapeutically effective amounts of compounds disclosed herein or a pharmaceutical composition thereof to the subject. In some embodiments, the inflammatory disease or condition is selected from plaque psoriasis, guttate psoriasis, inverse psoriasis, pustular psoriasis, erythrodermic psoriasis, psoriatic arthritis, ankylosing spondylitis, hidradenitis suppurativa, rheumatoid arthritis, palmoplantar psoriasis, spondyloarthritis, and Non-infectious Uveitis.

In some aspects, the present disclosure provides a method of a method of treating or preventing an inflammatory disease or condition in a subject in need thereof, comprising administering to the subject a compound or salt of Formula (I), (I-a), (I-b), (I-c), (I-d), (I-e), or (I-f), or a pharmaceutical composition thereof. In certain embodiments, a compound or salt of Formula (I), (I-a), (I-b), (I-c), (I-d), (I-e), or (I-f), is administered to a subject in need thereof to treat an inflammatory disease or condition, e.g., psoriasis.

In certain embodiments, a compound or salt of Formula (I), (I-a), (I-b), (I-c), (I-d), (I-e), or (I-f), is used to treat or prevent an inflammatory disease or condition is selected from, plaque psoriasis, guttate psoriasis, inverse psoriasis, pustular psoriasis, erythrodermic psoriasis, psoriatic arthritis, ankylosing spondylitis, hidradenitis suppurativa, rheumatoid arthritis, Palmoplantar Psoriasis, Spondyloarthritis, and Non-infectious Uveitis. In certain embodiments, a compound or salt of Formula (I), (I-a), (I-b), (I-c), (I-d), (I-e), or (I-f), is used to treat or prevent psoriasis. In certain embodiments, a compound or salt of Formula (I), (I-a), (I-b), (I-c), (I-d), (I-e), or (I-f), is used for the treatment or prevention of a condition including, but not limited to, airway inflammation, ankylosing spondylitis, asthma, RA (including juvenile RA), as well as other inflammatory disorders, conditions, or diseases.

### EXAMPLES

The invention now being generally described, it will be more readily understood by reference to the following examples which are included merely for purposes of illustration of certain aspects and embodiments of the present invention, and are not intended to limit the invention in any way.

The following synthetic schemes are provided for purposes of illustration, not limitation. The following examples illustrate the various methods of making compounds described herein. It is understood that one skilled in the art may be able to make these compounds by similar methods or by combining other methods known to one skilled in the art. It is also understood that one skilled in the art would be able to make, in a similar manner as described below by using the appropriate starting materials and modifying the synthetic route as needed. In general, starting materials and reagents can be obtained from commercial vendors or synthesized according to sources known to those skilled in the art or prepared as described herein.

Examples 1-2 show general and exemplary procedures for the preparation of the claimed IL-17A modulators. Table 1 demonstrates the exemplary IL-17A modulators synthetized using analogous methods to those described in Examples 1-2. Example 3 provides IL-17 A/A bioassay inhibition data.

### Example 1: Exemplary Synthesis of Compounds 1-2

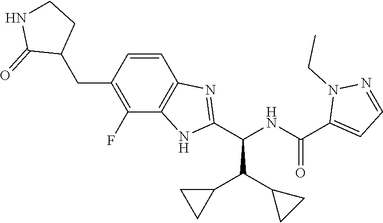 [SMILES: CCn1nccc1C(=O)N[C@H](c1nc2ccc(CC3CCNC3=O)c(F)c2[nH]1)C(C1CC1)C1CC1]

### N-((1S)-2,2-dicyclopropyl-1-(7-fluoro-6-((2-oxopyrrolidin-3-yl)methyl)-1H-benzo[d]imidazol-2-yl)ethyl)-1-ethyl-1H-pyrazole-5-carboxamide. (1-2)

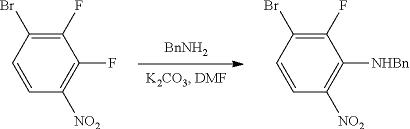

Step 1: To a solution of 1-bromo-2,3-difluoro-4-nitrobenzene (10.0 g, 42.0 mmol, 1.00 eq) and phenylmethanamine (4.95 g, 46.2 mmol, 5.04 mL, 1.10 eq) in DMF (50.0 mL) was added K₂CO₃ (17.4 g, 126 mmol, 3.00 eq). The mixture was stirred at RT for 2 h. The reaction mixture was diluted with water and extracted with EtOAc. The combined organic layers were washed with brine, dried over Na₂SO₄, filtered, and concentrated under reduced pressure to afford N-benzyl-3-bromo-2-fluoror-6-nitroaniline (13.0 g, 95% yield) as a yellow solid. H NMR (400 MHZ, CDCl₃) δ 8.21 (s, 1H), 7.88 (dd, J₁=2.0 Hz, J₂=9.6 Hz, 1H), 7.40-7.32 (m, 5H), 6.85 (dd, J₁=6.0 Hz, J₂=9.2 Hz, 1H), 4.82-4.70 (t, J=4.8 Hz, 2H).

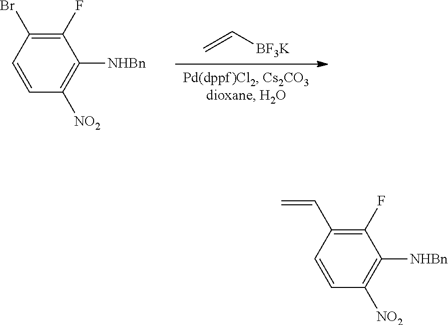

Step 2: To a solution of N-benzyl-3-bromo-2-fluoro-6-nitroaniline (10.0 g, 30.7 mmol, 1.00 eq) and potassium vinyltrifluoroborate (12.3 g, 92.2 mmol, 3.00 eq) in dioxane (100 mL) and H₂O (10.0 mL) was added Cs₂CO₃ (20.0 g, 61.5 mmol, 2.00 eq) and Pd(PPh₃)₄ (3.55 g, 3.08 mmol, 0.100 eq). The mixture was stirred at 100° C. for 8 h. The reaction mixture was diluted with H₂O and extracted with EtOAc. The combined organic layers were dried over Na₂SO₄, filtered, and concentrated under reduced pressure to give a residue. The residue was purified by column chromatography (SiO₂, petroleum ether:ethyl acetate) to afford N-benzyl-2-fluoro-6-nitro-3-vinylaniline (8.00 g, 29.3 mmol, 95% yield) as a white solid. H NMR (400 MHZ, CDCl₃) δ 8.23 (s, 1H), 7.94 (d, J=8.8 Hz, 1H), 7.37-7.30 (m, 5H), 6.89-6.79 (m, 2H), 5.92 (d, J=18.0 Hz, 1H), 5.56 (d, J=10.8 Hz, 1H), 4.78 (t, J=4.4 Hz, 2H).

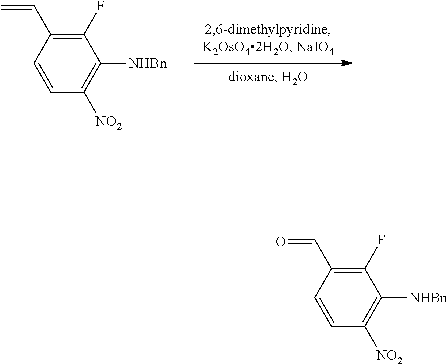

Step 3: To a solution of N-benzyl-2-fluoro-6-nitro-3-vinylaniline (5.00 g, 18.3 mmol, 1.00 eq) and 2,6-dimethylpyridine (3.94 g, 36.7 mmol, 4.28 mL, 2.00 eq) in dioxane (50.0 mL) and H₂O (10.0 mL) was added K₂OsO₄·2H₂O (676 mg, 1.84 mmol, 0.100 eq) at 0° C., and the mixture was stirred at RT for 1 h. Then, NaIO₄ (15.7 g, 73.4 mmol, 4.07 mL, 4.00 eq) was added at 0° C. The mixture was stirred RT for 1 h. The reaction mixture was treated with sat. aq. Na₂S₂O₃ (20.0 mL) at 0° C., and then diluted with H₂O (30.0 mL) and extracted with EtOAc. The combined organic layers were dried over Na₂SO₄, filtered, and concentrated under reduced pressure to give a residue. The residue was purified by column chromatography (SiO₂, petroleum ether:ethyl acetate) to afford 3-(benzylamino)-2-fluoro-4-nitrobenzaldehyde (3.00 g, 10.9 mmol, 59% yield) as a yellow solid. H NMR (400 MHZ, CDCl₃) δ 8.15 (s, 1H), 8.04 (d, J=8.8 Hz, 1H), 7.39-7.33 (m, 5H), 7.11 (dd, J₁=6.0 Hz, J₂=9.2 Hz, 1H), 4.82 (t, J=4.2 Hz, 2H).

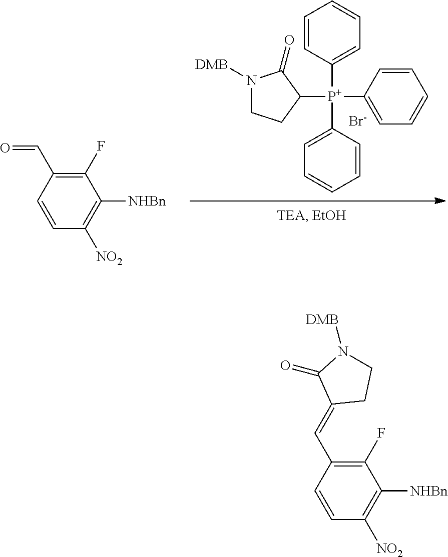

Step 4: To a solution of 3-(benzylamino)-2-fluoro-4-nitrobenzaldehyde (2.00 g, 7.29 mmol, 1.00 eq) and (1-(3,4-dimethylbenzyl)-2-oxopyrrolidin-3-yl)triphenylphosphonium bromide (5.04 g, 8.75 mmol, 1.20 eq) in EtOH (20.0 mL) was added TEA (2.21 g, 21.8 mmol, 3.05 mL, 3.00 eq). The mixture was stirred at 70° C. for 2 h. The reaction mixture was diluted with H₂O and extracted with EtOAc. The combined organic layers were washed with sat. aq. NaHCO₃, dried over Na₂SO₄, filtered, and concentrated under reduced pressure to give a residue. The residue was purified by column chromatography (SiO₂, petroleum ether:ethyl acetate=100:1 to 10:1) to afford (E)-3-(3-(benzylamino)-2-fluoro-4-nitrobenzylidene)-1-(3,4-dimethylbenzyl) pyrrolidin-2-one (2.00 g, 4.07 mmol, 55.8% yield) as a yellow solid. H NMR 400 MHZ, CDCl₃) § 8.22-8.20 (m, 1H), 7.95 (dd, J₁=9.2 Hz, J₂=10.4 Hz, 1H), 7.45 (s, 1H), 7.35-7.25 (m, 5H), 7.17 (dd, J₁=8.8 Hz, J₂=10.0 Hz, 1H), 6.75-6.71 (m, 1H), 6.47-6.45 (m, 2H), 4.78-4.75 (m, 2H), 4.59 (d, J=3.6 Hz, 2H), 3.82-3.74 (m, 6H), 3.39 (t, J=6.0 Hz, 2H), 2.90-2.87 (m, 2H).

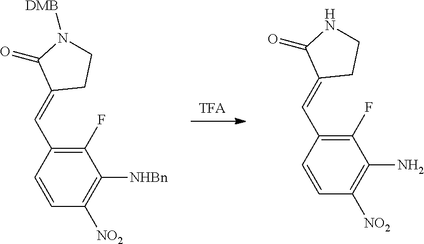

Step 5: (E)-3-(3-(Benzylamino)-2-fluoro-4-nitrobenzylidene)-1-(3,4-dimethylbenzyl) pyrrolidin-2-one (1.50 g, 3.05 mmol, 1.00 eq) was diluted with 3.0 mL of TFA in a microwave tube. The sealed tube was heated at 110° C. for 1 h under microwave. The reaction mixture was diluted with H₂O and adjusted to pH 9 by adding sat. aq. NaHCO₃. The mixture was extracted with DCM. The combined organic layers were dried over Na₂SO₄, filtered, and concentrated under reduced pressure to give a residue. The crude product was triturated with MTBE (30.0 mL) at RT for 30 min and filtered to afford (E)-3-(3-amino-2-fluoro-4-nitrobenzylidene) pyrrolidin-2-one (700 mg, 2.79 mmol, 91.3% yield) as a yellow solid. LCMS [M+H]⁺=252.0 m/z.

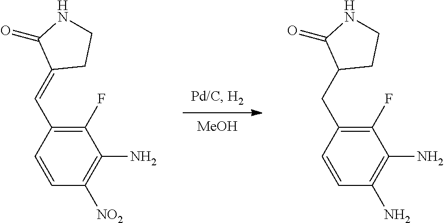 [SMILES: "MeOH" ; Nc1c([N+](=O)[O-])ccc(/C=C2\CCNC2=O)c1F ; "Pd/C, H2" ; Nc1ccc(CC2CCNC2=O)c(F)c1N]

Step 6: To a solution of (E)-3-(3-amino-2-fluoro-4-nitrobenzylidene) pyrrolidin-2-one (700 mg, 2.79 mmol, 1.00 eq) in MeOH (5.00 mL) was added Pd/C (70.0 mg, 10.0% purity) under N₂. The suspension was degassed and purged with H₂ 3 times. The mixture was stirred under H₂ (50 Psi) at RT for 12 h. The reaction mixture was filtered, and the filter cake was washed with 100 mL of MeOH. The filtrate was concentrated under reduced pressure to give a residue. The residue was purified by prep-TLC (SiO₂, ethyl acetate:methanol=10:1) to afford 3-(3,4-diamino-2-fluorobenzyl) pyrrolidin-2-one (240 mg, 1.08 mmol, 38.5% yield) as a yellow solid.

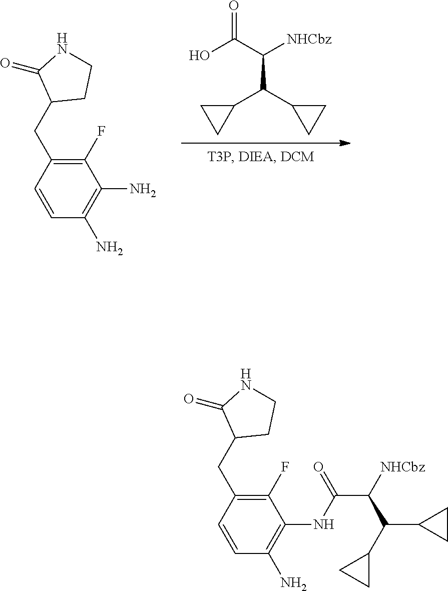 [SMILES: Nc1ccc(CC2CCNC2=O)c(F)c1N ; O=C(N[C@H](C(=O)O)C(C1CC1)C1CC1)OCc1ccccc1 ; "T3P, DIEA, DCM" ; Nc1ccc(CC2CCNC2=O)c(F)c1NC(=O)[C@@H](NC(=O)OCc1ccccc1)C(C1CC1)C1CC1]

Step 7: To a solution of 3-(3,4-diamino-2-fluorobenzyl) pyrrolidin-2-one (110 mg, 492 μmol, 1.00 eq) and(S)-2-(((benzyloxy) carbonyl)amino)-3,3-dicyclopropylpropanoic acid (74.7 mg, 246 μmol, 0.500 eq) in DCM (5.00 mL) was added T3P (940 mg, 1.48 mmol, 879 μL, 50.0% purity, 3.00 eq) and DIEA (318 mg, 2.46 mmol, 429 μL, 5.00 eq) at 0° C. The mixture was stirred at RT for 2 h. The reaction mixture was diluted with water and extracted with EtOAc. The combined organic layers were washed with sat. aq. NaHCO₃, dried over Na₂SO₄, filtered, and concentrated under reduced pressure to afford benzyl ((2S)-1-((6-amino-2-fluoro-3-((2-oxopyrrolidin-3-yl)methyl)phenyl)amino)-3,3-dicyclopropyl-1-oxopropan-2-yl) carbamate (125 mg, 245 μmol, 49.8% yield) as a white solid. LCMS [M+H]⁺=509.4 m/z.

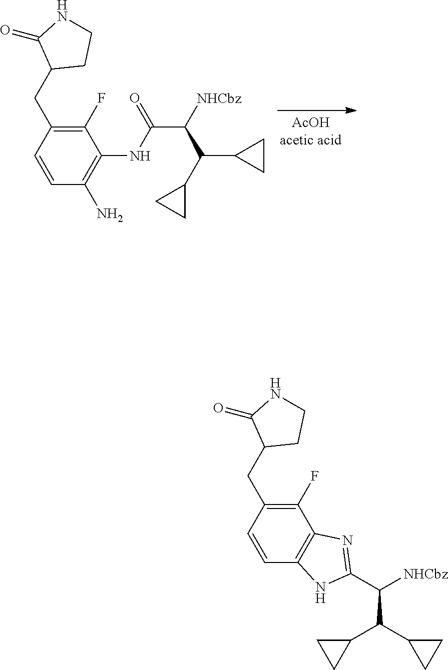 [SMILES: Nc1ccc(CC2CCNC2=O)c(F)c1NC(=O)[C@@H](NC(=O)OCc1ccccc1)C(C1CC1)C1CC1 ; O=C(N[C@H](c1nc2c(F)c(CC3CCNC3=O)ccc2[nH]1)C(C1CC1)C1CC1)OCc1ccccc1]

Step 8: A solution of benzyl ((2S)-1-((6-amino-2-fluoro-3-((2-oxopyrrolidin-3-yl)methyl)phenyl)amino)-3,3-dicyclopropyl-1-oxopropan-2-yl) carbamate (125 mg, 245 μmol, 1.00 eq) in AcOH (14.7 mg, 245 μmol, 14.0 μL, 1.00 eq) was stirred at 70° C. for 2 h. The reaction mixture was diluted with water and extracted with EtOAc. The combined organic layers were washed with sat. aq NaHCO₃, dried over Na₂SO₄, filtered and concentrated under reduced pressure to give a residue, which was purified by prep-TLC (SiO₂, DCM:MeOH=10:1) to afford benzyl ((1S)-2,2-dicyclopropyl-1-(4-fluoro-5-((2-oxopyrrolidin-3-yl)methyl)-1H-benzo[d]imidazol-2-yl)ethyl) carbamate (70.0 mg, 142 μmol, 58.0% yield) as a white solid. LCMS [M+H]⁺=491.4 m/z.

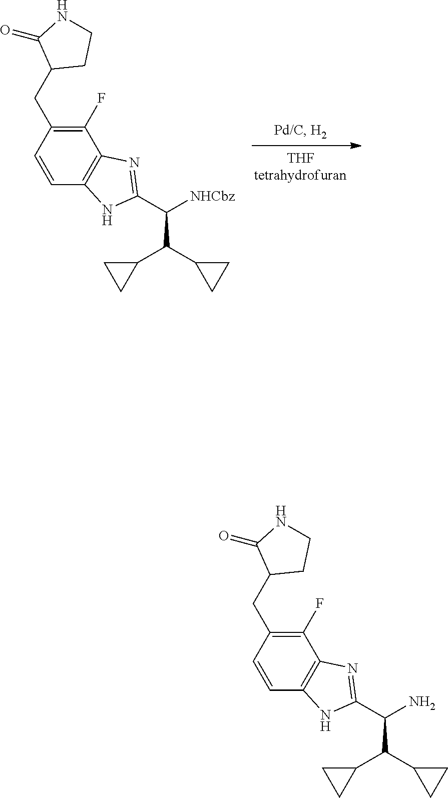 [SMILES: O=C(N[C@H](c1nc2c(F)c(CC3CCNC3=O)ccc2[nH]1)C(C1CC1)C1CC1)OCc1ccccc1 ; N[C@H](c1nc2c(F)c(CC3CCNC3=O)ccc2[nH]1)C(C1CC1)C1CC1]

Step 9: To a solution of benzyl ((1S)-2,2-dicyclopropyl-1-(4-fluoro-5-((2-oxopyrrolidin-3-yl)methyl)-1H-benzo[d]imidazol-2-yl)ethyl) carbamate (70.0 mg, 142 μmol, 1.00 eq) in THF (5.00 mL) was added Pd/C (7.00 mg, 10.0% purity) under nitrogen. The suspension was degassed and purged with hydrogen gas three times. The mixture was stirred under hydrogen (15.0 Psi) at RT for 2 h. The reaction mixture was filtered, the cake was washed with MeOH, and the filtrate was concentrated under reduced pressure to afford 3-((2-((S)-1-amino-2,2-dicyclopropylethyl)-4-fluoro-1H-benzo[d]imidazol-5-yl)methyl) pyrrolidin-2-one (50.0 mg, 140 μmol, 98.3% yield) as a white solid. LCMS [M+H]⁺=357.2 m/z.

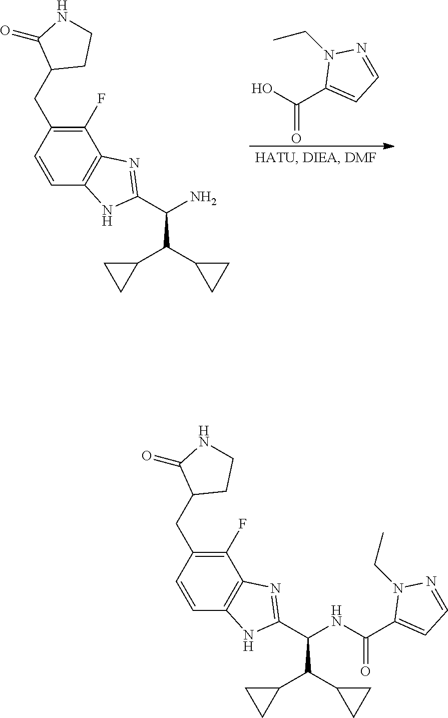

Step 10: To a solution of 3-((2-((S)-1-amino-2,2-dicyclopropylethyl)-4-fluoro-1H-benzo[d]imidazol-5-yl)methyl) pyrrolidin-2-one (40.0 mg, 112 μmol, 1.00 eq) and 1-ethyl-1H-pyrazole-5-carboxylic acid (23.5 mg, 168 μmol, 1.50 eq) in DCM (5.00 mL) was added HATU (128 mg, 336 μmol, 3.00 eq) and DIEA (72.5 mg, 561 μmol, 97.7 μL, 5.00 eq). The mixture was stirred at RT for 12 h. The reaction mixture was diluted with water and extracted with EtOAc. The combined organic layers were washed with sat. aq. NaHCO₃, dried over Na₂SO₄, filtered, and concentrated under reduced pressure to give a residue. The residue was purified by prep-TLC (SiO₂, DCM:MeOH=10:1) to afford N-((1S)-2,2-dicyclopropyl-1-(4-fluoro-5-((2-oxopyrrolidin-3-yl)methyl)-1H-benzo[d]imidazol-2-yl)ethyl)-1-ethyl-1H-pyrazole-5-carboxamide (26.9 mg, 51.4 μmol, 45.8% yield) as a white solid. H NMR δ 7.52 (d, J=2.0 Hz, 1H), 7.31-7.25 (m, 1H), 7.15 (t, J=6.8 Hz, 1H), 6.96 (s, 1H), 5.56 (d, J=6.8 Hz, 1H), 4.56-4.51 (m, 2H), 3.25-3.20 (m, 3H), 2.80-2.74 (m, 2H), 2.13-2.11 (m, 1H), 1.89-1.87 (m, 1H), 1.36 (t, J=3.2 Hz, 3H), 0.85-0.75 (m, 3H), 0.49-0.40 (m, 3H), 0.26-0.10 (m, 4H), −0.10-−0.15 (m, 1H). LCMS [M+H]⁺=479.4 m/z. The title compound was isolated as the second eluting, single stereoisomer by chiral SFC purification (column: Phenomenex Cellulose-2 (250 mm×30 mm, 10 μm); mobile phase: 30% [0.1% NH₃H₂O in MeOH]). H NMR δ 7.53 (d, J=2.0 Hz, 1H), 7.28-7.26 (m, 1H), 7.15 (t, J=6.8 Hz, 1H), 6.96 (d, J=2.0 Hz, 1H), 5.56 (d, J=6.8 Hz, 1H), 4.54-4.52 (m, 2H), 3.30-3.23 (m, 3H), 2.83-2.74 (m, 2H), 2.13-2.09 (m, 1H), 1.89-1.86 (m, 1H), 1.36 (t, J=7.2 Hz, 3H), 0.89-0.75 (m, 2H), 0.50-0.35 (m, 3H), 0.25-0.22 (m, 3H), 0.15-0.11 (m, 1H), −0.10-−0.15 (m, 1H). LCMS [M+H]⁺=479.4 m/z. Compound 2 was isolated as mixture of isomers before SFC purification.

### Example 2: Exemplary Synthesis of Compounds 3-5

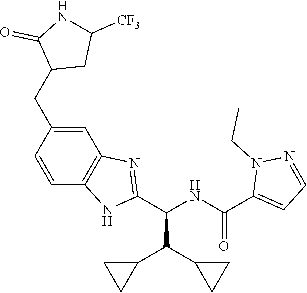 [SMILES: CCn1nccc1C(=O)N[C@H](c1nc2cc(CC3CC(C(F)(F)F)NC3=O)ccc2[nH]1)C(C1CC1)C1CC1]

### N-((1S)-2,2-dicyclopropyl-1-(5-((2-oxo-5-(trifluoromethyl) pyrrolidin-3-yl)methyl)-1H-benzo[d]imidazol-2-yl)ethyl)-1-ethyl-1H-pyrazole-5-carboxamide (3-5)

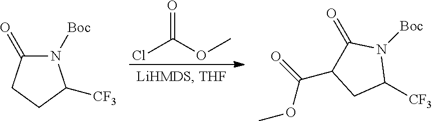

Step 1: To a solution of tert-butyl 2-oxo-5-(trifluoromethyl) pyrrolidine-1-carboxylate (10.0 g, 39.4 mmol, 1.00 eq) in THF (100 mL) was added dropwise LiHMDS (1 M, 82.9 mL, 2.10 eq) at −70° C. After addition, the mixture was stirred at −70° C. for 0.5 h, before adding methyl carbonochloridate (6.34 g, 67.1 mmol, 5.20 mL, 1.70 eq), dropwise. The resulting mixture was stirred at −70° C. for 1 h. The reaction mixture was diluted with water, extracted with EtOAc, dried with anhydrous Na₂SO₄, filtrated, and concentrated under reduced pressure to afford 1-(tert-butyl) 3-methyl 2-oxo-5-(trifluoromethyl) pyrrolidine-1,3-dicarboxylate (5.00 g, 16.0 mmol, 40.6% yield) as a yellow oil.

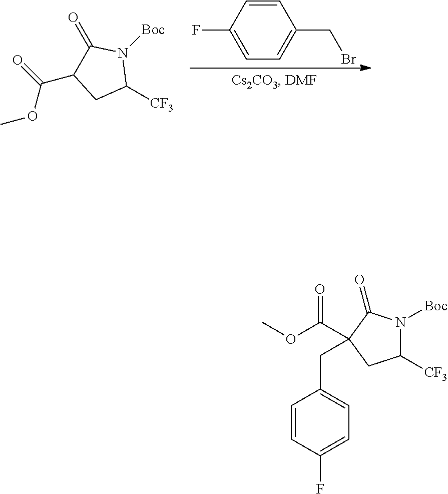 [SMILES: COC(=O)C1CC(C(F)(F)F)N(C(=O)OC(C)(C)C)C1=O ; Fc1ccc(CBr)cc1 ; COC(=O)C1(Cc2ccc(F)cc2)CC(C(F)(F)F)N(C(=O)OC(C)(C)C)C1=O]

Step 2: To a solution of 1-(tert-butyl) 3-methyl 2-oxo-5-(trifluoromethyl) pyrrolidine-1,3-dicarboxylate (5.00 g, 16.0 mmol, 1.00 eq) and 1-(bromomethyl)-4-fluorobenzene (3.64 g, 19.2 mmol, 2.38 mL, 1.20 eq) in DMF (1.00 mL) was added Cs₂CO₃ (15.7 g, 48.1 mmol, 3.00 eq). The mixture was stirred at RT for 2 h. The reaction mixture was diluted with H₂O, extracted with EtOAc, and dried over anhydrous Na₂SO₄, The filtrate was concentrated under reduced pressure, and the residue was purified by column chromatography (SiO₂, petroleum ether: EtOAc) to afford 1-(tert-butyl) 3-methyl 3-(4-fluorobenzyl)-2-oxo-5-(trifluoromethyl) pyrrolidine-1,3-dicarboxylate (3.00 g, 7.15 mmol, 44.5% yield) as a white solid.

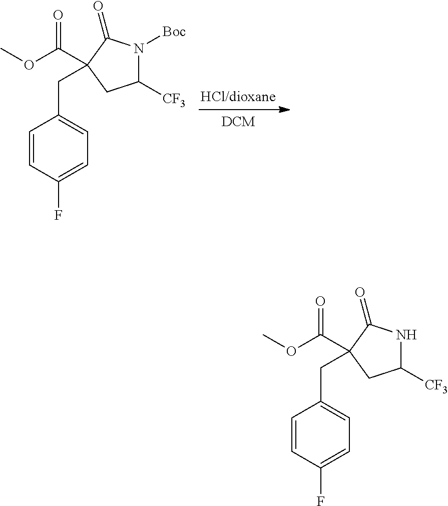 [SMILES: COC(=O)C1(Cc2ccc(F)cc2)CC(C(F)(F)F)N(C(=O)OC(C)(C)C)C1=O ; COC(=O)C1(Cc2ccc(F)cc2)CC(C(F)(F)F)NC1=O]

Step 3: To a solution of 1-(tert-butyl) 3-methyl 3-(4-fluorobenzyl)-2-oxo-5-(trifluoromethyl) pyrrolidine-1,3-dicarboxylate (3.00 g, 7.15 mmol, 1.00 eq) in DCM (20.0 mL) was added HCl/dioxane (4 M, 12.0 mL, 6.71 eq). The mixture was stirred at RT for 2 h. The reaction mixture was concentrated in under vacuum to afford methyl 3-(4-fluorobenzyl)-2-oxo-5-(trifluoromethyl) pyrrolidine-3-carboxylate (2.50 g, 7.03 mmol, 98.2% yield, HCl) as a yellow oil. LCMS [M+H]⁺=320.0 m/z.

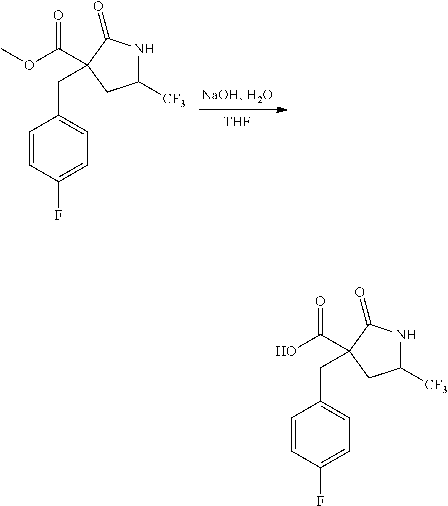 [SMILES: COC(=O)C1(Cc2ccc(F)cc2)CC(C(F)(F)F)NC1=O ; O=C(O)C1(Cc2ccc(F)cc2)CC(C(F)(F)F)NC1=O ; "THF"]

Step 4: To a solution of methyl 3-(4-fluorobenzyl)-2-oxo-5-(trifluoromethyl) pyrrolidine-3-carboxylate (2.50 g, 7.03 mmol, 1.00 eq, HCl) in THF (20.0 mL) was added NaOH (562 mg, 14.0 mmol, 2.00 eq) and H₂O (5.00 mL) at 0° C. The mixture was stirred at RT for 2 h. The reaction mixture was diluted with H₂O (20.0 mL), then adjusted with 1 M HCl to pH=5 and extracted with EtOAc. The combined organic layers were dried over anhydrous Na₂SO₄, and the filtrate was concentrated under reduced pressure to give a residue. The residue was purified by prep-HPLC (FA condition; column: Phenomena Luna C18 (250*70 mm, 10 μm); mobile phase: [water (FA)-ACN]; B %: 30%-60%, 21 min) to afford 3-(4-fluorobenzyl)-2-oxo-5-(trifluoromethyl) pyrrolidine-3-carboxylic acid (1.50 g, 4.91 mmol, 69.9% yield) as a white solid. H NMR (400 MHZ, CDCl₃) δ 7.16-7.02 (m, 4H), 7.00-6.95 (m, 1H), 3.74-2.69 (m, 2H), 2.44-2.38 (m, 1H), 0.94-0.85 (m, 1H).

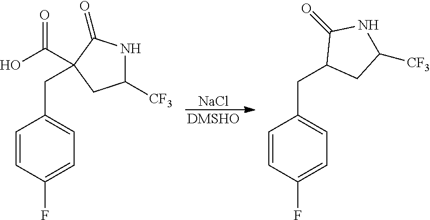

Step 5: To a solution of 3-(4-fluorobenzyl)-2-oxo-5-(trifluoromethyl) pyrrolidine-3-carboxylic acid (1.50 g, 4.91 mmol, 1.00 eq) in DMSO (15.0 mL) was added NaCl (574 mg, 9.83 mmol, 2.00 eq). The mixture was stirred at 130° C. for 2 h. The reaction mixture was diluted with H₂O, extracted with EtOAc, dried with anhydrous Na₂SO₄, filtrated, and concentrated under reduced pressure to afford 3-(4-fluorobenzyl)-5-(trifluoromethyl) pyrrolidin-2-one (1.00 g, 3.83 mmol, 77.9% yield) as yellow oil. LCMS [M+H]⁺=262.1 m/z.

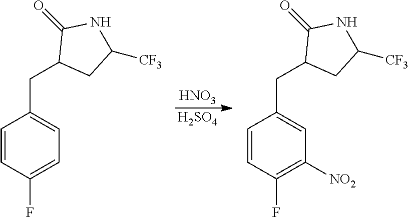

Step 6: To a solution of 3-(4-fluorobenzyl)-5-(trifluoromethyl) pyrrolidin-2-one (1.00 g, 3.83 mmol, 1.00 eq) in H₂SO₄ (10.0 mL) was added HNO₃ (689 mg, 7.66 mmol, 492 μL, 70.0% purity, 2.00 eq) at 0° C. The mixture was stirred at 0° C. for 2 h. The reaction was diluted with H₂O, extracted with EtOAc, dried with anhydrous Na₂SO₄, filtrated, and concentrated under reduced pressure to afford 3-(4-fluoro-3-nitrobenzyl)-5-(trifluoromethyl) pyrrolidin-2-one (1.00 g, 3.27 mmol, 85.3% yield) as yellow oil. LCMS [M+H]⁻=307.3 m/z.

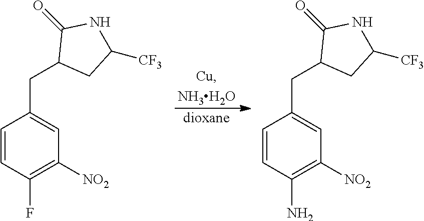

Step 7: To a solution of 3-(4-fluoro-3-nitrobenzyl)-5-(trifluoromethyl) pyrrolidin-2-one (1.00 g, 3.27 mmol, 1.00 eq) in dioxane (10.0 mL) was added Cu (207 mg, 3.27 mmol, 23.1 μL, 1.00 eq) and NH₃·H₂O (8.27 g, 59.0 mmol, 9.09 mL, 25.0% purity, 18.0 eq). The reaction mixture was stirred at 110° C. for 12 h. The reaction mixture was filtrated, diluted with H₂O, extracted with EtOAc, dried over anhydrous Na₂SO₄, filtrated, and concentrated under reduced pressure. The residue was purified by column chromatography (SiO₂, petroleum ether:ethyl acetate=10:1 to 1:1) to afford 3-(4-amino-3-nitrobenzyl)-5-(trifluoromethyl) pyrrolidin-2-one (500 mg, 1.65 mmol, 50.4% yield) as yellow solid. H NMR (400 MHZ, CDCl₃) δ 7.95 (t, J=1.6 Hz, 1H), 7.23 (t, J=1.6 Hz, 1H), 6.93-6.88 (m, 1H), 6.78 (t, J=8.4 Hz, 1H), 6.62 (dd, J₁=8.4 Hz, J₂=1.6 Hz, 1H), 6.52-6.49 (m, 1H), 4.13-4.03 (m, 1H), 2.80-2.65 (m, 3H), 2.43-2.32 (m, 2H).

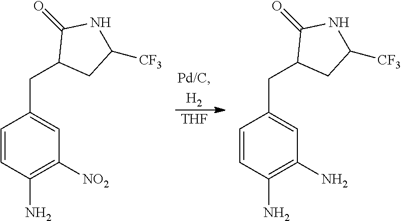 [SMILES: Nc1ccc(CC2CC(C(F)(F)F)NC2=O)cc1[N+](=O)[O-] ; "Pd/C,H2" ; Nc1ccc(CC2CC(C(F)(F)F)NC2=O)cc1N ; "THF"]

Step 8: To a solution of 3-(4-amino-3-nitrobenzyl)-5-(trifluoromethyl) pyrrolidin-2-one (500 mg, 1.65 mmol, 1.00 eq) in THF (4.00 mL) was added Pd/C (50.0 mg, 10% purity) under N₂. The suspension was degassed under vacuum and purged with H₂ three times. The mixture was stirred under H₂ (15 psi) at RT for 2 h. The reaction mixture was filtrated and concentrated under reduced pressure to afford 3-(3,4-diaminobenzyl)-5-(trifluoromethyl) pyrrolidin-2-one (400 mg, 1.46 mmol, 88.7% yield) as a yellow solid.

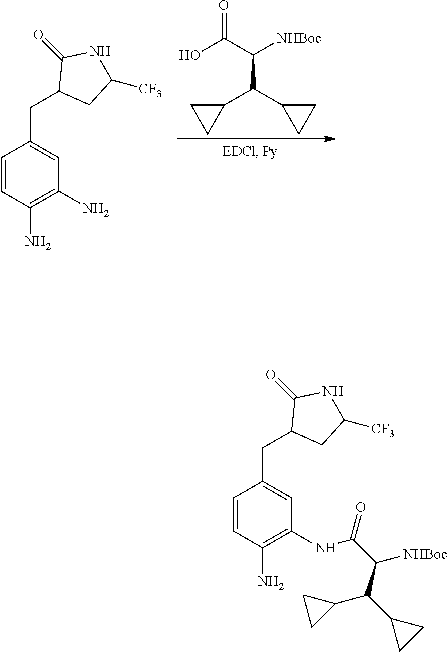 [SMILES: Nc1ccc(CC2CC(C(F)(F)F)NC2=O)cc1N ; CC(C)(C)OC(=O)N[C@H](C(=O)O)C(C1CC1)C1CC1 ; CC(C)(C)OC(=O)N[C@H](C(=O)Nc1cc(CC2CC(C(F)(F)F)NC2=O)ccc1N)C(C1CC1)C1CC1]

Step 9: To a solution of 3-(3,4-diaminobenzyl)-5-(trifluoromethyl) pyrrolidin-2-one (400 mg, 1.46 mmol, 1.00 eq), (S)-2-((tert-butoxycarbonyl)amino)-3,3-dicyclopropylpropanoic acid (197 mg, 731 μmol, 0.500 eq) in pyridine (3.00 mL) was added EDCI (561 mg, 2.93 mmol, 2.00 eq). The reaction mixture was stirred at RT for 2 h. The reaction mixture was diluted with H₂O, extracted with EtOAc, dried with anhydrous Na₂SO₄, filtrated, and concentrated under reduced pressure to afford tert-butyl ((2S)-1-((2-amino-5-((2-oxo-5-(trifluoromethyl) pyrrolidin-3-yl)methyl)phenyl)amino)-3,3-dicyclopropyl-1-oxopropan-2-yl) carbamate (500 mg, 953 μmol, 65.1% yield) as a yellow solid.

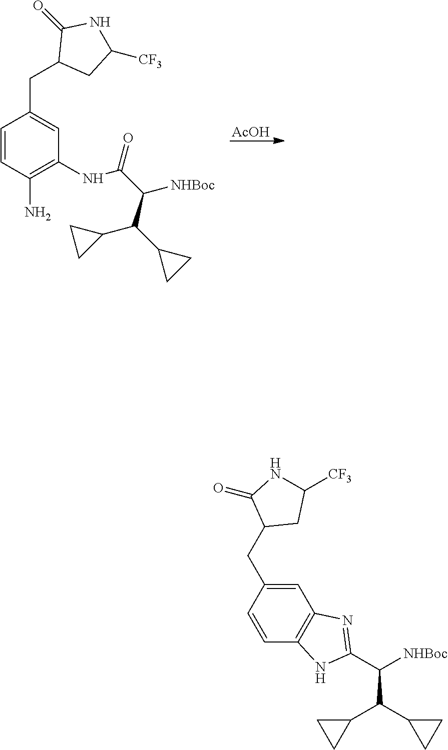 [SMILES: "AcOH" ; CC(C)(C)OC(=O)N[C@H](C(=O)Nc1cc(CC2CC(C(F)(F)F)NC2=O)ccc1N)C(C1CC1)C1CC1 ; CC(C)(C)OC(=O)N[C@H](c1nc2cc(CC3CC(C(F)(F)F)NC3=O)ccc2[nH]1)C(C1CC1)C1CC1]

Step 10: A mixture of tert-butyl ((2S)-1-((2-amino-5-((2-oxo-5-(trifluoromethyl) pyrrolidin-3-yl)methyl)phenyl)amino)-3,3-dicyclopropyl-1-oxopropan-2-yl) carbamate (500 mg, 953 μmol, 1.00 eq) in AcOH (3.00 mL) was stirred at 70° C. for 2 h. The reaction mixture was concentrated under reduced pressure. The crude residue was diluted with sat. aq. NaHCO₃, extracted with EtOAc, dried over anhydrous Na₂SO₄, filtered, and concentrated under reduced pressure to give a residue. The residue was purified by prep-HPLC (neutral condition; column: Waters Xbridge (150*25 m, 5 μm); mobile phase: [water (NH₄HCO₃)-ACN]; B %: 41%-71%) to afford tert-butyl ((1S)-2,2-dicyclopropyl-1-(5-((2-oxo-5-(trifluoromethyl) pyrrolidin-3-yl)methyl)-1H-benzo[d]imidazol-2-yl)ethyl) carbamate (200 mg, 394.82 μmol, 41.42% yield) as a yellow solid. LCMS [M+H]⁺=507.4 m/z.

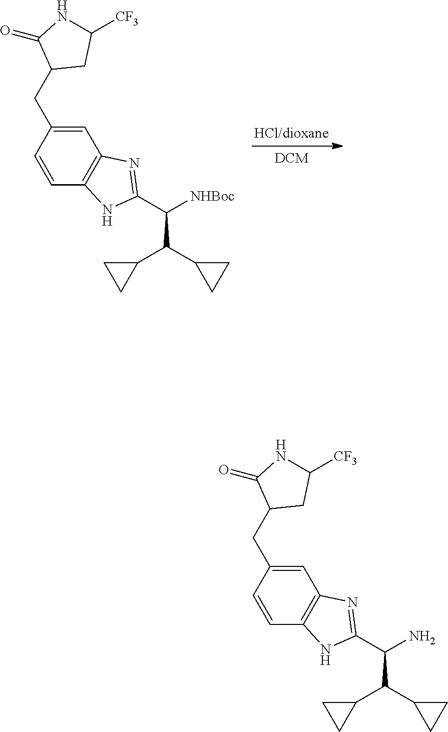 [SMILES: CC(C)(C)OC(=O)N[C@H](c1nc2cc(CC3CC(C(F)(F)F)NC3=O)ccc2[nH]1)C(C1CC1)C1CC1 ; N[C@H](c1nc2cc(CC3CC(C(F)(F)F)NC3=O)ccc2[nH]1)C(C1CC1)C1CC1]

Step 11: To a solution of tert-butyl ((1S)-2,2-dicyclopropyl-1-(5-((2-oxo-5-(trifluoromethyl) pyrrolidin-3-yl)methyl)-1H-benzo[d]imidazol-2-yl)ethyl) carbamate (200 mg, 394 μmol, 1.00 eq) in DCM (1.00 mL) was added HCl/dioxane (4 M, 1 mL, 10.1 eq). The reaction mixture was stirred at RT for 2 h. The reaction mixture was concentrated under reduced pressure to afford 3-((2-((S)-1-amino-2,2-dicyclopropylethyl)-1H-benzo[d]imidazol-5-yl)methyl)-5-(trifluoromethyl) pyrrolidin-2-one (170 mg, 383 μmol, 97.2% yield, HCl) as a yellow solid. LCMS [M+H]⁺=407.1 m/z.

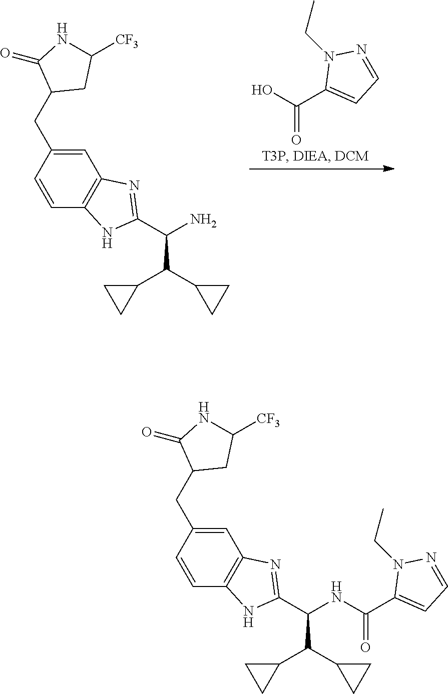

Step 12: To a solution of 3-((2-((S)-1-amino-2,2-dicyclopropylethyl)-1H-benzo[d]imidazol-5-yl)methyl)-5-(trifluoromethyl) pyrrolidin-2-one (170 mg, 418 μmol, 1.00 eq) and 1-ethyl-1H-pyrazole-5-carboxylic acid (64.4 mg, 460 μmol, 1.10 eq) in DCM (3.00 mL) was added T3P (532 mg, 836 μmol, 497 μL, 50.0% purity, 2.00 eq) and DIEA (162 mg, 1.25 mmol, 218 μL, 3.00 eq). The mixture was stirred at RT for 2 h. The reaction mixture was diluted with H₂O, extracted with EtOAc, dried with anhydrous Na₂SO₄, filtrated, and concentrated under reduced pressure. The residue was purified by prep-HPLC (neutral condition; column: Waters Xbridge (150*25 mm, 5 μm); mobile phase: [water (NH₄HCO₃)-ACN]; B %: 36%-66%) to afford N-((1S)-2,2-dicyclopropyl-1-(5-((2-oxo-5-(trifluoromethyl) pyrrolidin-3-yl)methyl)-1H-benzo[d]imidazol-2-yl)ethyl)-1-ethyl-1H-pyrazole-5-carboxamide (100 mg, 189 μmol, 45.2% yield) as a white solid. Example 3 was isolated as the third eluting, single stereoisomer by chiral SFC purification (column: Diacel Chiralcel OD (250 mm×30 mm, 10 μm); mobile phase: 25% [0.1% NH₃H₂O in IPA]); (58.24 mg, 109 μmol, 44.4% yield, 99.2% purity). LCMS [M+H]⁺=529.3 m/z. Example 4 is the first eluting, single stereoisomer, and Example 5 is the second eluting, single stereoisomer.

Table 1 includes spectroscopic data for compounds synthesized as described in Examples 1-2 or analogously synthesized using the exemplary procedures of Examples 1-2. Compounds 2 and 9 were isolated as a mixture of isomers.

TABLE 1
Spectroscopic data of Compounds 1-88

| DICE | Compound | | |
|---|---|---|---|
| ID | No. | LCMS | Structure |
| 18434 | 1 | 479.4 | 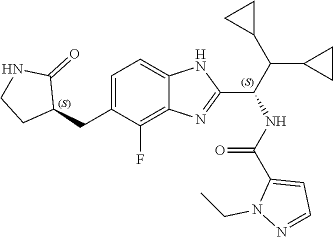 |
| 18433 | 2 | 479.4 | 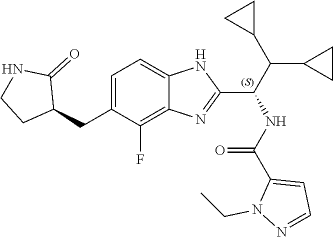 |
| 18442 | 3 | 529.3 | 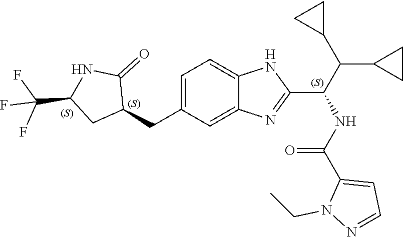 |
| 18439 | 4 | 529.2 | 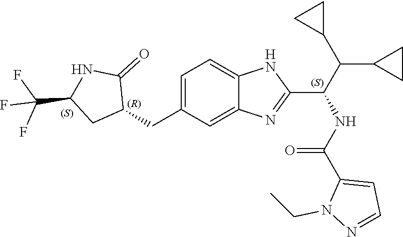 |
| 18441 | 5 | 529.3 | 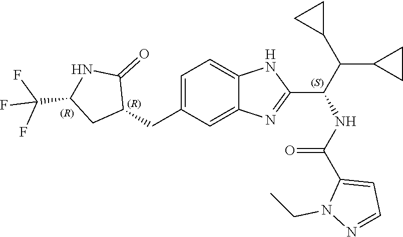 |
| 16733 | 6 | 463.2 | 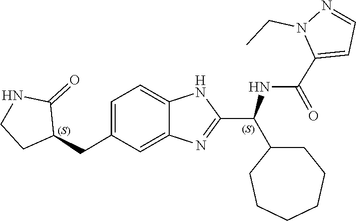 |
| 18447 | 7 | 485.3 | 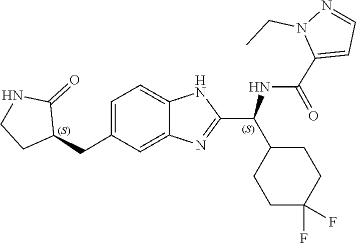 |
| 18448 | 8 | 485.3 | 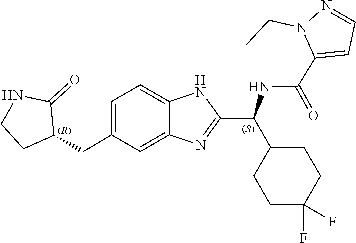 |
| 18452 | 9 | 486.3 | 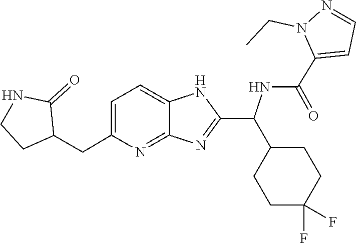 [SMILES: CCn1nccc1C(=O)NC(c1nc2nc(CC3CCNC3=O)ccc2[nH]1)C1CCC(F)(F)CC1] |
| 18456 | 10 | 486.4 | 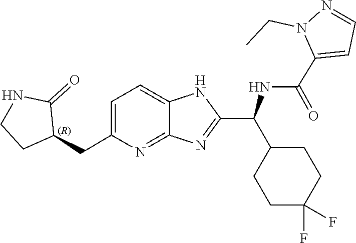 |
| 18453 | 11 | 503.2 | 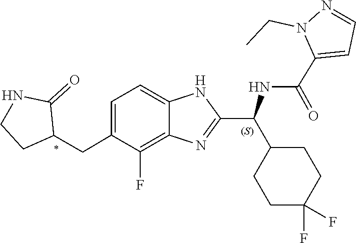 |
| | | | Single diasteromer with undefined |
| | | | absolute stereochemistry at * |
| 18457 | 12 | 503.4 | 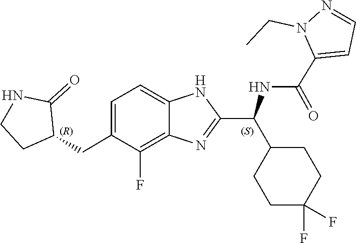 |
| 18455 | 13 | 503.4 | 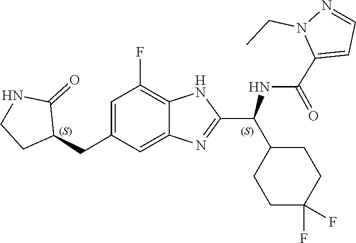 |
| 18459 | 14 | 503.4 | 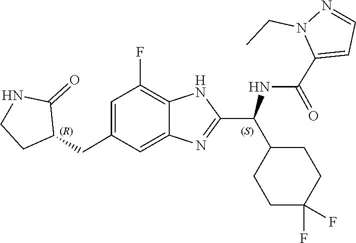 |
| 18473 | 15 | 461.3 | 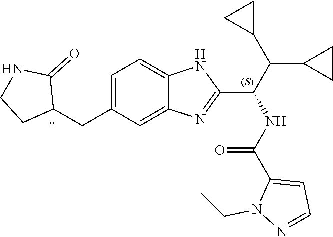 |
| | | | Single diasteromer with undefined |
| | | | absolute stereochemistry at * |
| 18474 | 16 | 461.3 | 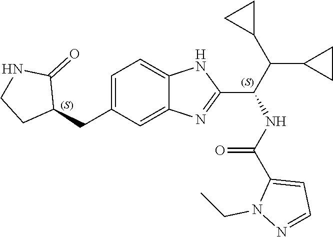 |
| 18502 | 17 | 475.3 | 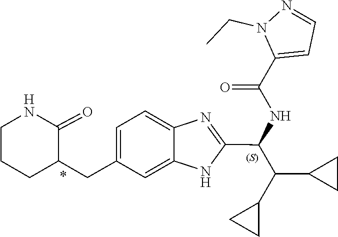 |
| | | | Single diasteromer with undefined |
| | | | absolute stereochemistry at * |
| 18503 | 18 | 475.3 | 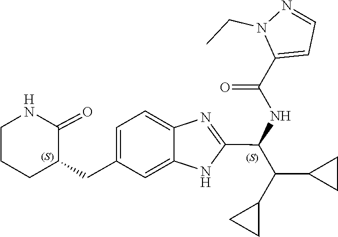 |
| 18508 | 19 | 475.3 | 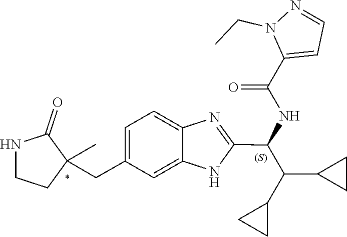 |
| | | | Single diasteromer with undefined |
| | | | absolute stereochemistry at * |
| 18509 | 20 | 475.3 | 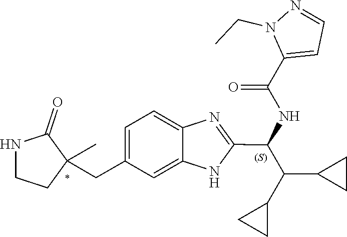 |
| | | | Single diasteromer with undefined |
| | | | absolute stereochemistry at * |
| 18510 | 21 | 475.4 | 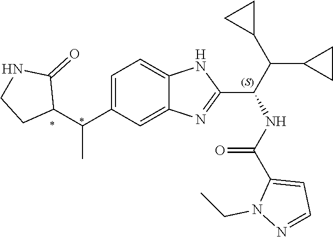 |
| | | | Single diasteromer with undefined |
| | | | absolute stereochemistry at * |
| 18511 | 22 | 475.4 | 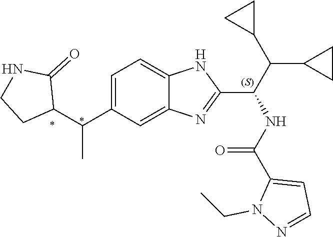 |
| | | | Single diasteromer with undefined |
| | | | absolute stereochemistry at * |
| 18512 | 23 | 475.3 | 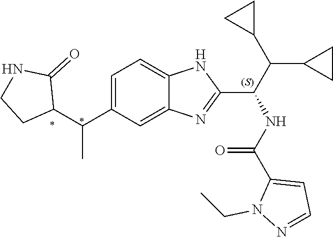 |
| | | | Single diasteromer with undefined |
| | | | absolute stereochemistry at * |
| 18513 | 24 | 475.3 | 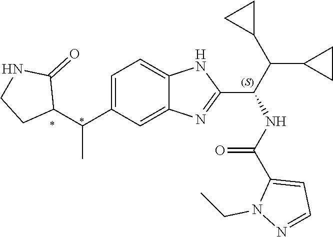 |
| | | | Single diasteromer with undefined |
| | | | absolute stereochemistry at * |
| 18648 | 25 | 487.4 | 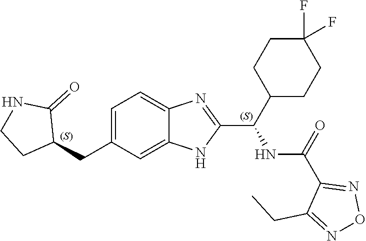 |
| 18649 | 26 | 499.4 | 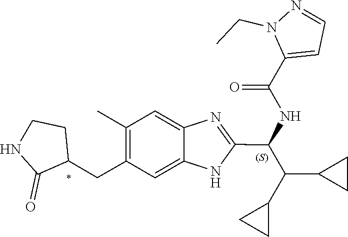 |
| | | | Single diasteromer with undefined |
| | | | absolute stereochemistry at * |
| 18778 | 27 | 461.3 | 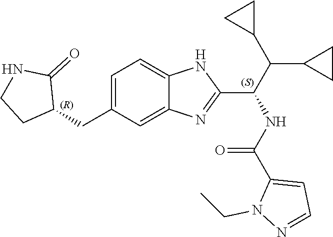 |
| 18815 | 28 | 486.4 | 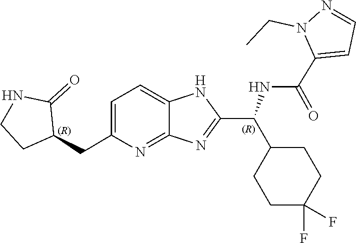 |
| 18816 | 29 | 486.4 | 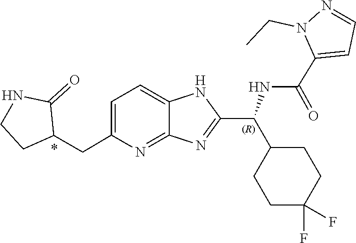 |
| | | | Single diasteromer with undefined |
| | | | absolute stereochemistry at * |
| 18848 | 30 | 486.4 | 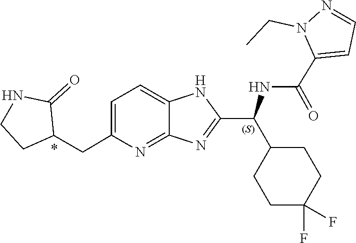 |
| | | | Single diasteromer with undefined |
| | | | absolute stereochemistry at * |
| 18850 | 31 | 479.4 | 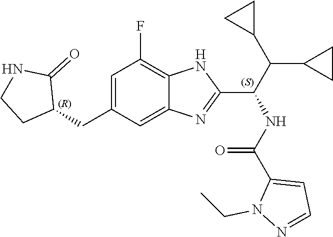 |
| 18851 | 32 | 479.3 | 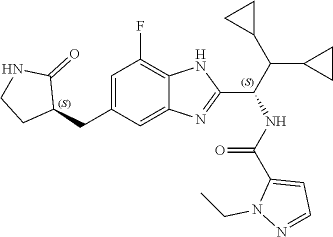 |
| 18852 | 33 | 547.3 | 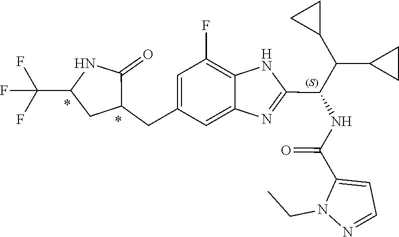 |
| | | | Single diasteromer with undefined |
| | | | absolute stereochemistry at * |
| 18853 | 34 | 547.3 | 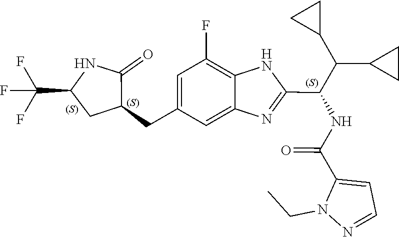 |
| 18877 | 35 | 475.3 | 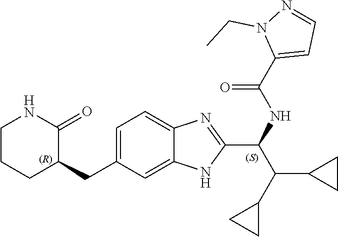 |
| 18881 | 36 | 479.4 | 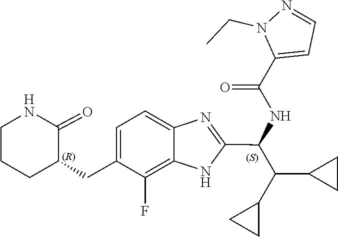 |
| 18882 | 37 | 547.3 | 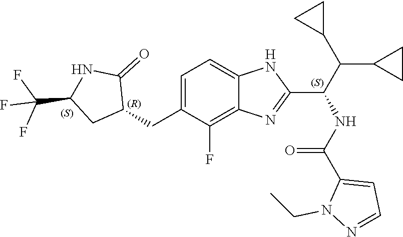 |
| 18883 | 38 | 547.3 | 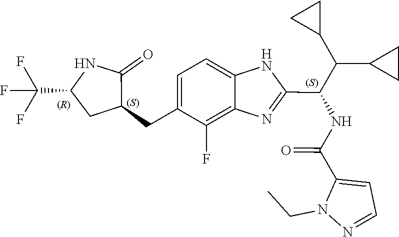 |
| 18884 | 39 | 547.3 | 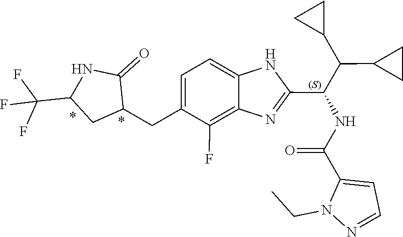 |
| | | | Single diasteromer with undefined |
| | | | absolute stereochemistry at * |
| 18885 | 40 | 547.3 | 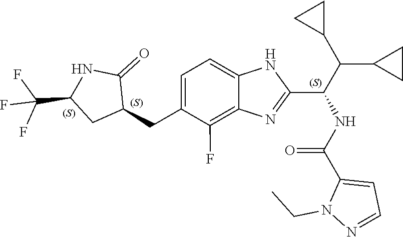 |
| 18886 | 41 | 547.3 | 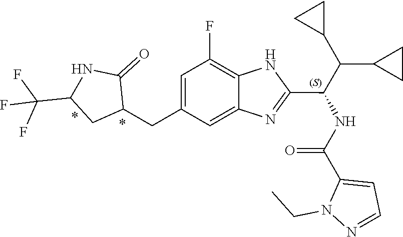 |
| | | | Single diasteromer with undefined |
| | | | absolute stereochemistry at * |
| 18887 | 42 | 547.3 | 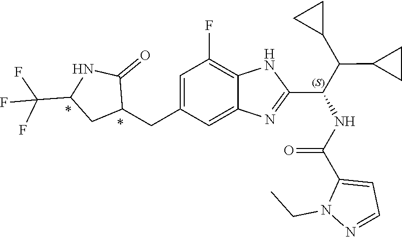 |
| | | | Single diasteromer with undefined |
| | | | absolute stereochemistry at * |
| 18892 | 43 | 543.3 | 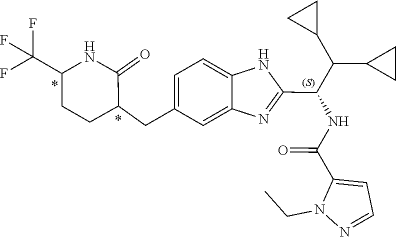 |
| | | | Single diasteromer with undefined |
| | | | absolute stereochemistry at * |
| 18893 | 44 | 543.3 | 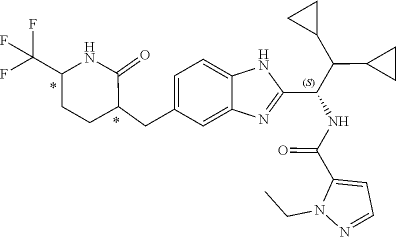 |
| | | | Single diasteromer with undefined |
| | | | absolute stereochemistry at * |
| 18894 | 45 | 543.4 | 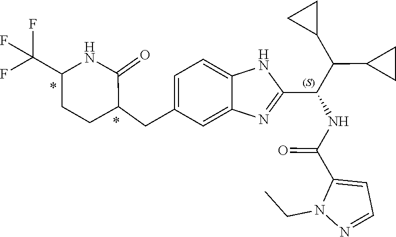 |
| | | | Single diasteromer with undefined |
| | | | absolute stereochemistry at * |
| 18895 | 46 | 543.4 | 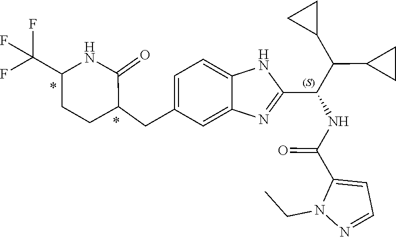 |
| | | | Single diasteromer with undefined |
| | | | absolute stereochemistry at * |
| 18896 | 47 | 561.3 | 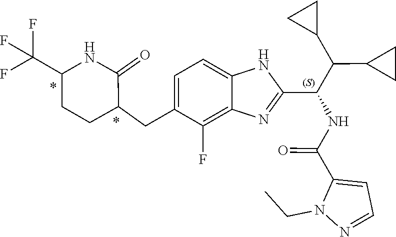 |
| | | | Single diasteromer with undefined |
| | | | absolute stereochemistry at * |
| 18897 | 48 | 561.3 | 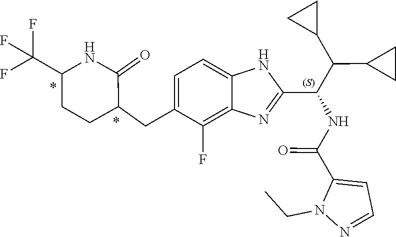 |
| | | | Single diasteromer with undefined |
| | | | absolute stereochemistry at * |
| 18898 | 49 | 561.3 | 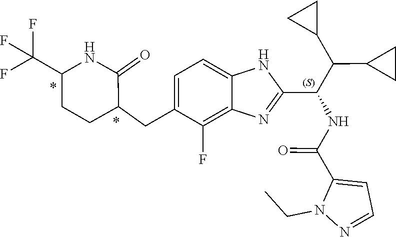 |
| | | | Single diasteromer with undefined |
| | | | absolute stereochemistry at * |
| 18899 | 50 | 561.2 | 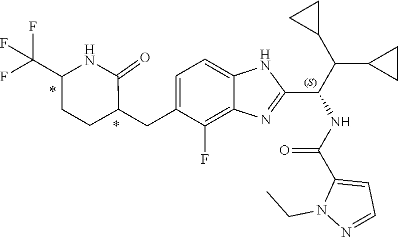 |
| | | | Single diasteromer with undefined |
| | | | absolute stereochemistry at * |
| 18908 | 51 | 561.5 | 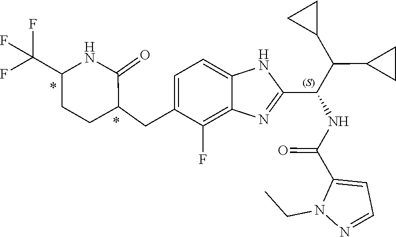 |
| | | | Single diasteromer with undefined |
| | | | absolute stereochemistry at * |
| 18900 | 52 | 561.3 | 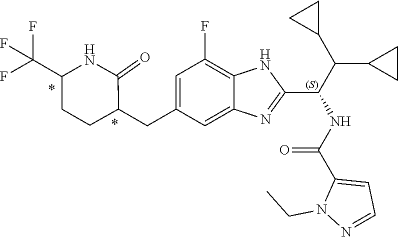 |
| | | | Single diasteromer with undefined |
| | | | absolute stereochemistry at * |
| 18901 | 53 | 561.3 | 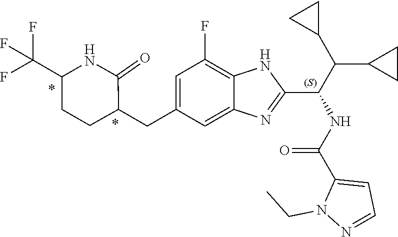 |
| | | | Single diasteromer with undefined |
| | | | absolute stereochemistry at * |
| 18902 | 54 | 561.3 | 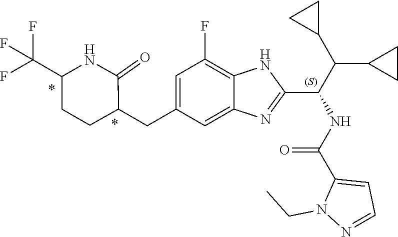 |
| | | | Single diasteromer with undefined |
| | | | absolute stereochemistry at * |
| 18903 | 55 | 561.3 | 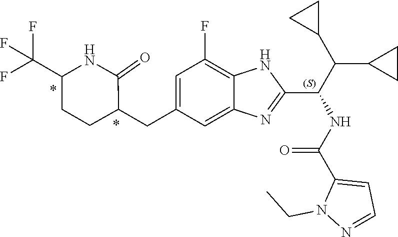 |
| | | | Single diasteromer with undefined |
| | | | absolute stereochemistry at * |
| 18909 | 56 | 561.3 | 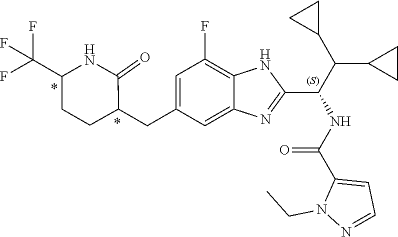 |
| | | | Single diasteromer with undefined |
| | | | absolute stereochemistry at * |
| 18959 | 57 | 486.3 | 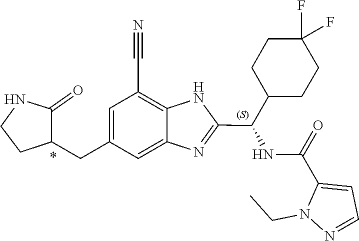 |
| | | | Single diasteromer with undefined |
| | | | absolute stereochemistry at * |
| 19123 | 58 | 475.3 | 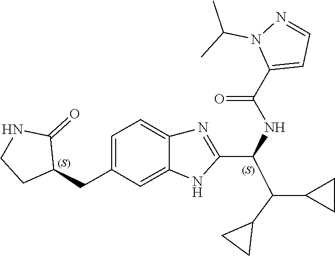 |
| 19124 | 59 | 447.3 | 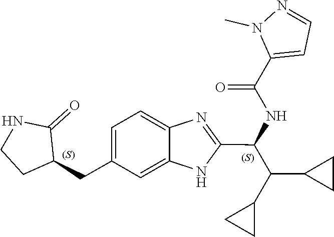 |
| 19125 | 60 | 463.3 | 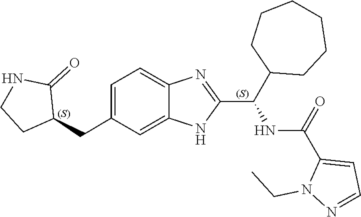 |
| 19126 | 61 | 463.4 | 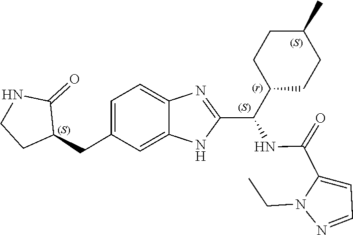 |
| 19127 | 62 | 533.4 | 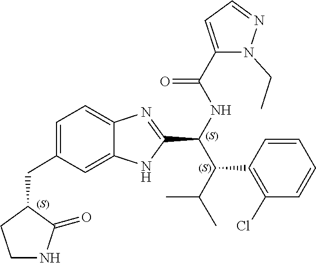 |
| 19128 | 63 | 503.3 | 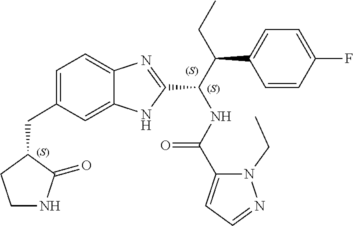 |
| 19130 | 64 | 489.3 | 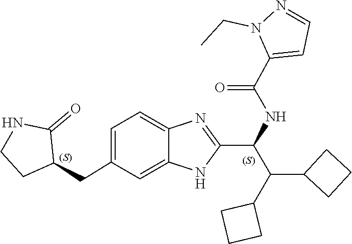 |
| 19132 | 65 | 463.2 | 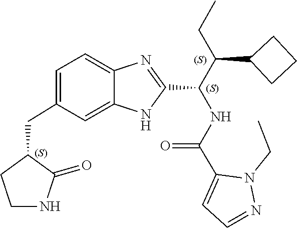 |
| 19133 | 66 | 491.5 | 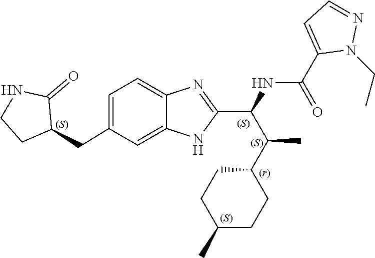 |
| 19135 | 67 | 449.0 | 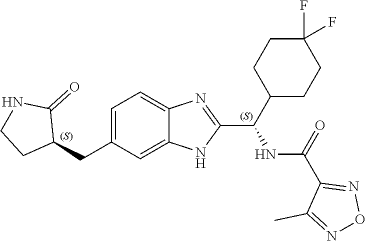 |
| 19155 | 68 | 493.2 | 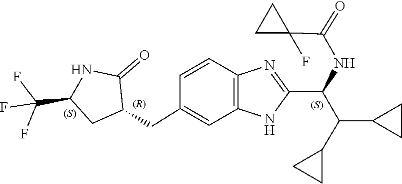 |
| 19156 | 69 | 493.3 | 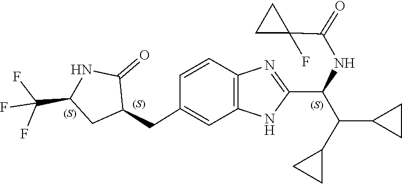 |
| 19173 | 70 | 489.4 | 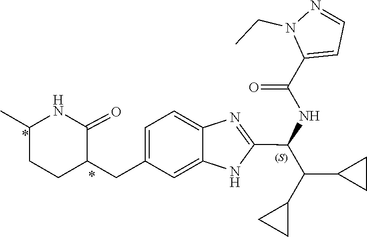 |
| | | | Single diasteromer with undefined |
| | | | absolute stereochemistry at * |
| 19174 | 71 | 543.4 | 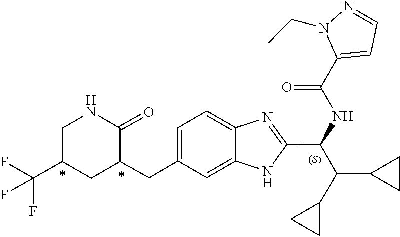 |
| | | | Single diasteromer with undefined |
| | | | absolute stereochemistry at * |
| 19175 | 72 | 475.2 | 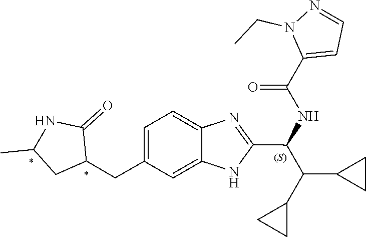 |
| | | | Single diasteromer with undefined |
| | | | absolute stereochemistry at * |
| 19177 | 73 | 511.2 | 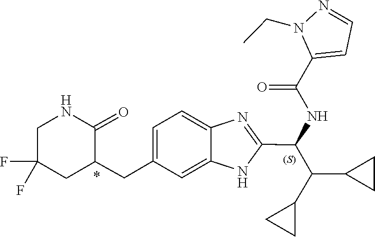 |
| | | | Single diasteromer with undefined |
| | | | absolute stereochemistry at * |
| 19437 | 74 | 511.3 | 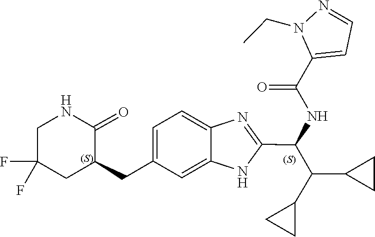 |
| 19438 | 75 | 511.3 | 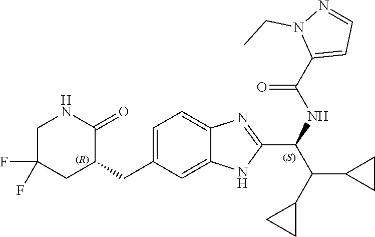 |
| 19178 | 76 | 473.2 | 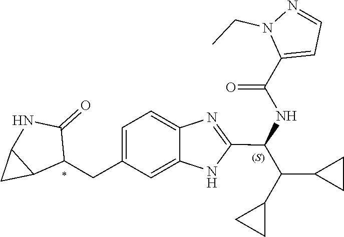 |
| | | | Single diasteromer with undefined |
| | | | absolute stereochemistry at * |
| 19179 | 77 | 529.3 | 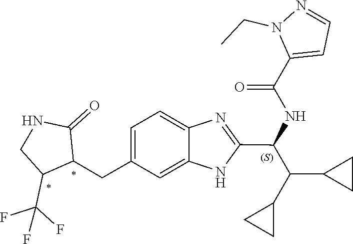 |
| | | | Single diasteromer with undefined |
| | | | absolute stereochemistry at * |
| 19278 | 78 | 573.3 | 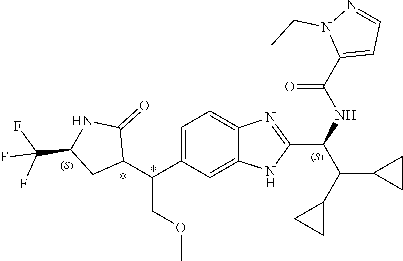 |
| | | | Single diasteromer with undefined |
| | | | absolute stereochemistry at * |
| 19283 | 79 | 573.2 | 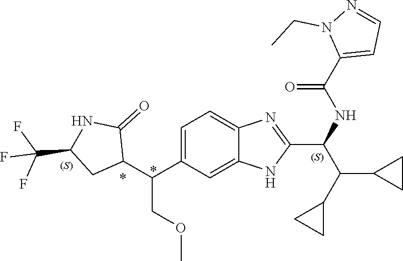 |
| | | | Single diasteromer with undefined |
| | | | absolute stereochemistry at * |
| 19284 | 80 | 573.1 | 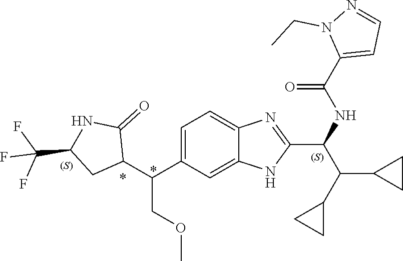 |
| | | | Single diasteromer with undefined |
| | | | absolute stereochemistry at * |
| 19443 | 81 | 489.3 | 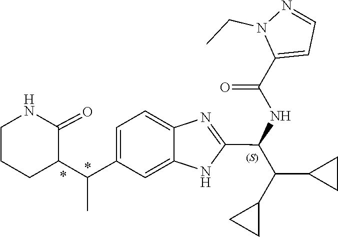 |
| | | | Single diasteromer with undefined |
| | | | absolute stereochemistry at * |
| 19446 | 82 | 489.4 | 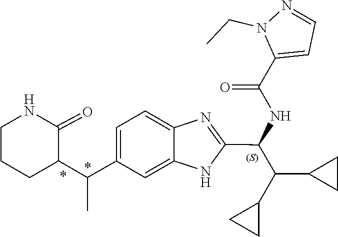 |
| | | | Single diasteromer with undefined |
| | | | absolute stereochemistry at * |
| 19447 | 83 | 489.4 | 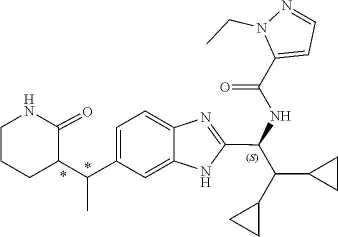 |
| | | | Single diasteromer with undefined |
| | | | absolute stereochemistry at * |
| 19448 | 84 | 489.3 | 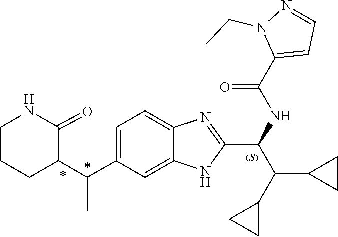 |
| | | | Single diasteromer with undefined |
| | | | absolute stereochemistry at * |
| 19449 | 85 | 489.3 | 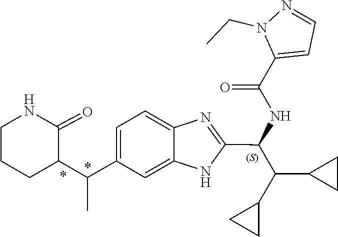 |
| | | | Single diasteromer with undefined |
| | | | absolute stereochemistry at * |
| 19444 | 86 | 499.3 | 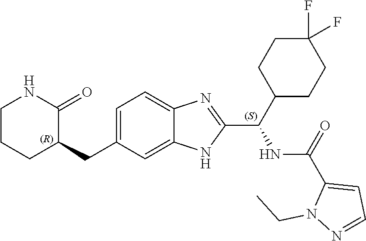 |
| 19445 | 87 | 477.3 | 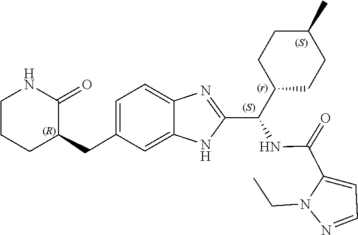 |
| 19460 | 88 | 531.3 | 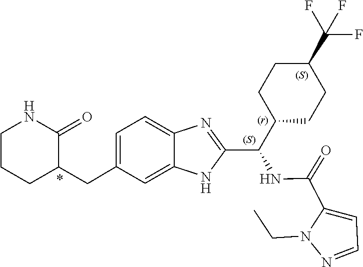 |
| | | | Single diasteromer with undefined |
| | | | absolute stereochemistry at * |

### Example 3: IL-17A/A HEK-Blue Cell Assay

The HEK-Blue IL-17A reporter cell line (Fisher #NC1408637) was used for cell-based IL-17A/A inhibition assays. Cells were grown and prepared for assays according to the manufacturer's instructions. This cell line consists of HEK 293 cells that were designed to expressed IL-17RA, IL-17RC, and the ActI adapter molecule, the combination of which, when stimulated by IL-17A/A activates a NFκB promoter and drives expression of a recombinant Secreted Alkaline Phosphatase (SEAP) gene protein. Media from the cells is then added to a development reagent (Quanti-Blue Substrate, Fisher #NC9711613), and read at A630.

Compounds were titrated in DMSO, with a top final compound concentration of 10 μM, 1 uM, or 0.3 μM, and added to the cells immediately before adding IL-17A/A (Genscript #Z03228). The cells, compound, and IL-17A/A were then incubated for 20 hours before media was removed for SEAP analysis. The resulting inhibition curve was then analyzed using Graphpad Prism 7.0, and IC₅₀ values were determined using a 4-parameter nonlinear fit. DMSO was added to a universal final concentration of 0.1% to optimize background.

Table 2 includes IC₅₀ values for IL-17A/A inhibition of selected compounds; with compounds having a IC₅₀ of A<100 nM; B 100-1000 nM; and C>1000 nM.

TABLE 2
IL-17 A/A Inhibition Data for selected compounds

| Compound | IL 17 |
|---|---|
| No. | IC₅₀ (nM) |
| 1 | B |
| 2 | B |
| 3 | A |
| 4 | B |
| 5 | C |
| 6 | A |
| 7 | B |
| 8 | C |
| 9 | C |
| 10 | C |
| 11 | B |
| 12 | C |
| 13 | B |
| 14 | C |
| 15 | B |
| 16 | B |
| 17 | B |
| 18 | C |
| 19 | C |
| 20 | B |
| 21 | B |
| 22 | C |
| 23 | C |
| 24 | C |
| 25 | B |
| 26 | C |
| 27 | C |
| 28 | C |
| 29 | C |
| 30 | C |
| 31 | C |
| 32 | B |
| 33 | B |
| 34 | A |
| 35 | B |
| 36 | C |
| 37 | B |
| 38 | B |
| 39 | C |
| 40 | A |
| 41 | B |
| 42 | C |
| 43 | B |
| 44 | C |
| 45 | B |
| 46 | C |
| 47 | B |
| 48 | C |
| 49 | C |
| 50 | A |
| 51 | B |
| 52 | B |
| 53 | B |
| 54 | C |
| 55 | C |
| 56 | B |
| 57 | B |
| 58 | B |
| 59 | B |
| 60 | A |
| 61 | A |
| 62 | C |
| 63 | C |
| 64 | B |
| 65 | B |
| 66 | B |
| 67 | B |
| 68 | C |
| 69 | B |
| 70 | B |
| 71 | B |
| 72 | B |
| 73 | B |
| 74 | A |
| 75 | B |
| 76 | B |
| 77 | B |
| 78 | C |
| 79 | B |
| 80 | B |
| 81 | C |
| 82 | B |
| 83 | C |
| 84 | B |
| 85 | C |
| 86 | C |
| 87 | C |
| 88 | C |

What is claimed is:

1. A compound of the Formula: 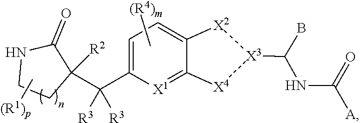
or a pharmaceutically acceptable salt thereof, wherein:
A is selected from 5- to 6-membered heteroaryl and C₃₋₆ carbocycle, any of which is optionally substituted with one or more substituents independently selected from:
halogen, —OR¹¹, —SR¹¹, —N(R¹¹)₂, —C(O)R¹¹, —C(O)N(R¹¹)₂, —N(R¹¹)C(O)R¹¹, —N(R¹¹)S(O)₂R¹¹, —C(O)OR¹¹, —OC(O)R¹¹, —S(O)R¹¹, —S(O)₂R¹¹, —NO₂, —CN;
and C₁₋₁₀ alkyl optionally substituted with one or more substituents independently selected from halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —S(O)R¹¹, —S(O)₂R¹¹, —NO₂, ═O, ═S, ═N(R¹¹), —CN, C₃₋₁₀ carbocycle and 3- to 10-membered heterocycle; wherein the C₃₋₁₀ carbocycle and 3- to 10-membered heterocycle are each optionally substituted with one or more substituents selected from:
halogen, —OR¹¹, —N(R¹¹)₂, —C(O)R¹¹, —C(O)N(R¹¹)₂, —(R¹¹)C(O)R¹¹, —C(O)OR¹¹, —OC(O)R¹¹, —NO₂, ═O, ═N(R¹¹), and —CN;
B is selected from —C(H)(R⁵)₂ and C₃₋₁₀ carbocycle, wherein each C₃₋₁₀ carbocycle is optionally substituted with one or more substituents independently selected from:
halogen, —OR¹², —SR¹², —N(R¹²)₂, —C(O)R¹², —C(O)N(R¹²)₂, —N(R¹²)C(O)R¹², —N(R¹²)S(O)₂R¹², —C(O)OR¹², —OC(O)R¹², —S(O)R¹², —S(O)₂R¹², —NO₂, ═O, ═S, ═N(R¹²), —CN; C₁₋₁₀ alkyl and C₃₋₁₀ carbocycle, any of which is optionally substituted with one or more substituents independently selected from halogen, —OR¹², —SR¹², —N(R¹²)₂, —C(O)R¹², —C(O)N(R¹²)₂, —N(R¹²)C(O)R¹², —C(O)OR¹², —OC(O)R¹², —S(O)R¹², —S(O)₂R¹², —NO₂, ═O, ═S, ═N(R¹²), —CN, C₃₋₁₀ carbocycle and 3- to 10-membered heterocycle; wherein the C₃₋₁₀ carbocycle and 3- to 10-membered heterocycle are each optionally substituted with one or more substituents selected from: halogen, —OR¹², —N(R¹²)₂, —C(O)R¹², —C(O)N(R¹²)₂, —N(R¹²)C(O)R¹², —C(O)OR¹², —OC(O)R¹², —NO₂, ═O, ═N(R¹²), and —CN;
each R⁵ is independently selected at each occurrence from (i), (ii), and (iii):
(i) halogen, —OR¹³, —SR¹³, —N(R¹³)₂, —C(O)R¹³, —C(O)N(R¹³)₂, —N(R¹³) C(O)R¹³, —N(R¹³)S(O)₂R¹³, —C(O)OR¹³, —OC(O)R¹³, —S(O)R¹³, —S(O)₂R¹³, —NO₂, ═O, ═S, ═N(R¹³), —CN;
(ii) C₁₋₁₀ alkyl optionally substituted with one or more substituents independently selected from halogen, —OR¹³, —SR¹³, —N(R¹³)₂, —C(O)R¹³, —C(O)N(R¹³)₂, —N(R¹³)C(O)R¹³, —N(R¹³)S(O)₂R¹³, —C(O)OR¹³, —OC(O)R¹³, —S(O)R¹³, —S(O)₂R¹³, —NO₂, ═O, ═S, ═N(R¹³), —CN; and
(iii) C₃₋₁₀ carbocycle optionally substituted with one or more substituents independently selected from:
halogen, —OR¹³, —SR¹³, —N(R¹³)₂, —C(O)R¹³, —C(O)N(R¹³)₂, —N(R¹³)C(O)R¹³, —N(R¹³)S(O)₂R¹³, —C(O)OR¹³, —OC(O)R¹³, —S(O)R¹³, —S(O)₂R¹³, —NO₂, ═O, ═S, ═N(R¹³), —CN; and C₁₋₁₀ alkyl optionally substituted with one or more substituents independently selected from halogen, —OR¹³, —SR¹³, —N(R¹³)₂, —C(O)R¹³, —C(O)N(R¹³)₂, —N(R¹³) C(O)R¹³, —C(O)OR¹³, —OC(O)R¹³, —S(O)R¹³, —S(O)₂R¹³, —NO₂, ═O, ═S, ═N(R¹³), and —CN;
each R¹ is independently selected at each occurrence from halogen, —OR¹⁴, —SR¹⁴, —N(R¹⁴)₂, —C(O)R¹⁴, —C(O)N(R¹⁴)₂, —N(R¹⁴)C(O)R¹⁴, —N(R¹⁴)S(O)₂R¹⁴, —C(O)OR¹⁴, —OC(O)R¹⁴, —S(O)R¹⁴, —S(O)₂R¹⁴, —NO₂, ═O, ═S, ═N(R¹⁴), —CN; and C₁₋₁₀ alkyl optionally substituted with one or more substituents independently selected from halogen, —OR¹⁴, —SR¹⁴, —N(R¹⁴)₂, —C(O)R¹⁴, —C(O)N(R¹⁴)₂, —N(R¹⁴)C(O)R¹⁴, —C(O)OR¹⁴, —OC(O)R¹⁴, —S(O)R¹⁴, —S(O)₂R¹⁴, —NO₂, ═O, ═S, ═N(R¹⁴), and —CN; or
one R¹ on each of two adjacent carbons come together to form an optionally substituted C₃₋₆ carbocycle together with carbons atoms to which they are attached, wherein the C₃₋₆ carbocycle is optionally substituted with one or more substituents independently selected from halogen, —OR¹⁹, —SR¹⁹, —N(R¹⁹)₂, —C(O)R¹⁹, —C(O)N(R¹⁹)₂, —N(R¹⁹)C(O)R¹⁹, —N(R¹⁹)S(O)₂R¹⁹, —C(O)OR¹⁹, —OC(O)R¹⁹, —S(O)R¹⁹, —S(O)₂R¹⁹, —NO₂, ═O, ═S, ═N(R¹⁹), —CN, C₁₋₆ alkyl, and C₁₋₆ haloalkyl;
R² is selected from hydrogen, halogen, —OR¹⁵, —SR¹⁵, —N(R¹⁵)², —C(O)R¹⁵, —C(O)N(R¹⁵)₂, —N(R¹⁵)C(O)R¹⁵, —N(R¹⁵)S(O)₂R¹⁵, —C(O)OR¹⁵, —OC(O)R¹⁵, —S(O)R¹⁵, —S(O)₂R¹⁵, —NO₂, —CN; and C₁₋₆ alkyl optionally substituted with one or more substituents independently selected from halogen, —OR¹⁵, —SR¹⁵, —N(R¹⁵)₂, —C(O)R¹⁵, —C(O)N(R¹⁵)₂, —N(R¹⁵)C(O)R¹⁵, —C(O)OR¹⁵, —OC(O)R¹⁵, —S(O)R¹⁵, —S(O)₂R¹⁵, —NO₂, ═O, ═S, ═N(R¹⁵), and —CN;
each R³ is independently selected at each occurrence from hydrogen, halogen, —OR¹⁶, —SR¹⁶, —N(R¹⁶)₂, —C(O)R¹⁶, —C(O)N(R¹⁶)₂, —N(R¹⁶)C(O)R¹⁶, —N(R¹⁶)S(O)₂R¹⁶, —C(O)OR¹⁶, —OC(O)R¹⁶, —S(O)R¹⁶, —S(O)₂R¹⁶, —NO₂, —CN; and C₁₋₆ alkyl optionally substituted with one or more substituents independently selected from halogen, —OR¹⁶, —SR¹⁶, —N(R¹⁶)₂, —C(O)R¹⁶, —C(O)N(R¹⁶)₂, —N(R¹⁶)C(O)R¹⁶, —C(O)OR¹⁶, —OC(O)R¹⁶, —S(O)R¹⁶, —S(O)₂R¹⁶, —NO₂, ═O, ═S, ═N(R¹⁶), and —CN; or
R² and one R³ may come together to form C₃₋₆ carbocycle optionally substituted with one or more substituents independently selected from halogen, —OR²⁰, —SR²⁰, —N(R²⁰)₂, —C(O)R²⁰, —C(O)N(R²⁰)₂, —N(R²⁰)C(O)R²⁰, —N(R²⁰)S(O)₂R²⁰, —C(O)OR²⁰, —OC(O)R²⁰, —S(O)R²⁰, —S(O)₂R²⁰, —NO₂, ═O, ═S, ═N(R¹⁹), —CN, C₁₋₆ alkyl, and C₁₋₆ haloalkyl;
each R⁴ is independently selected at each occurrence from halogen, —OR¹⁷, —SR¹⁷, —N(R¹⁷)₂, —C(O)R¹⁷, —C(O)N(R¹⁷)₂, —N(R¹⁷)C(O)R¹⁷, —N(R¹⁷)S(O)₂R¹⁷, —C(O)OR¹⁷, —OC(O)R¹⁷, —S(O)R¹⁷, —S(O)₂R¹⁷, —NO₂, —CN; and C₁₋₁₀ alkyl optionally substituted with one or more substituents independently selected from halogen, —OR¹⁷, —SR¹⁷, —N(R¹⁷)₂, —C(O)R¹⁷, —C(O)N(R¹⁷)₂, —N(R¹⁷)C(O)R¹⁷, —C(O)OR¹⁷, —OC(O)R¹⁷, —S(O)R¹⁷, —S(O)₂R¹⁷, —NO₂, ═O, ═S, ═N(R¹⁷), and —CN;
X¹ is independently selected from N and C(R¹⁰);
X³ is C;
each X² and X⁴ is independently selected from N, N(H) and C(R¹⁰), wherein
when X² is N(H), X⁴ is selected from N and C(R¹⁰); the bond between X² and X³ is a single bond and the bond between X³ and X⁴ is a double bond; or
when X⁴ is N(H), X² is selected from N and C(R¹⁰); the bond between X² and X³ is a double bond and the bond between X³ and X⁴ is a single bond;
wherein at least one of X² and X⁴ is N(H);
R¹⁰ is selected from hydrogen, halogen, —OR¹⁸, —SR¹⁸, —N(R¹⁸)₂, —C(O)R¹⁸, —C(O)OR¹⁸, —NO₂, —CN, C₁₋₆ alkyl, C₁₋₆ haloalkyl, —O—C₁₋₆ haloalkyl, and —O—C₁₋₆ alkyl;
R¹¹, R¹², R¹³, R¹⁴, R¹⁵, R¹⁶, R¹⁷, R¹⁸, R¹⁹, and R²⁰ are each independently selected at each occurrence from:
hydrogen; C₁₋₆ alkyl optionally substituted with one more substituents independently selected from halogen, —O—C₁₋₆ alkyl, —O—C₁₋₆ haloalkyl, —NH₂, —NO₂, ═O, —CN, C₃₋₁₀ carbocycle and 3- to 10-membered heterocycle, wherein each C₃₋₁₀ carbocycle and 3- to 10-membered heterocycle are optionally substituted with one or more substituents independently selected from: halogen, —OH, —O—C₁₋₆ alkyl, —O—C₁₋₆ haloalkyl, —NH₂, —NO₂, ═O, and —CN; and
C₃₋₁₀ carbocycle and 3- to 10-membered heterocycle optionally substituted with one or more substituents independently selected from: halogen, —OH, —O—C₁₋₆ alkyl, —O—C₁₋₆ haloalkyl, —NH₂, —NO₂, ═O, and —CN;
n is selected from 1 and 2;
m is selected from 0, 1 and 2; and
p is selected from 0, 1, 2, 3, 4, 5, and 6.

2. The compound or salt of claim 1, represented by Formula (I-a) or Formula (I-b):

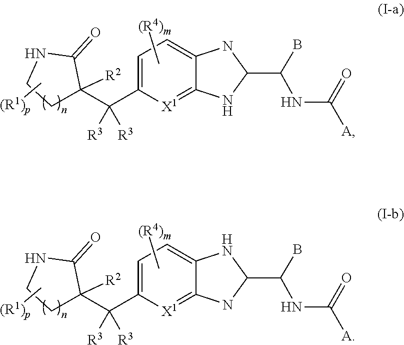

3. The compound or salt of claim 2, wherein X¹ is C(R¹⁰).

4. The compound or salt of or claim 2, wherein X¹ is N.

5. The compound or salt of claim 2, wherein n is 1.

6. The compound or salt of claim 2, wherein n is 2.

7. The compound or salt of claim 2, wherein A is selected from a saturated C₃₋₆ carbocycle and a 5-membered heteroaryl, any of which is optionally substituted with one or more substituents independently selected from:
halogen, —OR¹¹, —SR¹¹, —N(R¹¹)₂, —C(O)R¹¹, —C(O)N(R¹¹)₂, —C(O)OR¹¹, —OC(O)R¹¹, —NO₂, —CN; and C₁₋₆ alkyl optionally substituted with one or more substituents independently selected from halogen, —OR¹¹, —SR¹¹, —N(R¹¹)₂, —C(O)R¹¹, —C(O)N(R¹¹)₂, —C(O)OR¹¹, —OC(O)R¹¹, —NO₂, —CN, C₃₋₁₀ carbocycle and 3- to 10-membered heterocycle; wherein the C₃₋₁₀ carbocycle and 3- to 10-membered heterocycle are each optionally substituted with one or more substituents selected from: —OR¹¹, —SR¹¹, —N(R¹¹)₂, —C(O)R¹¹, —C(O)N(R¹¹)₂, —C(O)OR¹¹, —OC(O)R¹¹, —NO₂, and —CN.

8. The compound or salt of claim 7, wherein A is selected from a saturated C₃₋₆ carbocycle, any one of which is optionally substituted with one or more substituents independently selected from halogen, —OR¹¹, —N(R¹¹)₂, —CN, C₁₋₃ alkyl, and C₁₋₃ haloalkyl.

9. The compound or salt of claim 8, wherein A is selected from cyclopropyl, cyclobutyl, cyclopentyl, and cyclohexyl, any one of which is optionally substituted with one or more substituents independently selected from halogen, —OR¹¹, —N(R¹¹)₂, —CN, C₁₋₃ alkyl, and C₁₋₃ haloalkyl.

10. The compound or salt of claim 9, wherein A is selected from cyclopropyl optionally substituted with one or more substituents independently selected from halogen, C₁₋₃ alkyl, and C₁₋₃ haloalkyl.

11. The compound or salt of claim 10, wherein A is

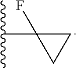 [SMILES: *C1(F)CC1]

12. The compound or salt of claim 7, wherein A selected from pyrazolyl, imidazolyl, oxazolyl, isoxazolyl, oxadiazolyl, triazolyl, and tetrazolyl, any one of which is optionally substituted with one or more substituents independently selected from: C₁₋₆ alkyl optionally substituted with one or more substituents independently selected from halogen, —OR¹¹, —SR¹¹, —N(R¹¹)₂, —C(O)R¹¹, —C(O)N(R¹¹)₂, —C(O)OR¹¹, —OC(O)R¹¹, —NO₂, and —CN.

13. The compound or salt of claim 12, wherein is A selected from pyrazolyl and oxadiazolyl, each of which is optionally substituted with C₁₋₆ alkyl optionally substituted with one or more substituents independently selected from halogen, —OR¹¹, —N(R¹¹)₂, —C(O)R¹¹, —NO₂, and —CN.

14. The compound or salt of claim 13, wherein A is selected from pyrazolyl and oxadiazolyl, each of which is optionally substituted with methyl, ethyl, isopropyl, and propyl.

15. The compound or salt of claim 14, wherein A is selected from:

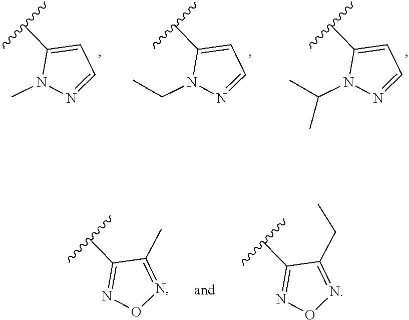

16. The compound or salt of claim 2, wherein B is —C(H)(R⁵)₂.

17. The compound or salt of claim 16, wherein each R⁵ of —C(H)(R⁵)₂ is independently selected from:
halogen, —OR¹³, —N(R¹³)₂, —C(O)R¹³, —C(O)OR¹³, —NO₂, —CN;
C₁₋₆ alkyl optionally substituted with one or more substituents independently selected from halogen, —OR¹³, —N(R¹³)₂, —C(O)R¹³, —C(O)OR¹³, —NO₂, ═O, ═S, ═N(R¹³), and —CN; and
C₃₋₁₀ carbocycle optionally substituted with one or more substituents independently selected from halogen, —OR¹³, —N(R¹³)₂, —C(O)R¹³, —C(O)OR¹³, —NO₂, ═O, ═S, ═N(R¹³), —CN, C₁₋₆ alkyl, and C₁₋₆ haloalkyl.

18. The compound or salt of claim 17, wherein each R⁵ of —C(H)(R⁵)₂ is independently selected from C₃₋₁₀ carbocycle optionally substituted with one or more substituents independently selected from halogen, —OR¹³, —N(R¹³)₂, —C(O)R¹³, —C(O)OR¹³, —NO₂, ═O, ═S, ═N(R¹³), —CN, C₁₋₆ alkyl, and C₁₋₆ haloalkyl.

19. The compound or salt of claim 18, wherein —CH(R⁵)₂ is selected from:

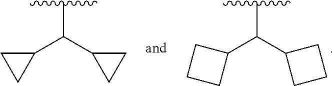 [SMILES: *C(C1CC1)C1CC1 ; *C(C1CCC1)C1CCC1]

20. The compound or salt of claim 17, wherein each R⁵ of —C(H)(R⁵)₂ is independently selected from:
halogen, —OR¹³, —N(R¹³)₂, —C(O)R¹³, —C(O)OR¹³, —NO₂, —CN; and
C₃₋₁₀ carbocycle optionally substituted with one or more substituents independently selected from halogen, —OR¹³, —N(R¹³)₂, —C(O)R¹³, —C(O)OR¹³, —NO₂, ═O, ═S, ═N(R¹³), —CN, C₁₋₆ alkyl, and C₁₋₆ haloalkyl.

21. The compound or salt of claim 20, wherein —CH(R⁵)₂ is

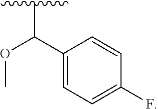 [SMILES: *C(OC)c1ccc(F)cc1]

22. The compound or salt of claim 17, wherein each R⁵ of —C(H)(R⁵)₂ is independently selected from:
C₁₋₆ alkyl optionally substituted with one or more substituents independently selected from halogen, —OR¹³, —N(R¹³)₂, —C(O)R¹³, —C(O)OR¹³, —NO₂, ═O, ═S, ═N(R¹³), and —CN; and
C₃₋₁₀ carbocycle optionally substituted with one or more substituents independently selected from halogen, —OR¹³, —N(R¹³)₂, —C(O)R¹³, —C(O)OR¹³, —NO₂, ═O, ═S, ═N(R¹³), —CN, C₁₋₆ alkyl, and C₁₋₆ haloalkyl.

23. The compound or salt of claim 22, wherein-CH(R⁵)₂ is selected from:

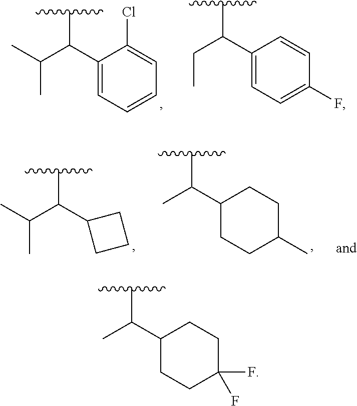

24. The compound or salt of claim 2, wherein B is C₆₋₁₀ carbocycle optionally substituted with one or more substituents independently selected from: halogen, —OR¹², N(R¹²)₂, —C(O)R¹², —NO₂, ═O, ═S, ═N(R¹²), —CN, and C₁₋₆ alkyl optionally substituted with one or more substituents independently selected from halogen, —OR¹², N(R¹²)₂, —C(O)R¹², —NO₂, ═O, ═S, ═N(R¹²), and —CN.

25. The compound or salt of claim 24, wherein B is cyclohexyl, cycloheptyl, dispiro[2.0.2⁴.1³]heptane, spiro[3.3]heptane, and indane, any one of which is optionally substituted with one or more substituents independently selected from: halogen, —OR¹², N(R¹²)₂, —NO₂, —CN, and C₁₋₆ alkyl optionally substituted with one or more substituents independently selected from halogen, —OR¹², N(R¹²)₂, —C(O)R¹², —NO₂, ═O, ═S, ═N(R¹²), and —CN.

26. The compound or salt of claim 25, wherein B is selected from:

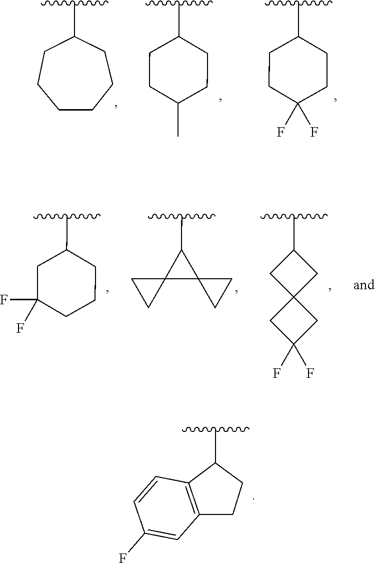

27. The compound or salt of claim 2, wherein m is 1.

28. The compound or salt of any one of claim 2, wherein R⁴ is selected from halogen, —OR¹⁷, —N(R¹⁷)₂, —NO₂, —CN, C₁₋₃ alkyl, and C₁₋₃ haloalkyl.

29. The compound or salt of claim 28, wherein R⁴ is selected from chloro, fluoro, methyl, ethyl, and —CN.

30. The compound or salt of claim 2, wherein m is 0.

31. The compound or salt of claim 2, wherein each R³ is independently selected at each occurrence from hydrogen, halogen, —OR¹⁶, —N(R¹⁶)₂, —NO₂, —CN, C₁₋₃ alkyl, C₁₋₃ alkyl-OR¹⁶, and C₁₋₃ haloalkyl.

32. The compound or salt of claim 31, wherein each R³ is independently selected at each occurrence from hydrogen and methyl.

33. The compound or salt of claim 2, wherein R² is selected from hydrogen and methyl.

34. The compound or salt of claim 2, wherein p is selected from 0, 1, 2, 3, and 4.

35. The compound or salt of claim 2, wherein R¹ is selected from halogen, —OR¹⁴, —N(R¹⁴)₂, —NO₂, —CN, C₁₋₆ alkyl, and C₁₋₆ haloalkyl.

36. The compound or salt of claim 35, wherein R¹ is selected from hydrogen and —CF₃.

37. The compound or salt of claim 2, wherein one R¹ on each of two adjacent carbons come together to form an optionally substituted C₃₋₆ carbocycle together with carbons atoms to which they are attached, wherein the C₃₋₆ carbocycle is optionally substituted with one or more substituents independently selected from halogen, —OR¹⁹, —N(R¹⁹)₂, —NO₂, —CN, C₁₋₆ alkyl, and C₁₋₆ haloalkyl.

38. The compound or salt of claim 1 selected from the group consisting of: 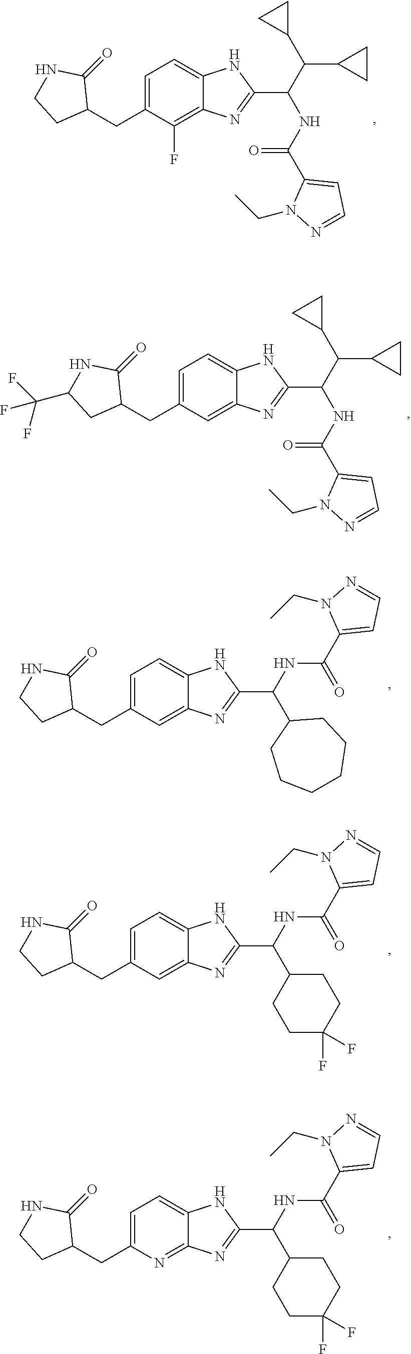 [SMILES: CCn1nccc1C(=O)NC(c1nc2c(F)c(CC3CCNC3=O)ccc2[nH]1)C(C1CC1)C1CC1 ; CCn1nccc1C(=O)NC(c1nc2cc(CC3CC(C(F)(F)F)NC3=O)ccc2[nH]1)C(C1CC1)C1CC1 ; CCn1nccc1C(=O)NC(c1nc2cc(CC3CCNC3=O)ccc2[nH]1)C1CCCCCC1 ; CCn1nccc1C(=O)NC(c1nc2cc(CC3CCNC3=O)ccc2[nH]1)C1CCC(F)(F)CC1 ; CCn1nccc1C(=O)NC(c1nc2nc(CC3CCNC3=O)ccc2[nH]1)C1CCC(F)(F)CC1] 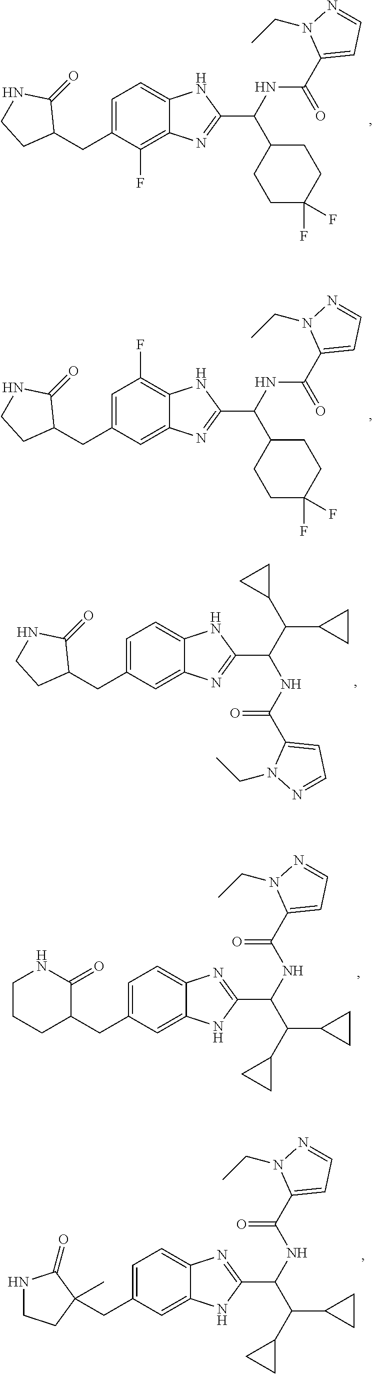 [SMILES: CCn1nccc1C(=O)NC(c1nc2c(F)c(CC3CCNC3=O)ccc2[nH]1)C1CCC(F)(F)CC1 ; CCn1nccc1C(=O)NC(c1nc2cc(CC3CCNC3=O)cc(F)c2[nH]1)C1CCC(F)(F)CC1 ; CCn1nccc1C(=O)NC(c1nc2cc(CC3CCNC3=O)ccc2[nH]1)C(C1CC1)C1CC1 ; CCn1nccc1C(=O)NC(c1nc2ccc(CC3CCCNC3=O)cc2[nH]1)C(C1CC1)C1CC1 ; CCn1nccc1C(=O)NC(c1nc2ccc(CC3(C)CCNC3=O)cc2[nH]1)C(C1CC1)C1CC1] 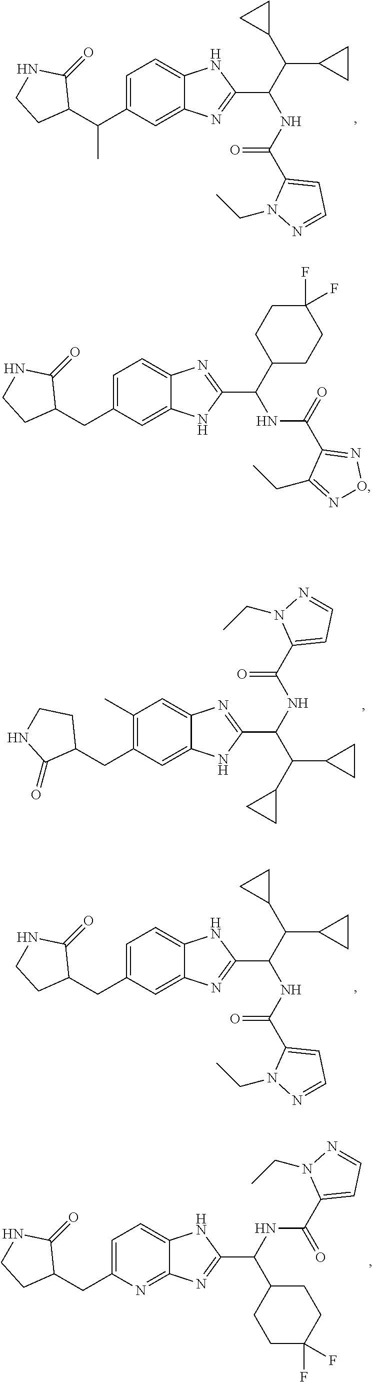 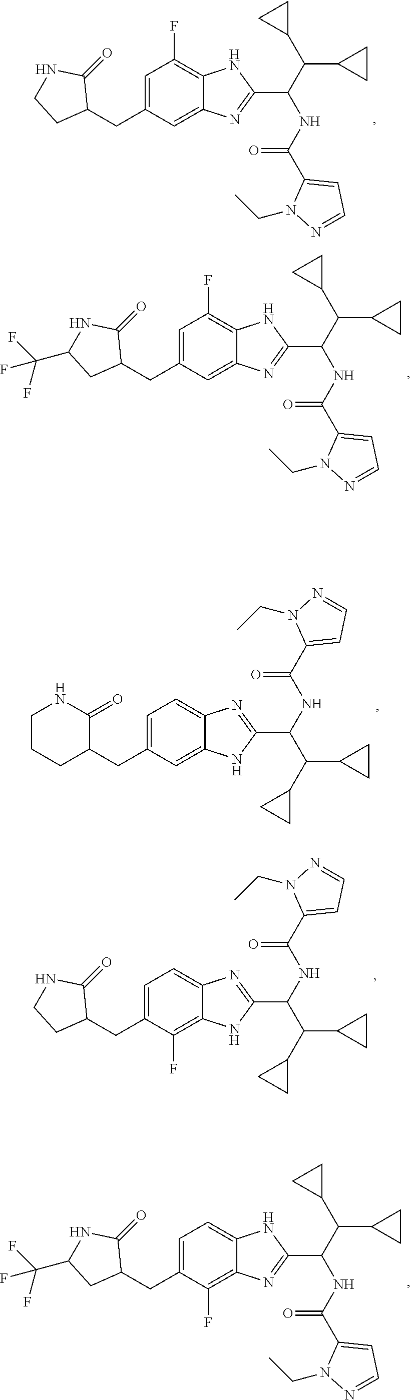 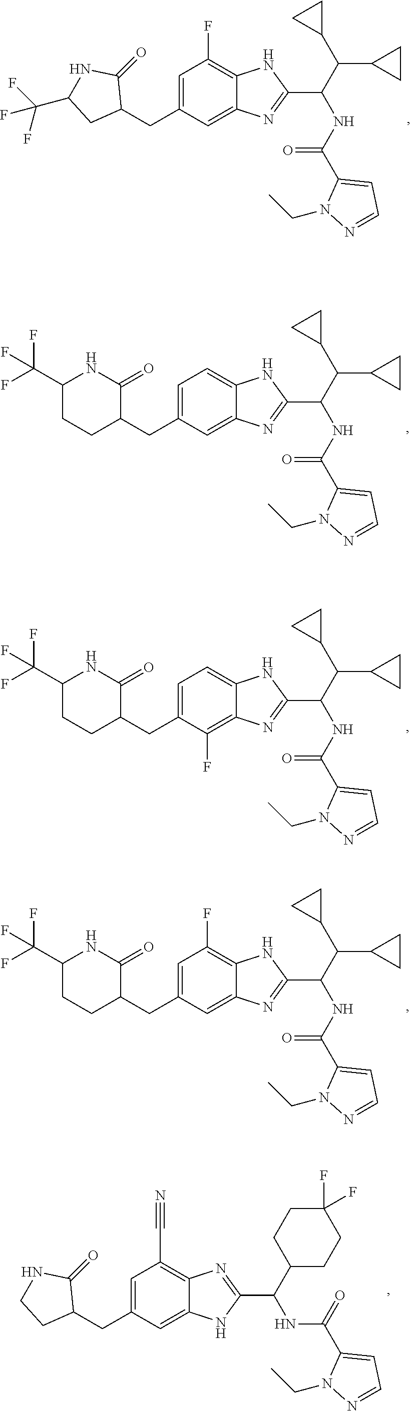 [SMILES: CCn1nccc1C(=O)NC(c1nc2cc(CC3CC(C(F)(F)F)NC3=O)cc(F)c2[nH]1)C(C1CC1)C1CC1 ; CCn1nccc1C(=O)NC(c1nc2cc(CC3CCC(C(F)(F)F)NC3=O)ccc2[nH]1)C(C1CC1)C1CC1 ; CCn1nccc1C(=O)NC(c1nc2c(F)c(CC3CCC(C(F)(F)F)NC3=O)ccc2[nH]1)C(C1CC1)C1CC1 ; CCn1nccc1C(=O)NC(c1nc2cc(CC3CCC(C(F)(F)F)NC3=O)cc(F)c2[nH]1)C(C1CC1)C1CC1 ; CCn1nccc1C(=O)NC(c1nc2c(C#N)cc(CC3CCNC3=O)cc2[nH]1)C1CCC(F)(F)CC1] 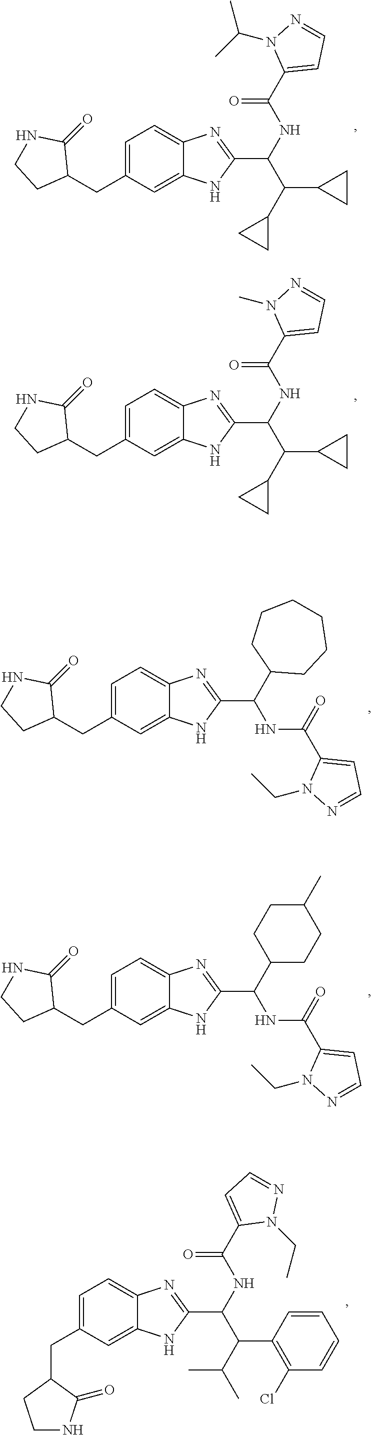 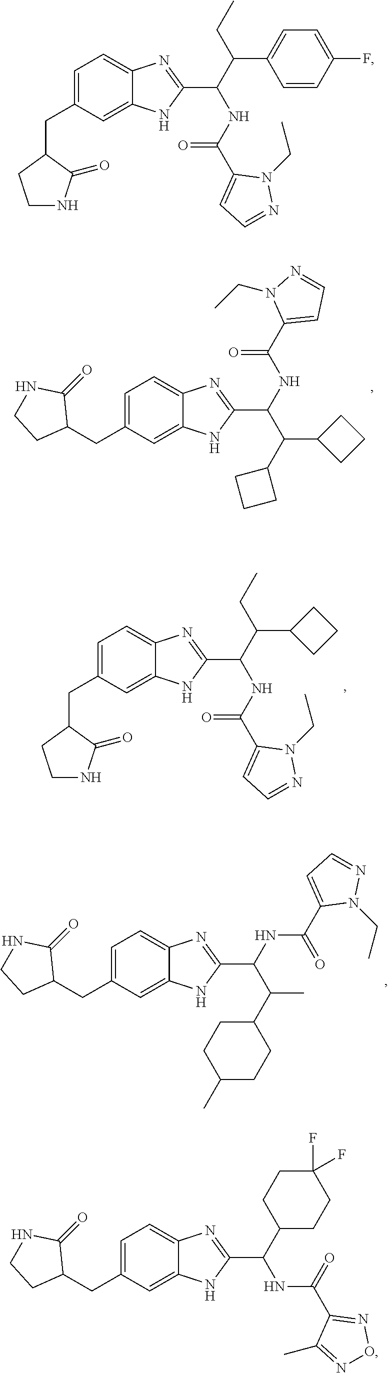 [SMILES: CCC(c1ccc(F)cc1)C(NC(=O)c1ccnn1CC)c1nc2ccc(CC3CCNC3=O)cc2[nH]1 ; CCn1nccc1C(=O)NC(c1nc2ccc(CC3CCNC3=O)cc2[nH]1)C(C1CCC1)C1CCC1 ; CCC(C1CCC1)C(NC(=O)c1ccnn1CC)c1nc2ccc(CC3CCNC3=O)cc2[nH]1 ; CCn1nccc1C(=O)NC(c1nc2ccc(CC3CCNC3=O)cc2[nH]1)C(C)C1CCC(C)CC1 ; Cc1nonc1C(=O)NC(c1nc2ccc(CC3CCNC3=O)cc2[nH]1)C1CCC(F)(F)CC1] 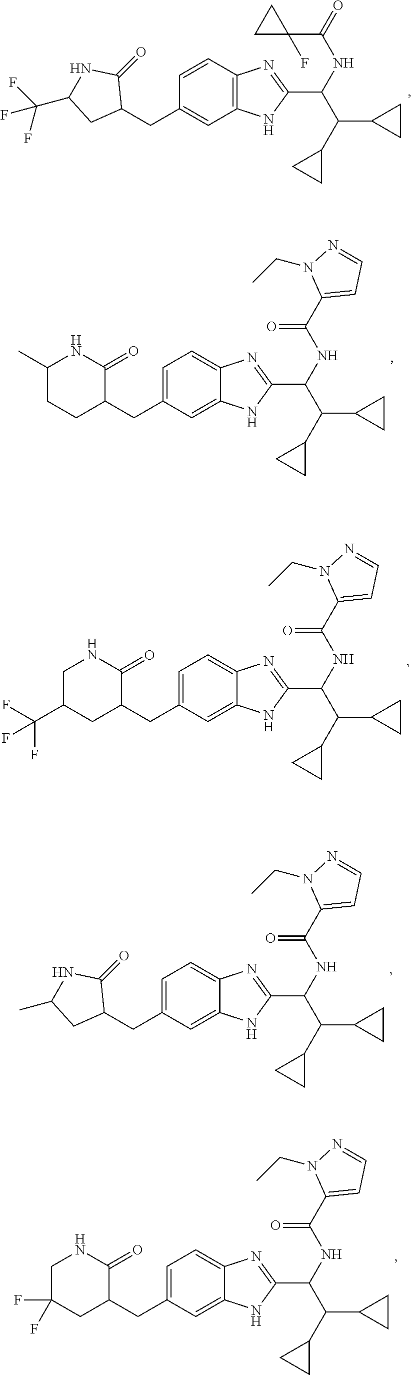 [SMILES: O=C1NC(C(F)(F)F)CC1Cc1ccc2nc(C(NC(=O)C3(F)CC3)C(C3CC3)C3CC3)[nH]c2c1 ; CCn1nccc1C(=O)NC(c1nc2ccc(CC3CCC(C)NC3=O)cc2[nH]1)C(C1CC1)C1CC1 ; CCn1nccc1C(=O)NC(c1nc2ccc(CC3CC(C(F)(F)F)CNC3=O)cc2[nH]1)C(C1CC1)C1CC1 ; CCn1nccc1C(=O)NC(c1nc2ccc(CC3CC(C)NC3=O)cc2[nH]1)C(C1CC1)C1CC1 ; CCn1nccc1C(=O)NC(c1nc2ccc(CC3CC(F)(F)CNC3=O)cc2[nH]1)C(C1CC1)C1CC1] 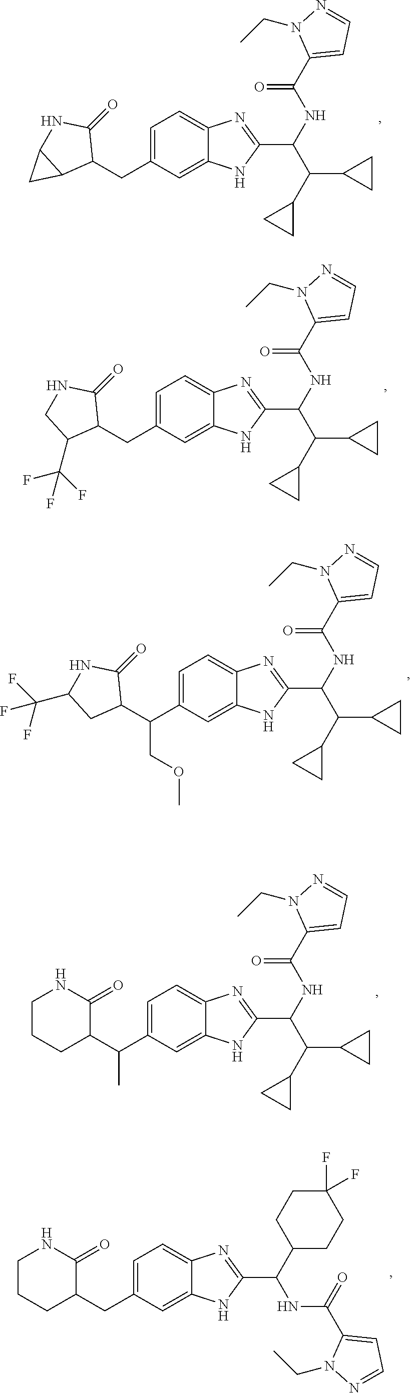 [SMILES: CCn1nccc1C(=O)NC(c1nc2ccc(CC3C(=O)NC4CC43)cc2[nH]1)C(C1CC1)C1CC1 ; CCn1nccc1C(=O)NC(c1nc2ccc(CC3C(=O)NCC3C(F)(F)F)cc2[nH]1)C(C1CC1)C1CC1 ; CCn1nccc1C(=O)NC(c1nc2ccc(C(COC)C3CC(C(F)(F)F)NC3=O)cc2[nH]1)C(C1CC1)C1CC1 ; CCn1nccc1C(=O)NC(c1nc2ccc(C(C)C3CCCNC3=O)cc2[nH]1)C(C1CC1)C1CC1 ; CCn1nccc1C(=O)NC(c1nc2ccc(CC3CCCNC3=O)cc2[nH]1)C1CCC(F)(F)CC1] 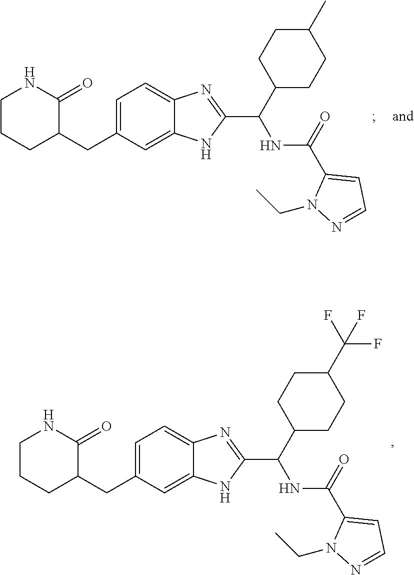 [SMILES: CCn1nccc1C(=O)NC(c1nc2ccc(CC3CCCNC3=O)cc2[nH]1)C1CCC(C)CC1 ; CCn1nccc1C(=O)NC(c1nc2ccc(CC3CCCNC3=O)cc2[nH]1)C1CCC(C(F)(F)F)CC1]
or pharmaceutically acceptable salts thereof.

39. A pharmaceutical composition comprising a compound or salt of claim 2 and a pharmaceutically acceptable excipient.

40. A method of modulating IL-17A in a subject in need thereof, comprising administering to the subject a compound or salt of claim 2.

41. A method of treating an inflammatory disease or condition comprising administering to a subject in need thereof a compound or salt of claim 2.

42. The method of claim 41, wherein the inflammatory disease or condition is selected from plaque psoriasis, guttate psoriasis, inverse psoriasis, pustular psoriasis, erythrodermic psoriasis, psoriatic arthritis, ankylosing spondylitis, hidradenitis suppurativa, rheumatoid arthritis, palmoplantar psoriasis, spondyloarthritis, and Non-infectious Uveitis.
